SEQUENCE LISTING (113 sequences)

SEQ ID NO: 1 - protein, 479 aa, Pepper cryptic Virus 1
MVRGTLVGYDYTQFQGDLVKSTHRHPHVVHREIATTYVDQYAYEHIETFSSLYPELILKGWSRSYYLPEKHLAAVLNYSMPNVPASQLSQSLYRQAIESAKNGFISLPRVKAFDVLTEMDQVPFKSSSSAGYNYTGRKGLIGDENHSRAISIAKAVLWSAIKDDGEGIEHVIRTSVPDVGYTRTQLTDLLEKTKVRQVWGRAFHYILLEGLVAYPFIQTVMSHKTFIHAGQDPLISVPRLLSDVALNCKWIYSLDWSQFDATVSRFEIHAAFDIIKSYVDFPNYETEQAFEITRQLFIHKKVAVPDGYIYESHKGIPSGSYYTSLVGSIINYLRINYLWRLLTGHPPQQCHTLGDDSLVGDNSYVNPQAIEEAANKLGWHFNPDKTQYSTVPEEITFLGRTYVGGLNKRDLTKCIRLLVYPEYPVESGRISAYRAKSIAQDAGGLSEVLNRIADKLRRIYGTASEEEVPIYFKRYVFGV

SEQ ID NO: 2 - protein, 539 aa, Pseudogymnoascus destructans partitivirus-pa
MEVSPFDPTPLDNVIEGSPLVDDSLLVPSSRTRGSSYDVIPEHFNSPGLTEIARYGGYPVYSGGSNTDAWVRTSLKEFDRTMYENIYGYTRKPEGPQGMYKSLLKFSEDKSTFHSLNRVQRRAMIGAIKKARTAFKLPWKREPLDWHEVGQFLRRDTAAGATFMGKKKGDVMEEIYHEARWLGHRMKQDGREKFNPKKMRFPPCLAGQRGHMSERDTPKTRLVWVYPAEMLCVEGFYAPQMYRDFMNDRHTPMLNGKSSQRLYTEWCVGLREGEKLYGLDFSSFDSKVPSWLIRVAFDILRQNIEWSTFRGEKVSKREAQKWRNVWDAMVYYFINTPMLMPDGRMFRKRRGVPSGSWWTQMIDSVVNYILVDYLTQCQTCQIRGLRVLGDDSAFRSCHDFSLDQASADAAAVLMILNPDKCEVTLDPTKFKLLGTTYEDGHPHRETIDWFKFALYPESSVSSIDVSLTRLVGLWLGGGMWDLHFCKFMDYFQTCFPCPLEGWFSKDQRRWLEVIFSGKAPRGWTTKKSLFWHSIFYTYC

SEQ ID NO: 3 - protein, 610 aa, artificial sequence
  RdRp with RT consensus sequence
  misc_feature (7)..(7): Xaa can be any naturally occurring amino acid
  misc_feature (16)..(16): Xaa can be any naturally occurring amino acid
  misc_feature (24)..(24): Xaa can be any naturally occurring amino acid
  misc_feature (53)..(53): Xaa can be any naturally occurring amino acid
  misc_feature (76)..(76): Xaa can be any naturally occurring amino acid
  misc_feature (101)..(101): Xaa can be any naturally occurring amino acid
  misc_feature (151)..(151): Xaa can be any naturally occurring amino acid
  misc_feature (169)..(169): Xaa can be any naturally occurring amino acid
  misc_feature (171)..(171): Xaa can be any naturally occurring amino acid
  misc_feature (185)..(185): Xaa can be any naturally occurring amino acid
  misc_feature (310)..(310): Xaa can be any naturally occurring amino acid
  misc_feature (356)..(356): Xaa can be any naturally occurring amino acid
  misc_feature (383)..(383): Xaa can be any naturally occurring amino acid
  misc_feature (413)..(413): Xaa can be any naturally occurring amino acid
  misc_feature (423)..(423): Xaa can be any naturally occurring amino acid
  misc_feature (455)..(455): Xaa can be any naturally occurring amino acid
  misc_feature (457)..(457): Xaa can be any naturally occurring amino acid
  misc_feature (465)..(465): Xaa can be any naturally occurring amino acid
  misc_feature (513)..(513): Xaa can be any naturally occurring amino acid
  misc_feature (524)..(524): Xaa can be any naturally occurring amino acid
  misc_feature (575)..(575): Xaa can be any naturally occurring amino acid
  misc_feature (590)..(590): Xaa can be any naturally occurring amino acid
  misc_feature (595)..(595): Xaa can be any naturally occurring amino acid
  misc_feature (609)..(609): Xaa can be any naturally occurring amino acid
MSMPFNXVRNYLAERXIRLKREWXTYQSSDRDPDATLMESFDQDPTRHYVNAXESHLIDALNMEYLTLVFARRTSXDRKPTNFEFGIPPGLIPINGIASGXHTDPWIRELLKYLYPEYEQILNGYTRSPGTTDAIFKDFFKFDIPSHPIPXDRHYRAILKAKKAFRPPXPXRPLHFLDVRFYKWXLSAGAPFSGRPKKGYLYNEIFSEARTLVHRIKQGFGFDPGLERSPPTLLHTRTHISKRDDPKVRPVWGVPKEFIIAEGMLAWPLIAQFRKPSTPMLWGYETIRGGWRRLNNLAEGSTFYTLDWSXFDQRVPFWLIRDAFDILRSLIDFNNGYGPTVEYPDAQRLENLWDFXVEWFINTPILLPDGRMYRKFAGIPSGXFNTQLLDSFVNYILIIYLLSEFGFDPREIXVQGDDSLIRXFTLIPLDRFLEKYALERFGMVLNPEKSEITNXPXGIEVLGYXNNNGLPHRDEIKLLAQLLYPERVPTPEISMARAIGIAYASCGMDDRVXCEDIYNYLRSXYPPPEAGLFSKFQDRWDEIVPDHFPTGWEVRRYLYGLFWPSIHFINGPDEXPPSSKTMRDYEIENXLPSRXAPTFETVVPSTRNXP

SEQ ID NO: 4 - protein, 9 aa, artificial sequence
  Ty 1 element consensus RT motif from yeast
  misc_feature (1)..(1): Xaa can be any naturally occurring amino acid
XADTFRDLR

SEQ ID NO: 5 - protein, 15 aa, artificial sequence
  Ty element from yeast with RdRp RT motif
FVNVYLDDILIFSES

SEQ ID NO: 6 - protein, 12 aa, artificial sequence
  Ty  element with RdRp Rt domain
KHLDTVLERLKN

SEQ ID NO: 7 - protein, 13 aa, artificial sequence
  Ty element from yeast, RdRp RT conserved domain
  misc_feature (9)..(9): Xaa can be any naturally occurring amino acid
ENLIVKKKXCKFA

SEQ ID NO: 8 - protein, 7 aa, artificial sequence
  Ty element with RdRp RT domain
EETEFLG

SEQ ID NO: 9 - protein, 9 aa, Pepper cryptic virus
INYLWRLLT

SEQ ID NO: 10 - protein, 15 aa, Pepper cryptic virus
QCHTLGDDSLVGDNS

SEQ ID NO: 11 - protein, 12 aa, Pepper cryptic virus
VNPQAIEEAANK

SEQ ID NO: 12 - protein, 13 aa, Pepper cryptic virus
LGWHFNPDKTQYS

SEQ ID NO: 13 - protein, 7 aa, Pepper cryptic virus
EEITFLG

SEQ ID NO: 14 - protein, 9 aa, Caprine arthritis encephalitis lentivirus
MQEILEDWI

SEQ ID NO: 15 - protein, 15 aa, Caprine arthritis encephalitis lentivirus
QFGIYMDDIYIGSDL

SEQ ID NO: 16 - protein, 12 aa, Caprine arthritis encephalitis lentivirus
EIVKDLANYIAQ

SEQ ID NO: 17 - protein, 13 aa, Caprine arthritis encephalitis lentivirus
YGFTLPEEKRQKG

SEQ ID NO: 18 - protein, 7 aa, Caprine arthritis encephalitis lentivirus
YPAKWLG

SEQ ID NO: 19 - protein, 9 aa, Bovine leukemia virus
LQEPLRQVS

SEQ ID NO: 20 - protein, 15 aa, Bovine leukemia virus
LLVSYMDDILYVSPT

SEQ ID NO: 21 - protein, 12 aa, Bovine leukemia virus
QCYQTMAAHLRD

SEQ ID NO: 22 - protein, 13 aa, Bovine leukemia virus
LGFQVASEKTRQT

SEQ ID NO: 23 - protein, 7 aa, Bovine leukemia virus
SPVPFLG

SEQ ID NO: 24 - protein, 9 aa, Saccharomyces cerevisiae
INTILGEAK

SEQ ID NO: 25 - protein, 15 aa, Saccharomyces cerevisiae
HVVCYMDDILIHSKS

SEQ ID NO: 26 - protein, 12 aa, Saccharomyces cerevisiae
KHVKDVLQKLKN

SEQ ID NO: 27 - protein, 13 aa, Saccharomyces cerevisiae
ANLIINQAKCEFH

SEQ ID NO: 28 - protein, 7 aa, Saccharomyces cerevisiae
SQVKFIG

SEQ ID NO: 29 - protein, 9 aa, Rice tungro bacciliform virus
MQESFGDLK

SEQ ID NO: 30 - protein, 15 aa, Rice tungro bacciliform virus
FALLYIDDILIASNN

SEQ ID NO: 31 - protein, 12 aa, Rice tungro bacciliform virus
EHLKIFFNRVKE

SEQ ID NO: 32 - protein, 13 aa, Rice tungro bacciliform virus
VGCVLSKKKSKMF

SEQ ID NO: 33 - protein, 7 aa, Rice tungro bacciliform virus
KEVEYLG

SEQ ID NO: 34 - protein, 9 aa, E. coli
LDGLEALLA

SEQ ID NO: 35 - protein, 15 aa, E. coli
NYVRYADDFIITGES

SEQ ID NO: 36 - protein, 12 aa, E. coli
QVLPVVRRFMAE

SEQ ID NO: 37 - protein, 13 aa, E. coli
RGLMLSPEKTKIT

SEQ ID NO: 38 - protein, 7 aa, E. coli
EGFDFLG

SEQ ID NO: 39 - protein, 665 aa, Atkinsonella hypxylon virus
MSTLLIPQDTIAHTFDEAVASESNLRIDEVPENYLERFIHPSEPENFEFYSLRDSDIPSKRIPKNGIQVFENLKYHTNSKDNLYKDQPSSGPSPMRGVANIIREYFPQYLDDLRTWCRPKSSDDSIFNDFNHEQRITQPFTEERERRLLPLIDHFLGIKPYDIVHYCDTRFYPWKLSTRADYFHNHSRDRKAHAAKSHPDFATGPTKKSYFINSHLFFDRSTVHNIKEYGFPFRPTTDSARNETLLDLWFKKVPTELLVRSHISKRDNLKVRPVYNAPMIYIRIECMLFYPLLAQARKRDCCIMYGLETIRGGMNELERISNAFNSFLLIDWSRFDHLAPFTISNFFFKKWLPTKILIDHGYAQISNYHDHVHSFSAQAQSHGIPMISKEYQTPPEATVFAKKVLNLISFLERWYRDMVFVTPDGFAYRRTHAGVPSGILMTQFIDSFVNLTILLDGLIEFGFTDEEIKQLLVFIMGDDNVIFTPWTLLKLIEFFDWFAKYTLDRFGMVINISKSAVTSIRRKIEVLGYTNNYGFPTRSISKLVGQLAYPERHVTDADMCMRAIGFAYASCAQSETFHALCKKVFQYYFAKTSINERLILKGRKAELPGMFFAYPDVSEHIRLDHFPSLSEVRILLSKFQGYLKETPFGTIPTFSTPQTLRDQTQ

SEQ ID NO: 40 - protein, 745 aa, Cannabis cryptic virus Fedora
MPYNAVRNYLAERMIRTKREWQLYQSSNRDPESTLEHYQDSDYLRYYQNARFNLNQEERFRALNKEYSTLVEAFRTDNLRKHQPYELHQPIPPDAAPIPDKRQPAPGIKLVPLMYHYGHVVHDTPSTTDDHTSNDDSQSAPSRLLKTTEFGYSIDKQIYDLVCRRYPTYLSVINLYCRPLGTVDATFSDFNKEQIPSNPIDPDRKEHVLKHIFKFLDATPYLPVHFVDTQFCKTPLVTGTGYHNRYSFKQKAHAKYSHPEEYATYPSSKGYFYNATYENARTLVHYIKQNGLPFEFDFNLSEESLTDEQIDLFVNRSNAFFNDYPTLLFTRNHISKRTGPLKVRPVYAVDDIFIIIELMLTFPLTGQARKPSCWIRMARNHSGSNRYIDRLGRSYSTFFFINWASYDQPMPRVITDIYYTDFLRSLIVINHGYQPTYEYSFYPDLDEHKLYDRKNNLLFFLHTWYNNMTFLLPDGFAYRRSYCGVPSGLYNTQYLDSFGNLFLIIDAMIEFGFTDSEIDGFVLLILGDDNTGMTQMHIHRISQFINFLEKYALERYNMVLSATKSVLTTLRSKIETLGYQCNYGSPKRDIDKLVAQLCFPENGLKPHTMSARAVGIAYAAAGQDYVFHSFCQDVYNMFRSYYKPDARANLFFQRQVLQNLEDGIPDLATPVVPPFPSLFEVREMYSEYKGPLSFEPKWNKAHFINDPNDIPPFSKTMRDYEKEHNLPVRVSPTFETVVPSTKNLP

SEQ ID NO: 41 - protein, 746 aa, Cannabis cryptic virus Hemp
MPYNAVRNYLAERMIRTKREWQLYQSSNRDPESTLEHYQDSDYLRYYQNARFNLNQEERFRALNKEYSTLVEAFRTDNLRKHQPYELHQPIPPDAAPIPDKRQPAPGIKLVPLMYHYGHVVHDTPSTTDDHTSNDDSQSAPSRLLKTTEFGYSIDKQIYDLVCRRYPTYLSVINLYCRPLGTVDATFSDFNKEQIPSNPIDPDRKEHVLKHIFKFLDATPYLPVHFVDTQFCKTPLVTGTGYHNRYSFKQKAHAKYSHPEEYATYPSSKGYFYNATYENARTLVHYIKQNGLPFEFDFNLSEESLTDEQIDLFVNRSNAFFNDYPTLLFTRNHISKRTGPLKVRPVYAVDDIFIIIELMLTFPLTVQARKPSCCIMYGFETIRGSNRYIDRLARSYSTFFSLDWSSYDQRLPRVITDIYYTDFLRSLIVINHGYQPTYEYSSYPDLDEHKLYDRMNNLLFFLHTWYNNMTFLLPDGFAYRRSYCGVPSGLYNTQYLDSFGNLFLIIDAMIEFGFTDSEIDGFVLLILGDDNTGMTQMHIHRISQFINFLEKYALERYNMVLSATKSVLTTLRSKIETLGYQCNYGSPKRDIDKLVAQLCFPENGLKPHTMSARAVGIAYAAAGQDYVFHSFCQDVYNMFRSYYKPDARANLFFQRQVLQNLEDGIPDLATPVVPPFPSLFEVREMYSEYKGPLSFEPKWNKAHFINDPNDIPPFSKTMRDYEKEHNLPVRVSPTFETVVPSTKNLP

SEQ ID NO: 42 - protein, 746 aa, Crimson clover cryptic virus 2 RdRp
MPFNAVRNYLNERMTRLKQEWKTYQSTDHDPIKILDTIQDPDYRRYLDNARFNSDNEMKHLMLNKEYSTLVEAYRTDNAKKHQVYELHQPIPNDAAPVIQSRLPAKGIKLVPLMYHYGHVTHDPVSLTDANRSDDFETQVVPDSDAPTRFGYPIDVRIYNLICYRYSKYLEVINAYCRPIGTVNATFEDFNKEQIPSAPIDPKRKENVLSHIHKFLDTKPYLPLHFVDTQFCKTPLVTGTGYHNRYSFKQKAHAKYSHPAEYATKPISKGYFYNATYENARTIIHFIKEYGLPFNVIRADDKSELTDSDVQKYINEANSFFNDYPTLLFTRNHISKRDGPLKVRPVYAVDDIFIIMELMLTFPLTIQARKPSCCIMYGLETIRGSNRYIEQIARDYSTFFSLDWSSYDQRLPRVITDIYYTDFLRSLLVINNGYQPTYEYPTYPDLDEHKLYHRMDNLLHFLHLWYNNMTFLLPDGYAYRRTSCGVPSGLYNTQYLDSFGNLFLIIDAMLEFGFTDSEIQKFILLVLGDDNTGMTTIPICRMFNFITFLEKYALERYNMVLSTTKSVLTTFRSKIESLGYQCNHGSPKRDIDKLVAQLCYPENGLKPHTMAARAIGIAYASAGQDDMFHSFCQDVYNIFRSDYRPDDRMNLHFKRQIFHNLEDGMPDLAPPIVPPFPSLYEIREMYAYYKGPLDFAPKWNYAHFMSDPDTTPPYSKTMRDYEAENNISSRVAPTFETVVPSTINLP

SEQ ID NO: 43 - protein, 663 aa, Ceratocystis resinifera partitivirus
MPSFSANPTYQTLIDDIDFDVPIAHPFSIINTDLQPVDDEETVHDLGSKDFEFYKVVSNDLPLNRAPSVGIESLPNIRYHNHSNDHRYRDQPPTGPPPMRGVHRIINDSFPQYLPYLKEWCRPKTSSDAIFEDFNQPQIPSIPLQHSRKQKILKLVNHFMGVKPYDIVHFCDTRFYPWDLSKKADYFHNHSNARKRHAQTSHASTATGPTKKSWFINAHLFHDRSTVHNIKLYGLPFKPHSYAARNKILQELWFKKIPTELLVRSHISNPKKLKVRPVNNAPMIFLRIECMLFYPLLAQLRKQQCSIMYGLETIRGGMMEIESLATRFSNFMMIDWSKFDQTVPFTLVDMFYQDWIPTLILVDSGYAKIHNYNDHVHSFAAQARKLGVHGNSNLNEAPPETAIFANKVENLLKFINTWFKEMVYITPDGFAYRRTFAGVPSGILCTQLIDSFVNLVVLIDSLFEFGFHESDITSALILLMGDDNVVFAPDKLSQLHSFFLFLPGYAKKRWNMTVNVDKSIFTTLRRKIEILGYTNNYGMPVRSLSKLIGQLAYPERHGFDSDMCMRAIGFAWCAAACDSTFHDFCRKVFMYYYARVNVPIKDLVQANASALPGMFFAYRDVHQHIKLDHFPSIEEVRQVLSKHHGYLTEEPLWKYDFFIHPKP

SEQ ID NO: 44 - protein, 673 aa, Fusarium poae virus 1
MLANIRDYFHEKLTRLLYDHKIFQSNSKDPDLTLEAHHSSDIERIYKSIHYDFNRSPAPIDYEAQYQSIKHILEDKQSQQGFPHEYYRLHESPIPDDRIPPSGIKLLPFEYKSMNVVTATPEVPESGFKIHPRIERLLRSKYPQYLQYVRKYTRPLGTTNATVSDFFKPQTPSQPVEPTRINHVMSHVMKKMAITPYLPLHFVDTQYDKRPLANGTGYHNRRSHEMNIHALFSHPKEYESKRTSKGYYVNAFLESARSLIHWIKLYGNPFRHCPSDLAQSLREFFLQRPTMLFTRNHISDRDGILKQRPVYAVDDLFLTIESMLTFPAHVIARKPECCIMYGLETIRGSNQILDKIASDYKSFFTIDWSGFDQRLPWVIVKLFFTEYIPRLLVVNHGYAPTYEYPSYPDLTTNDMVSRLTNLITFLATWYFNMVFVTADGFSYVREHAGVPSGMLNTQFLDSFGNLFLLIDGLIEFGSTDAEIDDILLFIMGDDNSAFTTWSITHLEQFVSFFETYALSRYGMVLSKTKSIITTLRHKIETLSYQCNFGHPRRPIGKLVAQLCFPERGPRPKYMSARAVGMAWASCGQDKTFHDFCRDVYHEFNDDRADLDESAYLHIQSHLPGYLKIDESVRQIVDFQVFPSQQTVYHTVSRWKGPLSYQPEMGSCSLCQPT

SEQ ID NO: 45 - protein, 704 aa, Heterobasidion partitivirus 8
MLSKVRDFFHEKLSRLLLQHSIFQSNDKDEEQTLLENQSSDVKRIYNSIHYSFQTDDTQAAYEDQYQHIKHVLEDKDRLSNFPAEFYLPYDTTTTPDNRVPPSGIDQLPYVYKRTNVVTATDEVPETGYPIQNRLLRLIRSRYPQYLPHVRTFTRPLGTTDATVSDFFKPQHPSRLVDPSRISHVMKHVMNKMAITPYLPIHFVDTQYDKRPLSTGTGYYNRRSHEANIHALYSHPKEYENKRTSKGYYINAFLESARSLVHWIKSYGNPFRSKPADPRESLKKFFLQRPTMLFTRNHISKILGALKQRPVYAVDDLFLTLESMVTFPAHTIARKIECCIMYGYETIRGSNVQLDRLAQRYNSFFTIDWSGFDQRLPWVIVLLFFTEFLPRLLVINHGYAPTYDFPSYPDLTTEMMYQRLSNILSFLATWYFNMVFITADGFAYVRRFAGVPSGLLNTQFLDSFGNLFLIIDALIEFGAQDEEIDSILLLIMGDDNSGFTIWSIARLEQFITFLESYALTRYGMVMSKTKSLVTVLRHKIQTLSYTCNFGRPLRPIPELVAHLVFPEREFKPQFMSARAVGMAWASCAQDKTFHDFCRDVFYEYLEESVPVDNTNIAWIQSHLPGYLRVDPEVTKMIDLNVFPSFLHVSQKLSRWQGPLSYQPKWDLAHFINQPDVIPDDSITMFEYMQEHSLSLDIQFDLFSA

SEQ ID NO: 46 - protein, 746 aa, Hop trefoil virus 2
MPFNSVRNYLEERSVRVKKEMMTYQSSNRDPDAILEQSQDPDYRRYYDNARYNPSNDLKYRMLSKEYSTLVEAYRLRNDDKHQPYELHQPVPQDAAPVPSYRAPAPGIKPVPLMYHYGHVIHDPVSLTESAIDDDSDTLPDSDEPTVTHFGYPINKRIYDLICNRYPEYLSVISAYCRPIGTVDATFKDFNKEQIPSGPIDPNRKEEVLTHIFRFLDAQPYLPLHFVDTQFCKTPLVTGTGYHNRYSFKQKAHAKYSHPEEYAQMPTSKGYFYNATYENARTLVHYIKEYGLPFNIHYTPEDVDFTEEQIQAYIDSANNFFNDYPTLLFTRNHISKRDGTLKVRPVYAVDDLFIIIELMLTFPLTVQARKQSCCIMYGLETIRGSNHYIERLARSYSTYFSLDWSSYDQRLPRVITDIYYTDFLRRLIVINDGYQPTYEYPTYPDLDEHKMYTRMDNLLTFLHTWYNNMTFVLADGYAYRRTYCGVPSGLYNTQYLDSFGNLFLIIDAMIEFGFRTPEIDDFILLVLGDDNTGMTVISIDRIYDFITFLEIYALTRYNMVLSTTKSVLTTLRSKIETLGYQCNHGSPKRDISKLIAQLCYPENGLKPHTMASRAIGIAYASAGQDYMFHSFCQDVYNMFRLDYKPDARTTLNFQRQIYHNLDDGIPDLATPVVPPFPSIFEVREMYSRYQGPLTYAPKWNFAHFINSPDVTPAHYKTMRMYEIENNITIRPAPTFETVVPTTRNFP

SEQ ID NO: 47 - protein, 715 aa, Lentunula edodes partitivirus 1
MVFQQIRDFLSERKLRIQEEWRRYQKAVSRDDTELRQSDASDIRRMYEATRDQLTEQEKAAVLDREFQGLIDSMLLKNENKKENFEFSSTSAFDGFPPNRVPISGIAGIPRRFHTGQIVSESNEVPESGYPLDTLIDTLITNKYPEYRYYVDKYTRPLGTTDATFKDFNKEQVLIEPLDSARKERVMMHVHERLATTPYLPIHFVDTQFCKLPLHTGTGYFQRHSFWTQTHSKYSRPEEYHDRPTSKGYVMNAFLILARTAVHKIKVSGLPFDFDFDDFEDDNAAFNELAKYLDKFLNDHATMLFTRNHISQRDGKLKQRPVYAVDDLFILIEAMLTFPLLVLARDPACCIMYGLETIRGSMIYIDQISRMFNSFATIDWSEYDQHVPRPITDVYYYEFLPDLIVINHGYQPTYEYPTYPDLDEHAMYKRIDNLLFFLHFWYNNMTFVTADGYGYRRKHCGVPSGLFNTQYLDSFGNTYLIIDGLCEFGCSDEEIRLFLIFVMGDDNSLMSYWTLERLIAFIQFFEAYAKMRYNMTLSRTKSTLTAVRSKIEMLGYQCNFGRPSRPIGKLVAQLCYPERGLFRKFMSYRAIGVAYASAGIDVKFYKFCKDIYFTYLPYAVAASEFNFLRAATHLPGYLKAFDDVSDYINFEKFPTIYEIREVYSYYHGPLSYEPKWNRAHFINRPNIVPPSAKTVGDYRRENNLQPRQVPILPTD

SEQ ID NO: 48 - protein, 723 aa, Primula malacoides virus
MPFNAVRNYLAERLVRVRTELKNFTSSNREPDATLELSQDPDLRRYYDNTRYNSSNDAKYRTLNKEYSTLVEAYRTDNQQKHQPYELHQPIPADAAPIIDKRQPAPGLKLVPLMYHYGHVIHDPDPDQPDQTKVYPLDSRIYNLIMSTYPSYLSVLHDYCRPIGTVEATFNDFNTEQIPSAPIDEDRKQQILKHLFKFLDVKPYLPIHFVDTQYCKTPLVTGTGYHNRYSFKQRAHAKYSHPEEYALKSTSKGYFYNATYENARTLVHLIKTYGLPFNMQFACPKSDLTDEQINLYISKANQFFNDYPTLLFTRNHISKRSGTLKVRPVYAVDDIFIIIELMLTFPLTIQARKPSCCIMYGLETIRGSNHYIDRLARPYSTFFSLDWSSYDQRLPRVITDIYYTDFLRSLIVINHGYQPTYEYPTYPDLDEHKLYSRMDNLLYFLHTWYNNMTFLLPDGYAYRRTHCGVPSGLYNTQYLDSFGNLFLIIDAMIEFEFTDDEISKFQLLILGGDNTGMTNLAIDRIDKFITFLETYALARYNMVLPKTKCILTSLRSKIVTLGYECNYGSPKRDIHKLVAQLCFPENGLKAHTMSARATGIAYASAGQDIMFHSFCQDVYNIFRSDYKPDVRANLYFQRQFLNDLEEGVPDLATPTVPPFPSLYEIRKLYSKYQGPLSFTPKWNSAHFINEPDSVPISAKTMRQFEEEFSIPLMTAATFETVVP

SEQ ID NO: 49 - protein, 706 aa, Pleurotus ostreatus virus
MSFLRIRDYFTERLKHLSRDWKIFQQSDSDPESTLATHLDSDIARLEHGIKSSLSDDQRQQAYEREYNRIHSALHDKARQDGFPDEFYRSRSIDDLPDNRIPPSGIIPLPYEYHRSQVVISTEEVPETGFQIDPRIVRILRNKYPQYLPHVTKYVRPLGTTDATVKDFFKPQIPSDPIPEARKQRILDLVISFLACTPFLPLHFIDSLWDKTPLHTGTGYFNRHSFAARIHAMFSAPRLYERRTTSKGYFINYFLETARSTIHNIKLHGFPFDPSKVPDLGSALRSFILKRPTMLFTRNHISDRDGNLKQRPVYAMDDLFIRLESMITFPLHVMARKIECCIMYGYETIRGSNRQIDKIASSFRSFFTIDWSGFDQRLPRVITDIFWSDFLERMIVISHGYQPTYDYPSYPDLTPDKMFQRMDNILFFLHTWYNNLVFVTADGFAYIRTCAGVPSGLLNTQYLDSFSNLFLIFDGLIEFGCSDAEIYQIFLLVMGDDNSAFTLWSIAKLEEFLSFFESYALRRFGMVLSKTKSVITVIRGKIETLSYQCNYGAPKRPLAKLVAQLCYPERGPRAKYTSARAIGMAYAACAMDRTFHDLCRDIYYEFLDDSASPDEPFFFEHVQAYLPGILRTDESLSTQISLSSFPSFLTVQQHISRWQGPLSYYPKWDRAHFINDPDVIPPSAETMAEYRSRNSIPRRDIPSLWQ

SEQ ID NO: 50 - protein, 745 aa, Red clover cryptic virus 2
MPFNSARNYLAERMIRTKQELMTYQSEDHNPDAILEKSQDPDYRRYYDNTRFDPSNEVKHRILNKEYSTLVEAYRIKNDRKHQPYELHQPIPEDAAPIPESRVPAPGLKLVPLMYHYGHIVHDPVASESDSDDDNTASERPSKTSIPHFGYPVNKRIYDVIVNVYPEYLKVIGEYCRPIGTVEATFADFNKEQIPSAPINIERKEQVLTHIFKFLDAQPYLPLHFVDTQFCKTPLVTGTGYHNRYSFKQKAHAKYSRPEEYAKLPTSKGYFYNATYENARTLVHFIKQFGLPFNLQYAPEDADPTDEQVQSYIDTANSFFNDYPTLLFTRNHISKRDGTLKVRPVYAVDDLFIIIELMLTFPLTVQARKQSCCIMYGLETIRGSNHYIERLARSYSTYFSLDWSSYDQRLPRVITDIYYTDFLRRLIVINHGYQPTYEYPTYPDLDEHKLYSRMNNLLYFLHTWYNNMTFVLSDGYAYRRTHCGVPSGLYNTQYLDSFGNLFLIIDAMLEFGFSESEIDNFILLVLGDDNTGMTVISIDRIYDFINFLEKYALIRYNMVLSPTKSVLTTLRSKIETLGYQCNHGSPKRDISKLVAQLCYPENGLKPHTMAARAIGIAYAAAGQDPMFHSFCHDVYNLFRLDYKPDARTNLNFQRQIYHNLEDGIPDLATPVVPPFPSLYEVRHMYSKYQGPLSYAPKWNYAHFINDPDVTPPSPKTMRDYEIENDLISRTAPTFETVVPATRNFP

SEQ ID NO: 51 - protein, 709 aa, Rosellinia necatrix partitivirus 1-W8
MVLTIIRDYLHEAQLRLKKEWQTFQKSDQESGYSDKLPTDYDLRRYYDSARDYDAEKHKTEEYQHNFALTHERYTQMNADRNEPFEFYRPLEDNELPDIRFPAPGITVLPFRYHTGQIVETTDELPDSGFSLHPLIDYLTKTKWLHYRPYIDKYCRPLGTTNATFSDFNREQIPSAPIDETRKNMVLPLVIYFLNALPFLPIHFVDTRFCGTPKHTATGYFQRFSTFFRTHAYYARNKLYALRPTSKGYFFNTVYEFSRTWMHHIKEHGYPFVPSHDALDNARQYRIFMQKHVTMLFTRNHISDRDGFLKQRPVYAVDDFFILCELMISFPLHVMARYPINGIKSCIMYSFETIRGSNRYLDSIARDFISFFTIDWSSFDQRVPRVITDIFWTDFLRQLIVINHGYQPTYEYPAYPDLSEHDLYKRMNNLLHFLHTWYNNMVFVTADGFAYLRSAAGVPSGLLNTQYLDSFCNLFLIIDGLFEFGFTQAEILSIVFFIMGDDNSGFTMMDIERLTQFIEFFESYALKRYNMVLSKTKSVITTLRSRIETLSYQCNGGNPKRPLGKLIAQLCYPEHGPKDKYMSARAIGIAYAAAAMDEEFHEFCRDIYHTFLPYAAPIDEHTLSMATKHLPGYFKMLDNIASEIKFDSFPTLEMVQDKYSRWQGYLSHKPKWNDAHFKFLPETVPNNIKTMTDYQLEHKLDTPVPHSLF

SEQ ID NO: 52 - protein, 756 aa, Rosellinia necatrix partitivirus 6
MSSVFNNVRNYLQERLQHVKREWQIYQATGASSSNMTEERIQQLQDLDTRRMFHTARNAFDTTTNTPGPVSRITEEQRRLIYDAESDKIMNALRQHDEMRSQPFELFMTRPDSDPIPPNRIAAPGIWQSPLQFHTGQIVHADPSTTSVLHPDEHEDYAESYLPGDTDQGYEIDPTIYELLTRKYPEYLPYAQQYCRPKGTTDGTFRDFNKEQKPCPPLDSDRKEHVLKHVFKRLAIDPYLPLHFVDTQYCKLPLVTGTGYHNRYSYRQKAHASFSHPLQYGSRPTSKGYFYNATYENARTIIHNIKESGVPFNIHFAPEDRDITDTEIDEYRRKCNNFFDQYPTLLFTRNHISNRDGTLKVRPVYAVDDIFIIIEAMLTFPALVQARKPDCCIMYGLETIRGANHKLDSLAQSFSTYFTIDWSGYDQRLPRAITDLFYTDFLRRLIVISHGYQPTYEYPTYPDLNEDNMYDRMDKLLKWLHLWYNNMTFVTADGYAYRRMYAGVPSGLFNTQFLDSFANLYILIDGMIEFGFTDEEIDSFLLFVLGDDNSGMTNLSLARLHEFIQFLEAYALTRYHMVLSHTKSVITALRNKIESLGYQCNFGLPKRDIGKLVAQLIYPEHKIKYHTMSARAIGLAYASCAYDKTFYNFCKSIYNIFLDYYEYDEKTALNLSRFLTTGQDDLTTQFSWKILPPFPTREEIRKQISFYHGPLDYAPKWNFAHFINKPDVIPPSSKTMYDYEIEHSLRPQPAPTFVAR

SEQ ID NO: 53 - protein, 704 aa, Sclerotinia sclerotiorum partitivirus 1
MDSIRNIFQELRQTNVLEWKLFQKFGQTTTNPTYSHPDADLRRIQETFRDPHLERSQTQEFKLHYTDTYSGFRESDLSKNEDFEFYKPIDPSSIPASRLPAPGISVLPIRTHAEQVITATEQVPETGYQLHPLLRHLIVSKYPLYMQHATKLCRPLGTTDATFDDFNREQKQYPPIEPELALRIVRIIIHLLYALPFLPLHYIDTFFCKMPLHTGTSYFYRHSYELRTHVAFSAPSEYENKQTSKGYFFNAFTSWARTVAHRIKEFGYPFDPTQLTPSEITDKLRSFFMEHATMLFTRNHISDRDGALKQRPVYAMDTLFLHLEAMITFPLHVLARSSRSAIMYSMETIRGGCARMDALASSCQSYLCIDWSSFDQRMPWIIVDLFFTLFLPLLLIISHGYQPTAEYSEYLGLTPDKMFSRLFNIISFLRLWYYNCVFVTADGYAYVRRFAGIASGMLNTQYLDSFCNLFLMIHALLHFGCTNEEILDFIYFVMGDDNVILTQWTIDRLFSFLTFFESHSLSRFGMVISTKKSVITSLRSRIEMLGYQCNCATPKRPISKLVAQLCYPEHGPNDKYMSSRAVGMAWASAGYDAEFFAFCKDVHTLFTPFAAPPSEQTTQTILKHLPGLFKMLDDVTEFTNPQAFPDIMTVRNRYATWQGELSPDKKWSRAHFLRRPDDTPLPFQTMFEYMSEHGITFPEPQQLF

SEQ ID NO: 54 - protein, 746 aa, White clover cryptic virus 2
MPHNSTRNYLAERMIRTKRELMTYQSKDRDPDAILEASQDQDYRRHYDNARYDPSNEVKYRILNKEYSTLVEAYRLRNDRKHQPYELHQPIPEDAAPIPKSRVPAPGLKLVPLMYHYGHVVHEPAHSDESDSDDNSDAPTRPVKESVPHFGYPVSKKIFDLIVRVYPEYIKVINTYCRPLGTVEATFADFNKEQIPSAPINSKRRETVLKHIFKFLDTQPYLPLHFVDTQYCKTPLVTGTGYHNRYSFKQKAHAKYSHPEEYAKMHTSKGYFYNATYENARTLVHFIKQFGLPFNLQYAPEDADLTDDQVQSYIDAANNFFNDYPTLLFTRNHISKRDGTLKVRPVYAVDDIFIIIELMLTFPLTVQARKQSCCIMYGLETIRGSNHYIERLARSYSTFFSLDWSSYDQRLPRVITDIYYTDFLRSLIVINHGYQPTYEYPTYPDLDEHKMYSRIDNLLYFLHTWYNNMTFVLSDGYAYRRTHCGVPSGLYNTQYLDSFGNLFLIIDAMLEFGFSDAEIDDFILLVLGDDNTGMTVIPIDRIYDFINFLEKYALVRYNMVLSPTKSVLTTLRSKIETLGYECNHGSPKRDISKLIAQLCYPENGLKPHTMAARAIGIAYAAAGQDFMFHSFCHDVYNIYRLDYKPDARTNLNFQRQIYHNLEDGIPDLATSVVPPFPSLYEVRHMYSQYQGPLTYAPKWNYAHFINDPDDVPPNPKTMREYEIENDLISRTAPTFETVVPATRNFP

SEQ ID NO: 55 - protein, 621 aa, Amasya cherry disease-associated mycovirus
MDHLTSLFELFAITPKTQNNLQFVGIYHRPPHSVRANLRNVEKHKITVAHAMHKYLYPHEIDFVINQMRRSDVTEDAILADFFDNNVEPLEPVLDEHFERGLSAMLDAFRPPQKCLPAHIYDVQHHYPYKWQVNAEAPFSTDSYFLANRPTFRAVFERLESLYTHLATDWHRRYGNKTDNDDFMNDHVPAKFGPMKETVFSWTHRWHHVIKSNFTDTAGLSKDYYFKNRYIFPMLLHTKTAIVKKDDPNKMRTIWGCSKPWIIADTMLWWEYVAYAKLQPGATPMLWSYETFTGGWLRLNHALFSSYIRHSYITLDWKRFDKKAYFCIIDKIFDGVETFLDFDNGYLPTKDYPDTKSTWTQERSTRLKRLFDWTKENFYHAPIVLPNGHMYVRKFAGIPSGLFITQLIDSWYNYTMLATILSAMGFDPRSCIIKVQGDDSIIRLSALIPPDAHDSFLTKVQELADYYFQSVVSVNKSEVRNELNGCEVLSYRHRHGLPYRDELAMLAQLYHTKARNPSPEITMAQSIGFAYASFGNHERVRLVLHDIHEYYKLQGYTPNRAGLSLVFGNSPDLMIPHYTLDHFPSLREIKMFLTNAEYVNEETNSRTWPLNHFLHLPCHRT

SEQ ID NO: 56 - protein, 585 aa, Arabidopsis halleri partitivirus 1
MKNTVVLEPLPSLARPIYGDTDPGRNPAYQSTVDHALRRLLTAEEFNIVVNGYRRSPWNEDALTADIEKLNSDYHHVNKDEHYYKAIEHTKKLFTPKEKLRPVHFNDLRHYPWQLSTSIGAPFATSEKWKDYINQKYDGKLKSRDFKDLFKETHGVSLEPYMIDRRLSKRNFYNEMFYINRINIHHIKDGWTTNPAGHDLRYWHTAHARQHLVEAGDEDKVRLVFGAPSTLLMAELMFIWPIQTSLLARGSSSPMLWGYETTTGGWSRLYNWAYSALPRFGAVATLDWSRFDKDARHTVITDIHDLIMRPMFDFNSGYHPTIINPRSNPDPQRLENLWNWMKNAILTTPLLLPDGTRLQFQHSGIYSGYFQTQILDSMYNCVMIFTVLSRMGFDLNSVAIKVQGDDSLILLSHSYTFLQHSFLTTFAHHAAVYFGSTLNVKKSELLPSLEDAEVLRYRNHGMMPYREELQLLAMLRHPERTASLSALMARSIGIAYANCGNYTRVHHICEDIHNYLKGIGVKPDAFGLPGGLRFRKNYLPSYEEIDISHFPTWLETVERLLDPSRPLLTNKHWPTTHFFGIPGES

SEQ ID NO: 57 - protein, 616 aa, Beet cryptic virus 1
MDYLTSAFNRITHWFNVPSNLEYIGTFSLPPGILRVNEVAISNHKKTLEHSFNKYLYAHEIKLITQDYRRSDIDEESILADFFSGDVEKFEVPFDEHVETGLRCMADAFRPPRLCRPAHILDVKHGYPYKWNVNAEPPFSTDEYFLNQRKTFGEFIRMHEYEHIDKADFFRRHPNTESHDLIRTIVPPKFGYLKSMIFSWTRRWHHIIKEGFTESTGLHTTGYFYNRFIFPMLLHTKTAIVKQDDPNKMRTIWGASKPWIIAETMLYWEYIAWIKLNPSVTPMLWGYETFTGGWFRLNRDLFCGFLQRSFLTLDWSRFDKRAYFPLLRRILYTARTFLTFDEGYVPTHSAPTHPQWDHTKAIRLERLWLWTLENLFEAPIILPDGRMYRRHFAGIPSGLFITQLLDSWYNYTMLATLLSALGFDPKHCIIKVQGDDSIIRLNVLVPQDQHQNLMDNLVQLAVNYFNAVVNVKKSEFGNSLNGREVLSYRNHNGFPHRDEIMMLAQFYHTKARDPTPEITMAQAIGFAYASCANNKRVLWALKDVYDYYKDLGYTPNRAGLTLTFGDSPDLFVPEISLEHFPTETEIRRYLTSTSYLNEAQNARTWPRTLFINAPAQ

SEQ ID NO: 58 - protein, 616 aa, Carrot cryptic virus
MDYLTTAFNRITHWFTTPTDFEYIGNFSLPPGLLRVNDTAISNHKKTLEHSFSKYLCADEIHLITKEYRRSDIDLDSILDDFFSGDVEKFEIPFDEHVETGLRCMADAFRPPRLCRPAHILDVKHHYPYKWNVNAEPPFSTDEYFLTQRKTFGEFIRMHEYEHIDKADFFRRHPNHESHDLLQTIVPPKFGYLKSTIFSWTRRWHHVIKDGFKDSSGLQTNGYLYNRFIFPMLLHTKTAIVKHNDPNKMRTIWGASKPWIIAETMLYWEYIAWIKLNPGTTPMLWGYETFTGGWFRLNHQLYCGMIKHSFLTLDWSRFDKRAYFPLLRRILYPVRSFLTFDEGYVPTHAAPYHPQWNHEKALRLERLWLWTLENLFEAPIILPDGRMYRRHFAGIPSGLFITQLLDSWYNYTMLATILSALGFDPKQCIIKVQGDDSIIRLTVLIPQETHERFMAHLTHLATYYFNAIVNVKKSEVANTLNGREVLSYRNHNGFPHRDEINMLAQFYHTKARDTTPEITMAQAIGFAYASCGNHNRVLWVLNDIYNYYKDQGYSPNRAGLTLTFGDSPDLFVPEIPLDHFPTKKEIRRYLTASSYINEAQNARTWPRDLFINRPAE

SEQ ID NO: 59 - protein, 616 aa, Dill cryptic virus 1
MDYLTTAFNRITNWFTKPANFEFVGLYSLPPGLLRVNDTAISNHKKTLEHSFSKYLYEDEIHLITKEFRRSDIDLDSILDDFFSGDVEPFEIPFDEHVETGLRCMADAFRPPRLCRPVHILDVKHHYPYKWNVNAEPPFSTDEYFLSQRKTFGEFISMHEYEHIDKADFFRRHPNRESHDLLKTTVPPKFGYLKSVIFSWTRRWHHVIKDGFKDDTGLKTTGYFYNRFIFPMLLHTKTAIVKHNDPNKMRTIWGASKPWIIAETMLYWEYIAWIKLHPSVTPMLWGYETFTGGWFRLNNQLFCGMIKHSFLTLDWSRFDKRAYFPLLRRILYQVRTFLTFDEGYVPTHAAPYHPQWDHEKALRLERLWLWTLENLFEAPIILPDGRMYRRHFAGIPSGLFITQLLDSWYNYTMLATILSSLGFNPKQCIIKVQGDDSIIRLTVLIPHENHLKFMERLTELATYYFNAIVNVKKSEVANTLNGREVLSYRNHNGFPHRDEINMLAQFYHTKARDPTPEITMAQAIGFAYASCGNHKRVLWVLNDIYNYYKDKGYTPNRAGLSLTFGDSPDLFVPEIPLDHFPTTKEIRRYLTCSSYVNEAQNARTWPRTLFLKDPAE

SEQ ID NO: 60 - protein, 621 aa, Diuris pendunculata cryptic virus
MEYLIADFSRITHFFRDTTNLTYSGTYHFHPRHPEVNLNAYEAHQRVLRSLMDTHLFPHEIQLITDELRRSDMTIEAILADFFANDVEYHEIPFDSHIEYGIKCMLDAFRPPKRCRPVHLLDVQHHYPYKWQVNAEPPFSTDTYFLDNLPTYRDFWNERTSSFDKYVDPEELNRRLRHRNIENLLDTKTPAKFGFLKNTVFSWTRRWHHIIKDGFTDTTGLTSDAYLRDRFIFPMLLHTKTAIVKKLDPNKMRTIWGVSKPWIIAETMLYWEYIAYVKQNTGATPMLWGYETFTGGWLRLNAALYTSHVRFSFLTLDWKRFDKKAYFPLIYKILLGVRDFLDFDNGYAPTVDYPDTKSTWTPHKSQRLQNLWLWTIENLFNAPIVLPDGRMYKRRFAGIPSGLFITQLLDSWYNYTMLASLLSALSMNPKSCIIKVQGDDSIIRLGTLIPPSQHEAFLLKLHALADFYFKASLSLDKSEVRNSLDGCEILSYRHIRGIPYRDEITMLSQFYHTKARNPTPEIAMAQAAGFAYASCGHHRRVYNYLESVYNHYAVQGYTPNRAGLSLVFGNSPDLILPHFELDHFPTISEIQHYYTSSVYRNESQMSKIWPLDHFLYPPAET

SEQ ID NO: 61 - protein, 620 aa, Heterobasidion partitivirus 7
MEYLSNLFSRVLKITKTTNFEFVGTYHNQPSIPQVNQIAIDNHQRTIRVAMERYLTSDEFSLITTGYKRTSLDPATITDDFFSGDIEPHDEPQDLASQLAIEAGLNAMQRAFCPPNPARPVHLYDVEWHYPYKWQVNAEVPFSTETYFLKLRKKFSDFYDAASKTWTHYVNPLDALRRYGPEPPFDTLNQVTPPKFGFMKELIFSFVHSWLHVIKSRFHSNAGYTHSNFLRQRFLFPMQLHIKTALVKADQPNKLRSIWGVSKLWIIAETMIYWELIAYMKLNRGSTPMLWGYETFTGGWFRLNAELQSSHLRQSIITIDWSRFDKRAYFWLIRKILFRIRQHLDFNNGYVGTKDYPSSPTDPDKLQALWEWTLEALFDSPIILPDGKMYKRRFAGIPSGLYITQLLDSWYNYTMLAAILTYLGLDPERCIIKVQGDDSIIRLYVLIPPSEHDNFLLKMQEVATHLFASRISDQKSEVRDDLNGAEVLSYRNNRGLPYRDEIQMVAQFYHTKAKDPTPEITMAQAIGFAYAACGNNYRIHSLLEEVYNYYHEQGFTPNPAGLSIVFGDSPDRPDYPIELDHFPTQQETQRFLLSTDYRNAEQDARTWPLLHFLHAPCSRS

SEQ ID NO: 62 - protein, 621 aa, Heterobasidion RNA virus 1
MDYLTGLFSRVLHISRKVTNFEFAGTYHYQPSIPQVNEVATENHKRTLRHSFRTYLTSDEYDKIVNGYKRTNLDPSTITEDFFSGDIEDHPEPTDFKSQLSIEYGLQCMIDAFKPPAPARVCHLYDVQWHYPFKWQVNAEAPFSTEKYFLDLRKKFGDFFDPVTKLWTKYVNPLDALRRYGHTPPADTLNQVTPPKFGFMKNLIFSFVHSWQHVIKSRFTSNAGITHSNFLRQRFLFPMLLHIKTAIVSFDAPNKLRSIWGVSKLWIISEAMIYWEYIAWIKLNPGSTPMLWGYETFTGGWFRLWRDLHTPGEDVTYITIDWSRFDKRAYFWLIRKIFIRTRCFLDFTNGYVSTKDYPTSPTDPDKLQALWEWTIEAFFDSPIVLPDGSMFKRLFAGIPSGLFITQLMDSWYNYTMLAAILHYMGYDPRRCIIKVQGDDSIIRLYIQIPLHEHDLFLLRMQEVSDHLFGAKISFEKSELRNSLIGSEVLSYRNIQGLPYRDLIKMLAQFYHTKAKDPTPEITMAQAIGFAYAACGNDFRIHELLRSVYDYYKAQGFTPNPAGLTVVFGDSPDRPDYPISLDEFPSQMDVQRFFLSTDYRNADQENRTWPSSHFLYAPCSRI

SEQ ID NO: 63 - protein, 616 aa, Red clover cryptic virus 1
MDYFISAFNRITHWFTTPTNFEYVGNYSLPPGLLRVNDVAIANHKKTLEHSFNKYLYSHEIKLITKEYRRSPIDEDSILEDFFSGDFEYFEIPFDYHVEYGIQCMADAFRPPRLCRPVHILDVKHGYPYKWNVNAEPPFSTDEYFLSQRKTFGEFIRMHEYEHIDKDDFFRRHPNSESHDLLRTIVPPKFGYLKSMIFSWTRRWHHIIKSGFTESTGLETNGYFYNRFIFPMLLHTKTAIVKKNDPNKMRTIWGASKPWIIAETMLYWEYIAWVKLNPGVTPMLWGYETFTGGWFRLNNELFCGLIKKSFLTLDWSRFDKRAYFPLLRKILHTTRTFLTFDEGYVPTFAAPTHPQWNQEKAHRLDRLWIWTLENLFEAPIILPDGRMYKRHFAGIPSGLFITQLLDSWYNYTMLATILSALHFDPKHCIIKVQGDDSILRLTTLIPVDQHANFMSHVVRLASHYFNSIVNVKKSEVANTLNGREVLSYRNHNGLPHRDEITMLAQFYHTKARDPTPEITMAQAIGFAYASCANHNRVLWVLHDIYNYYHDLGYRPNRAGLTLTFGDSPDLFVPEISLDHFPTKSEIRRYLTALHYQNEAQNARTWPRTLFINAPGE

SEQ ID NO: 64 - protein, 620 aa, Rhizoctonia fumigata partitivirus
MEYLVHAFKMFSLEPSVPQNLQMIGTYHYKPASPHVHTPHVEAHKKTVLKAMEKYLYPREINTIVNELRRSDVNLESILDDFFDNNVPLHRIPFDEHVEYGLQCMADAFRPPRPARPCHLNDVEHHYPYKWQVNAEAPFATDKYFLDNRHKFRDFYDETTGTWKHADPIDMERRYGNRLDTVLDQITPPKFGYMKNAIFAWTRRWHHIIKDGFTDLTGLVSSTYVRDRFIFPMLLHTKTAIVKKDDPNKMRTIWGCSKPWIIAETMLYWEYIAWVKLNPGITPMLWGYETFTGGWLRLNAALFSSLMKNSFVTLDWKRFDKRAYFELIYRIMLVARTFLDFENGYVPNVNYSATHTDWSHTKAQRIQRLWEWTLDNLFNAPIVLPNGDMYKRNWAGIPSGLFITQLLDSWYNYVMLATLLSALGHDPKACIIKVQGDDSVIRLAVLIPPQYHELFLTRLQDLADHYFGAVISIEKSEVRNELNGVEVLSYKHIHGMPYRDEIAMMAQLYHTKARNPEPSITMAQAVGFAYASCGNHDRVYYALKDIYEYYASQGFAPNPAGLTLVFGNSPDRFELDVPLDHFPTIHEIKKYLLNFEYRNEHQEHKTWPLDYFLNPPCSTL

SEQ ID NO: 65 - protein, 603 aa, Rosellinia necatrix partitivirus 2
MKNITDLPFPSKGVPQFRDALPIHGRDPEVPNPVTDASNRIIDFALRKHLTSDEFDQVVNGYRRSPWNEDALNKDIEKLDSDEHTVIKDQHYENAIQHVQKLLTPEKPLQPVHFADLRRYKWRLSTNIGAPFASSKHWQDYVKAKFNHFRDGTPFENIAHRDLFVEAHKDSQPLEITDARMTKHNLYTEAFYVSRETIHRIKDGETTDRYGNDARYWNTAFARQHLVKADEDDKVRLVFGAPFSLLCAELMFIWPLFIFLLSLKGSTAFMLWTFETIIGGWYRLVNFFTTYALRHSTVVTVDWSGFDRYARHTVIKDIHHRIIRPMFDFSHGYHPTRDYPDTSKTKDGQSNEWRITNLWNWMTDAILSTPLLLPNGRFIRFNHSGIYSGYFQTQILDSIYNLVMIFTILSRMGFDLDKCVIKVQGDDSIFMLLCCFIMISTSFFTLFKYYAEYYFGAKLNEKKSEIRPSLQDAEVLKYRNRNGIPYRDRISLLAQLRHPERRTDVDSVAARCIGIAYAACGQDATVYLICEDIYNYLVKKYDAHARQGELDFMFRHLELHDTVPSAATFPSWFDTMAHLTDGPRDPVPSHWPTDYFIGLPGRL

SEQ ID NO: 66 - protein, 528 aa, Rosellinia necatrix partitivirus 7
YNVQTAIDAVTEQFRPNRVLHPIQYPDLRYYPWTLNVSAEAPWTSYNFHFVPMDRSVDFESTQPKLIFDINQVNKLRKFSKPTDVKTYLRWKQQVGLIENDHITFHNLYDEIFIYNRPLIHQIKEGEEPFWKDGQPVPYLWNTLHVRSHVVAHNEPDKLRGVFGATKLVLQTEQPFIWPLQASYLNTDAGRLLWGREMSKGGWRKLFSEIYTFGPPSTVLSTDWSQFDKRLLHQLIRIVHRIWRSYFDFTRYEPTNQYPNANPRDPKRLERLWDWMCNAITDTPILLPNGEIWRWQWNGFGSGYQQTQLMDTFANAIMIYTCLTALGVDVTNPKFWARFQGDDSLVAFFEQMFRIYGNDFLIMFSAVAEKYFNAKLNVKKSSILGQAHMATVLSYPNWHGTAFRTDEDLLRHLMFPERPQDLGRLAASAIGLAQAALGCSERFHNLCEYIFTKLVKGKGVKVKWQALKWMVRAGQFETIEQLKRTEFPTIEELLSQAQIPAIRTESERQRIWRTTPLSKDSFHFTHDI

SEQ ID NO: 67 - protein, 586 aa, Rose partitivirus PB
MRNTVVIGHRPVLAKPLFGNPDPGSNPAYGDTVDHALKRHLSPEEFDIVVHKYRRSPWNEDALKDDIAKLDSNEHPVLKDEHYYRAIEHVKKLFTPEEKLRPVHFADLRYYPWQLSSNIGAPFATSKKWQEYVNDKFTAGQTAPQVRNLFQEAHGTPLEPEVIDRRMTKRNLYNEMFLINRKNIHLIKDGRKTNDSGHDLRYWHTAFARQHLVEHDEPDKVRLVFGAPSTLLMAELMFIWPIQVSLLARGPDSPMLWGYETLTGGWSRLHTWATKAQPRLGSVLTLDWSRFDKDARHTIIKDIHSLIMRPMFTFDNGYHPTVYYPETPETDPNRLENLWNWMTDSILTTPLILPDGKILQFMHSGIYSGYFQTQILDSMYNSVMLFTILSRMGFNIEKVAIKVQGDDSIILMPYQYTVIKDTFLQFFSAYAQEYFGSTLNMKKSEILPSLERAEVLRYRNHGTMPERDELQLLAMLRHPERTSSLPSLMARAIGVAYANCGNHTRVYQICEDIYKYLAKGGFKPDPFGLPGGLRYRQNYIPSYVQIDISHFPTYFETVMHLQDPHRAILTNRHWPTDHFIGTPGKS

SEQ ID NO: 68 - protein, 623 aa, Rhizoctonia solani dsRNA virus 2
MNLYNRVSALFANWFSSPSNLEFVGSYHHQPGTVPPNPSTQEAHKRFLHNVFKQHLFTYELDYIENEHRRSEATPEAIENDFFANDVEPHDIPFDVHVEIGLQCMTDAFRPPVPCLPAHLNDVEHHYPFKWQVNAEPPFSTDSYFLDNRKLFSDYYDTESQTWRGYVDPFEANRRYQHTKDKEGFLNQTVPAKFGFMKDTIFSWTRRWHHIIKEGFQTATNLSSTAYLRDRFIFPMLLHTKTAIVKKDDPDKMRTIWGCSKPWIIADTMFYWEYIAWIKKHPGVTPMLWGYETMTGGWMRLNSQLFSSYLKKSYVTLDWSRFDKRAYFKLILAIMCRVRTFLDFDNGYLPNVNYPDTRTDWSPNKAQRLERLWLWTLECLIKSPIVLPDGRMYIRHYAGIPSGLFITQLLDSWYNYTMICTILSSIGLNPKHCIIKVQGDDSIVRLGVLIPPEAHEAFLLALQSKADFYFKAIISVDKSELGNSLNNREVLSYRNYNGLPRRDEIKMLAQFYHTKARNPTPEIAMAQAVGFAYASCGTHQRVLDALEHVYTDYKDAGYTPNRAGLSLVFGNSPDIQLPHYDIDHFPSIEEIKRFLTCNSYDNSVQMAKSWPTSYFISEPCERM

SEQ ID NO: 69 - protein, 616 aa, Spinach cryptic virus 1
MDYLTSAFNRITHWFTKPTNLEYIGDYSTPPSIIRVNEVAIANHKKTLRYAFEQYLYDHEYRMIVDEYRRSDIDQESILADFFSGDIEPFDIPFDEHVEHGLRCMANAFRPPRPCRPAHILDVKHGYPYKWNVNAEPPFSTDPYFLSQRKTFGEFIQMHEYEHIDKADFFRRHPNTESHDFLTTVVPPKFGYLKSMVFSWTRRWHHVIKTGFQDLTGLENSGYLYNRFIFPMLLHTKTAIVKKNDPNKMRTIWGAPKPWIIAETMLYWEYIAWIKLNSGKTPMLWGFETFTGGWFRLNNILFCGLIKQSFLTLDWSRFDKRAYFSLLRRILYTARTFLTFDEGYVPTHAAPTHPKWNHQKALNLERLWLWTLENLFEAPIILPDGRMYRRHFAGIPSGLFITQLLDSWYNYTMLATLLSALGFDPKYCIIKVQGDDSIIKLTTLIPRDQHENFMVQLTALATTYFNATINVKKSEIRNTLNGCEVLSYKNHNGFPHRDEITMLAQFYHTKARDPTPEITMAQAIGFAYASCGNNKRVLWILRDIYNFYKMRDVTPNRAGLTLTFGDSPDVFMPEIPLDHFPTITEIRRYLTCSEYRNEAQDARTWPRSLFISGPAE

SEQ ID NO: 70 - protein, 584 aa, Sophora japonica powdery mildew-associated partitivirus
MASTNLLRLGKIPRSKKHNDPLSRSLRINRIRQGIIKRAIYKICPINLARQVIFGFKRSEGSDDVAETDFLRSDVPYFDMKRDFHYLRALRVCERLFRPSRTLHPIAFPDLRFYPWSLSVSAEAPFSVEKKRSTLIRSRQSDGEDLDGRLTFHNLYNEIFELNRNLIHQIKEGDKSFWNKDGTPRPYWYNTLHTRPHLVKSSEPDKLRAVFGVPKLLLMAENMFIWSLQKEYLNQKIQSPMLWGFETFKGGWLKIWNRMYSKKCSTFLSADWSGFDRFALFECVDDIHRMWRNWFDFSKYEPTIAESGPPGIQLSYPKSKTNPQKIERLWNWMCYCSKYTPIKGQSGQLYQWQYNGIASGYQQTQLIGSFVNSIYMLTCLSDLGINIESDNFQLFVQGDDSLTEFSEIIQKDDLPKFLTNLAKVAKSRFNANLSVQKTTAGESLNDVEVLSYNNTYGIAFRDEAELLAHLLYPERFQTLEATVSCCIGIAYASMGCSQYVDDTCLDAYNFLTTQFKVKPDSNFLQDFFRIRGSPLTDDFHNPRFPTKDECFNQNYEVRSRSDSEKQRLWPSIPTGDYGFHFINE

SEQ ID NO: 71 - protein, 580 aa, Sclerotinia sclerotiorum partitivirus S
MPALNLRYLFSISRSEMKKIRSSHTKVSPREEATRFSILKHAILKHGSVGLLNQVLLGKRRSDASDERLIQDFHEFEQPVHPVPRDKHYLRALRVTEKLMKPAKTLHPISFPDLRYYPWTKNVSAEAPYNFEKKYEELLRDKQRLGEIETATATFHNLEDEIFEDNRYLIHKIKEGDSQFWDKDGKPRPYYHTTLHARAHVVGHEDADKIRAVFGVPKLLLMAENMFIWPLQAYYLNQDTTKHHLLWGNEIMKGGWKKLWGQLQNGRISRTILSLDWSEFDKRALHEVIDDVHSMWKSWFDFTHYEPTIFYEKGEIPEPHRRIENLWIWMTDMVKHYPILQPDGKVYQWTRNGIASGFQQTQLLDSFVNMIMLLTVLSANGINIEHPDFWIKVQGDDSLISIVERRFQMFGISYLDTLADLASYYFNAKLSVKKSFISDTPQGQYVLGYFNHYGIPYRLDDDLLSHLVFPERPQRLEETAASCVGIAMASMGCSKVVYSICDDAYTFITKTLRRPAKAGSLFWLERAFGYEMPNIAKMPTFEECLYASYDIPVRTENMKQRLWPTNAKAKNGFYFLRHLC

SEQ ID NO: 72 - protein, 616 aa, Vicia cryptic virus
MDYLISAFNRITHWFITPTNFEYIGYFSLPPGLLRVNDVAIANHKCTLERSFHTYLFDHEIKRIMIDHRRSDITEDSILEDFFAGDFPYFEVPFDEHVEYGLQCMADAFRPPRPCRPAHILDVKHGYPYKWNVNAEFPFSTDEYFLTQRKTFGEFIRMHEYEHIDKDDFFRRHPNLESHDFLRTIVPPKFGYLKSTIFSWTRRWHHIIKLGFTDTTGLDNNGYLYNRFIFPMLLHTKTAIVKKDDPNKMRTIWGASKPWIIADTMFYWEYQAWVKHNPGSTPMLWGFETFTGGWFRLNQLLFCGLIRRSFITLDWSRFDKRAYFPLLRKIMYTVKSFLTFEEGYVPTHAAPNHPQWNQDKTDKLERLWLWTLENLFEAPIILPDGRMYRRHFAGIPSGLFITQLLDSWYNYTMLATILHALGFNPSNCIIKVQGDDSIIRLNVLVPSERHDHLMSRIVELAEYYFNSIVNVKKSEIRNRLNGCEVLSYRNHNGLPFRDEIAMLAQFYHTKARDPTPEITMAQAIGFAYASCATHTRVLWVLEDIYNYYRDQGYTPNRAGLTLTFGDSPDLTMPEMPLDHFPTKSEIVRYLTCTNYRNEAQNARTWPRTLFINAPAE

SEQ ID NO: 73 - protein, 616 aa, White clover cryptic virus 1
MDYLITAFNRITHWFLTPTNLEYIGSYSLPPGLLRVNDVAVANHKATLDRSFDKYLYEHEINLITKEYRRSPIDEDSILEDFFSGDLPYFEIPFDEHVERGLECMAAAFRPPRPCRPAHILDVKHGYPYKWNVNAEPPFSTDEYFLSQRKTFGEFIRMHEYEHIDKEDFFRRHPNIESHDFLRTVVPPKFGFLKSMIFSWTRRWHHIIKSGFQDSTDLEQTGYFFNRFIFPMLLHTKTAIVKKNDPNKMRTIWGASKPWIIAETMFYWEYLAWIKHNPGATPMLWGYETFTGGWFRLNHELFCGLIQRSFLTLDWSRFDKRAYFPLLRRILYTVKTFLTFEEGYVPTHAAPTHPQWSQENIDRLERLWLWTLENLFEAPIILPDGRMYRRHFAGIPSGLFITQLLDSWYNYTMLATILSALHFDPLHCIIKVQGDDSILRLTTLIPVDQHTNFMDHIVRLADTYFNSIVNVKKSEVRNSLNGCEVLSYRNHNGLPHRDEITMLAQFYHTKARDPTPEITMAQAIGFAYASCANHNRVLWVLEDVYNYYRDLGYRPNRAGLTLTFGDSPDLTMPEMPLDHFPTKSEIRRYHTETHYQNEAQNARTWPRTLFINAPGE

SEQ ID NO: 74 - protein, 571 aa, Alternaria alternata partitivirus 1
MLLSEIPNVYQRALALERRLRLRELYPEGIIPYLARQEYAGSIAGPRGPSATVAPSVTTSFIAKAPLSTYSKPEYTLECTETMQYLGKMDHYAFNNSVPKFDPWFRHVLKSKAPDVVLHLEETYQRDPCTPERVMKFMKLFDRRWKRMPTGNVMKQAKEIVSEMFSKVGKVDPIDFNYAGWHEILPHLDMSSSPGLPLRREYATQGECLGHIYDKTKRLNHFAKFLHPGAVRAPPCMIGLRPGLIKKAEIDEKIKARGVWAYPAEVKVIEMRYCIPLMKRFSEMFGKTPYPVGRNMTKALPFIIDHLLQDKKFGLVTDISKLDTSVGPDWIDWAFSQLKSFFDFGFTLSSERRDSNVFDFLHFYFKRTPILLPSGQLVKKAGGVPSGSGFTQLVDTLVTTLATVYSRLRMGHTKDQIFKMFVVGDDMATSVDHDFSIEEFSHIMGQLGFEINPSKVMFSNKGIELKFLGYSKRGGGLYRPIEELLQTAFFPEKYVGNPNRSRQRILGQSIAAGLSNSFFDKCNYWMEELVTLSSTLDPDEVFIPQKRWMRNVLSIDEMPKSANVYDLFHLV

SEQ ID NO: 75 - protein, 475 aa, Beet cryptic virus 2
MRTINNYEYTSFTEDLEETDYTHPHVVRRDPEVTYEDTFAKKELLSRYPALYENLIRGWSRSYYTGQEHLRAIMQYATPNTNFSECVQHAYTKAITKVTESLHSLPTVRAFNVLEELDLIPYESSSSAGYTYRGVKGPQHGENHMQAIKTAKAVLWSVIKDDGEGIEHVIDTYVPDVGYTRTQLTDLREKMKVRGVWGRAFHYILLEGTSAAPLLEAFANSNTFFHIGKDPTVSVPYLISYTKGQAPWLTAIDWQAFDATVSRFEINAAFDIIKSKITFPNLETEQAFEISRQLFIHKKLAAPNGKIYRIHKGIPSGSYFTSIIGSVVNRLRIEYLWNLKFNRGPKVCFTQGDDSLIGDDELYSPMDMAAFVKPLNWFINTSKSMTSKVPEAITFLGRSSLGGLNQRDLKRCLRLLILPEYPVTSGDISAFRANSIWRDSGSTSQILHEIANALRRKYGIAKEQDVPRYLRPWKA

SEQ ID NO: 76 - protein, 572 aa, Botryosphaeria dothidea partitivirus 1
MLLTEIPNVYQRALALERRIRVRELFPEGLVPYLKRQEHAGSIASPIGPSRSVAPKVTDKFIAKAPLEDYPDKRYTLECTSTMQYIGQMDAYPFHSSIPEFDPWFRHVLKARAPDVSLHLEQTYTRDPCTPDRVMSFLKLFDRTWKRKPTGRLMSQAESIVKKMFKCVGQVNPIDFNYAGWHEILPHLDMSSSPGLPLRREYATQGECLGHIYDKSKRLNHFAKFLHPAAVRAPPCMIGLRPGLLKKDELDEKIKARGVWAYPAEVKVIEMRYVIPLLERFKSQFGKIPYPVGVNLTKALPFIIDHLLNDGKHGFVTDVSKLDTSVGPDWIDWAFSFLKDFYYMGMTESSETRNSHVFDFLHYYFKRTPILFPSGQLVRKSGGVPSGSGFTQIVDTLCTLLMTTYSMLRMGYQEDDIIGKIFAVGDDMATSVPSSFDVEQFSFYVGQLGFEINVDKVMFSNRGIELKFLGYSKYGGNIWRPIDELLQTAYFPEKYVGNPERSRQRILGQTLASGLTNGFLSKVNYWMEELCSWHTELDTDEVYIPQKRWMRNVLGLDEIPRSALLFDIFPLC

SEQ ID NO: 77 - protein, 415 aa, Citrullus lanatus cryptic virus
MNISPLLYEQCLSGWSRSYYLHDKHMQAIIQYGYKDVPISSINDRLYKECVHEVQNRLSSLPRVRALDVLSELDSVTFKSSSAAGYDYIGAKGPKGGENHTRAMSRAKAIMWSIAETGETGMKHAIETAVPDVGYTRTQLADITEKTKVRHVWGRAFHYILLEGLTADPLIRAVQRADTFIHIGKDPTVSVPRLLSDTAEQCKWLYALDWKQFDATVSRFEIEAAFDIVLNLLDFPNRETKLMFELSKQLFIHKKIAAPDGKIYWAHKGIPSGSYFTSIIGSIINRTEILMLWRTITGHGPIVCYTQGDDSLCGDNILIPPERFAMVANPIGWYFNQEKTEYSTIPELITFLGRSYAGGLNKRDLKRCLRLLIFPEYPVESGRISAYRAQSISDDVGGLGDVLNKLADRLRRSIP

SEQ ID NO: 78 - protein, 491 aa, Diatom colony associated virus 14
MQEMTYSGVRYRTSNQGDYITKHPFGLSQIGSHKFTPVRRDPHTTIIDPFMMEAFQDYGDRFNFAKLDGWSRSLYTREGHMDSIHRIQSHTRFHKPTDTSMTKTDDYCLQIFRTLGTVRSLDYHTQLGQVPFEPNSAAGIGIPGKKGDSGNLALAINQAVATLQRSLRDGISSVIEDSTPDMAYTRTQLTQLSAGIKVRNVFGQAFQYILLEGLSASPLMDHFVTNETFFFVGSDPRISVPTLLEDFKKKGSLMMSIDWSAFDTSVENWEIVDAFNLLETILEFPNLETRAAFEFSRILFINRKIAAPDGNVYFKQKSVPSGSYYTMLIDSIINWRRILYLHHRATGFFPFDIRTQGDDSLVATRDSVSPEALMLQIPRNSQWQLNPSKCPIGKSGSSVPFLQRTLKWGDQSRDLDRVERLAIYPEYEVESGDISAFRARALWEDCNYESVVLAHATSYLESKYGIPTTVPRRYTNIWQTLFESKEREGLR

SEQ ID NO: 79 - protein, 479 aa, Fragaria chiloensis cryptic virus
MEHRFRGIPRGLIELEEIPTRRLREERVVHIDAWSSKAIDAIVPLSLRIELDGWARSYYTLQAHIDSIMQYDRPKLPQPTNAAWNTTTQHVRTQFARMDKVQTLSYLQLDQVKWVRSSAAGYGYVGRKSDNDNYFRARKTAFTIAEKLNHDRDYGPLALEDSTPDIAFTRTQLCQIKVKRKIRNVWGEAFHYVLLEGLFADPIIQHFIRNKSFYFIGEDPLLAVPRLVEKILSEQDYVYMFDWSGFDASVQEWEIRFAFSLLESILIFPSSVESYIWHFIIELFIYRKIAAPNGKVYLKTLGIPSGSCFTNIIGSIVNYVRIQYLFFRLTNNFVTVFTHGDDSLVGVSTTQYVQMDNFEPICAEHNWTINIAKSAVSHEAEGVSFLSRKVREHCHARDELLCLRMLKFPEYVVESGAMSTLRAHSIHQDAGINSRYLYSIYKYLLHRYGKADSLPLNQQNWDPLEYENLRVSFATQNYE

SEQ ID NO: 80 - protein, 472 aa, Fig cryptic virus
MEAGLIEIGNIPERHLRDEFIILVDQPAYDSVRRNAPQADMQEIDGWARSFYTVEGIMASIMQFSKPLIHEPTDPIWNDVKRETLMKIGSLFPQVQSLPFEGGFDHVPFESSSSAGYGYDGKKGEGNNFHRAKSIANAAVRKFSEDIDNQGYDYAVSHLIQQGTPDIAFTRTQLAKLPSIKVRIVFGEAFHNILIEGLSAAPLLEAFKRMDTFYFTGKDPTIYVPRILHKMSTNEGWFICLDWKAFDASVQLWEIDHAFNCIQQLLAFPTELSRLAFLFTRESFKQRKLADPNGILWMRKGGIPSGSYYTNIIGSVINYNRIEYVCKRLGLQKTSCYVQGDDSLIHITGDAKPDLTQLQMLGEQFGWTLNIPKCSLTQDSQLVTFLGRSQMHQLNIRERLKVLRLMCFPEYKVEDPKISTTRVKAIARDAGWSDPVYNKIYLQLKRLYGEVERLPPHLATFVDRFDFQDVNM

SEQ ID NO: 81 - protein, 580 aa, Flammulina velutipes browning virus
MSDTLIDSFSRLTLSVKNFVFLGFTETQNYPQKSDSAILSHRKVVLNAFEKYLNPIEYNHVANEYKRSETDLDSTKAAFFKGDIPDHEVPRDEHYNRAFSVIVSKFRPPEPIRPVHYADLRLYPWPLKPSAEAPFSNDKSLLALLALRNRQGFLPNAKPNFHNLFNWVFGFNRQCVHLIKKGKDNLGPNEYWPAHGFLYPINIHTKSAIIGIHDPNKVRTIFGVPKLTVMVEAMFFWPLFRYYRFEQQSPLLWGYETMLGGWYKLNHELHLNPFYQGSILSLDWSFFDGRALFSVINDLYSDKGVKSYFEFNNGYIPTVDYPDSSTHPQKLHNLWDWMLTALKFAPCALADGTIWQRTVRGIASGQFTTQFMDSIYNGLMILTILSRMGFVIDETLPIKLLGDDSVTRLAVSIPASMHESFLIEFQRLADYYFSHTINVKKSKISNTPHNVSVLSYANNNGLPVRSRTSLLCALLYPKSRRPTWEHLKARAIGVYYASCGIDRTVRLICKDIFDYLDSQGIQASSAGLQDLFDPNFKSGTIPLDVFPSVEQVTTNLRSFHHLDNSDKERYFPTSHFLDTK

SEQ ID NO: 82 - protein, 585 aa, Heterobasidion partitivirus 12
MQTLLSAVYSIRDAIFGKTLSEPHGLNVNFHFDGYVTDEIKTSIPPRVEMWYSDYQKFLNPIIRANFTGAEADKIINGYHHPVATIPFMVENLKKGDLSDHPVPHDEHYLRARKMAADAFRPPRPIRPVHFADLRFYNWNWHPNVEEPYYSNKRAQEYVQAAHTLGLTPDARMSFGNLRDYVFMDTRHYLHQIKRNEISNPKTLWPLMKIHVKPALTGTDEQKIRVIYGVSKRHVLPQCMFFWPLFRHYIENDTDPLLWGFETILGGMMKLNSLMHRLYFQTFVTVDWSGFDLRSLFSIQREIFDDWRTYFDFSNGYIPTVWYPNSTADPIQLERLWNWQRDACFNMPFVMPDKSVYRRLFRAIPSGLFVTQFLDSHYNYIMLLTILSAMGFDITIERIRILVQGDDSLKNLIFFIPANQHDNFKAEFQRLATYYFDHVARPEKTEIYNSPQGVTVLGYTNNNGFPTRDPIKLMAQLYHPRQVGERWKSVLMAKCAGFAYASAYNYPKVTATLKTVYYKLAAKGFSPAALRTQRDIVLFGEAKFQVPTDHFPTAEEVQRHLRVPYKRTEEDSESYFPMKHFLDFA

SEQ ID NO: 83 - protein, 581 aa, Heterobasidion partitivirus 13
MLPIISQVTDYVYRKFIKPAPTQFKNNYLFQNWLSPLTTPHRDATKYAEYQAYLEKHIRSNLLGSDAEYVIKRFHHPIATIDSVNETLQRGDLPDHPVPKDEHYYRALTETTKRFAPPQLIRPIHFADLRYYEWNWHPNVEEPYVSNSQLKTAVQDAYHAGLLEDGRMSFGNLKNHVFMDVRHFLHRIKRGQISDPHTLWPLINMHVKPALTETDTTKIRLVFGVSKRHVLPRAMFFWPLFRYYLDNRDKSPMLWGFETILGGMMLLNSEMLLSRLYYQTFVTVDWSGFDLRSLFSIIREDIFPAWRTYFDFNNGYMPTKFYKSSTADPDQLERLWNWTNEAVFKMPFRTMDGATFLRLFRGIPSGLFETQFLDSFYNMLMILTILDAMGFDISTIYIRVQGDDSLLLLTFFLPADQHAEFKAQFEALAAYYFDHVARQDKTDISNTSQNVAVLGYSNDNGYPSRDWRKLLAQLFHPRSQRPTLSLLKARCCGIQYASMYKYPQVTNVAKATFNQLDSEGVQPVKLAAQRDVILHSHKDFYVPTDHFPTLNEVTRYLRIPYTRTEADSETYYPMSHFLSQF

SEQ ID NO: 84 - protein, 585 aa, Heterobasidion RNA virus 3
MKIFSTLYSSFASFAKWTGLTDPHGFEHNFYFTGYADKKIKVSINPRFQEVYDDYQSYVGKFIDKHLPGHMAQKIKFGYHHPVASLPFMITNLKKGDLPDHPVPHDQHYTAARKAAADAFRPPRLVRPVHFADLRYYKWNWHPNVEEPYYSDPKLQQYVEHCYALGLIDDARLSFGNLKDFVFMDTRHYLHLIKNGSITDNNQLWPIMKIHVKPALTEPTETKIRVIYGVSKRHILAQAMFFWPLFRYYIEEHTSPLLWGNETFTGGMLKIHNLISVPRLYSQTYLTVDWSGFDLRSLFTIQREIFDDWRTYFDFTAYIPTRTYPDSKTDPIRMERLWNWQRDACFKMPFVLPDRTTYARLFRSIPSGLFVTQFLDSHYNLIMIYTILSAMGFDITNLMILVQGDDSLIHLKFFLPADQHDAFKAEFERLAKYYFDHIARPEKTHVTNSPNEVEVLGYTNNNGYPSRDMTKLVAQLYHPRNVDKTSWKSLLMAKVCGFAYASCYQDSQVIDLLRSIYNNLASKGFKPKSGRVMRDIILFGESEFEVPTDHFPTLNDVTKYFRRPYVRTQRDADSYFPSWHFNDVF

SEQ ID NO: 85 - protein, 479 aa, Pepper cryptic virus 1; identical to SEQ ID NO: 1

SEQ ID NO: 86 - protein, 478 aa, Pepper cryptic virus 2
MAIYRMLNGYVFTAFGNDLEKLDQRHIHHIRREEATTYRDEFALKELMDLSPILYEQFLEGWSRSYYEGSKHLQAIIQYGIPDPDPGLIDNDIYNKAGYVVLESLGSLPRVRAFDVLTELDSVHYEQSSSAGYDYHGPKGPIQGENHIRAITRAKATLWSAIKDEGEGIEHVIRSAVPDVGYTRTQLTDLYEKTKIRGVWGRAFHYILLEGTIANPLLDVFKRGGTFYHIGENPQYSVPDILSQVSECCKYLVAIDWSNFDATVARFEINMAFDLIKTLIMFPNIETELCFEICRQLFIHKKIAAPDGNIYWSRKGIPSGSYFTSIIGSIVNRLRVEYIFRKAYDVGPKMCYTQGDDSLIGVDFRVDPDRLSEIAAPLNWKLNPAKTDVSLYPEHVTFLGRTMYGGINQRDLKRCLRLLIFPEFPVPSGEISAYRAVSIAQDAGGTSEILNSIAKRLRRQYGVAEEHAVPKHFKLYIP

SEQ ID NO: 87 - protein, 477 aa, Persimmon cryptic virus
MALRSITGYEFHDFQSSLELLNQTHIHIVRRESGVTYHDEFALCELLVDNTRLYEQELEGWSRSYYTGEQHMKAILQYSLPNTPIQDIDVGCYQQAMTNVQERLSSLPIVRAFDVLTQLDQVSFESSSAAGYDYTGAKGPKNEGNHERAIRRAKAVLWSAIAQDGEGIEHVLRSSVPDVGYTRTQLTDLSEKTKVRGVWGRAFHYILPEGTSADPLLQAFKEGGTFYHIGQDPTVSVPYILSDTAGKCAWLYALDWSSFDATVSRFEIHAAFDLLKQRIEFPNFETEQCYEICRQLFIHKKIAAPDGKVYWAHKGIPSGSYYTSIIGSIINRLRIEYIWIKLRGHGPTICYTQGDDSLCGDDERIEPERIADIANPIGWLINPAKTATTRYPEYITFLGRTCYGGLNHRDLIRCLRLLIYPEYPVPSGAISAYRANSIAEDCGGTSSILNDIARRLTRKYGRVSHEEVPKELRVYRH

SEQ ID NO: 88 - protein, 479 aa, Rose cryptic virus 1
MEHRFRGIPRGLIELEEIPTRRLREECVIHIDAWSSQAIDAIVPLSLRNELDGWARSYYTLQAHVDSLMQYDRPKLQPPTNTAWNITTQYIRTEFARMKKVTALSYLQLDQVKWVRSSAAGYGYTGRKSDGDNYIRARKTAFTLAEKLNHNRDYGPLALEDSTPDVAFTRTQLCQIKVKRKIRNVWGEAFHYVLLEGLFADPLIQQFMRIKSFYFIGEDPLLAVPRLIEEILSEQDYIYMFDWSGFDASVQEWELRFAFGLLESILIFPSSVEHQVWQFIIELFIYRKIAAPNGKIYLKTLGIPSGSCFTNIIGSIVNYVRIQYMFFRLTREFVTAFTHGDDSLVGVPTTQYVQMENFKPICDENLWTINIAKSAISREAEGVSFLSRKVREMCHARDELICLRMLKFPEYIVETGAMSTLRAFSIHKDAGIHSRYLYQIYKFLLHRYGKADSLPLNQQNWDPIEYENLRVSYATQNYE

SEQ ID NO: 89 - protein, 479 aa, Rosa multiflora cryptic virus
MEHRFRGIPRGLIELEEIPTRRLREECVIHIDAWSSQAIDAIVPLSLRNELDGWARSYYTLQAHVDSLMQYDRPKLQPPTNTAWNITTQYIRTEFARMKKVTALSYLQLDQVKWVRSSAAGYGYTGRKSDGDNYIRARKTAFTLAEKLNHNRDYGPLALEDSTPDVAFTRTQLCQIKVKRKIRNVWGEAFHYVLLEGLFADPLIQQFMRIKSFYFIGEDPLLAVPRLIEEILSEQDYIYMFDWSGFDASVQEWELRFAFGLLESILIFPSSVEHQVWQFIIELFIYRKIAAPNGKIYLKTLGIPSGSCFTNIIGSIVNYVRIQYMFFRLTREFVTAFTHGDDSLVGVPTTQYVQMENFKPICDENLWTINIAKSAISREAEGVSFLSRKVREMCHARDELICLRMLKFPEYIVETGAMSTLRAFSIHKDAGINSRYLYQIYKFLLHRYGKADSLPLNQQNWDPIEYENLRVSYATQNYE

SEQ ID NO: 90 - protein, 573 aa, Raphanus sativus cryptic virus 1
MSNLEYLGLDHHWPAIPRFAKSPNDWYFACQQLVRSSICLYSSLLGSNDTDTVLNGYYRSHADEDTAEQFFMRYDVEPFDIIKDNYYSQAFDTVTEWFRPSAPIHPVHFTDVRWYPWKISTSAERPFTHDPLLKKKVQLSKQLGLLDNARMSFHNCYNDIFTYCRHYTHEVKDARPVTLHHIDLHVKPALVRSGEPPKIRTVFGVPKSLIFAEAMFFWPLFSDYFTNSETPLLWNYETLNGGWYRLNDEFYQQWQSFCTIFNLDWSEFDMRVYFSMLDDCRDAVKSYFCFCGNYCPTRTYPTCRTNPQRLQNLWNWIGTAYKDTPCTTTTGKVYRRRFAGMPSGIFCTQFWDSFYNCIMVVTTLEALGFRITDRYFLKVLGDDVIFGILKHIPISKWADFLQDFSTEARRRFNSKLNSKKCGASSGIHGAQVLSYINWNGYPKRDSNQLLAQLLHPKSLRDTYPRLMARAIGIYYASCGDPKIRPICNHIYSELKYAGFTPSSTGLHGLFDPNASIGFIELDHFPSENEVTCRLHRKSKRSAELQALYWPRDHFLEEAGSSRNCPLSFQVETI

SEQ ID NO: 91 - protein, 477 aa, Raphanus sativus cryptic virus 2
MDHEFRKIREGLIEIGTVSLRVQRDEFKVIIDEYAAEAVFKFVPSTMLSQLQGWARSVYSLDQHVDAILAYRRQKLPEPTDDVWNQTKQHTLQLFRRFPKITPISYKSFDEVKWISSSSAGYGYVGHKGDGDNYLKARRTAVTIAEKLDHDRNYAPEAINQSTPDVAFTRTQLSQVKVKTKVRNVWGEAFHYVLLEGLFADPLINFFSNEESFYFIGRNPLLSVPTLIEEIFKSKDYVYAFDWSGFDASVQEWEIRFAFQCLESQLIFPSNVEAQIWRFIVELFIYRKIAAPNGTLFLKTLGIPSGSCFTNMIGSVVNYVRIQYMFKKLTDDFVEAYTHGDDSLAAVSTAQYIPLEKFGPICEPFMWSINTLKSEVSREGRLTTFLSRSIRDKQNYRDEFVCLRMLVYPEYEVEDGSISALRAKSIYVDAGIHSQYLYHVFLYLKQKYGLANTLPHNLRTWDPTEHEALRASYSNIM

SEQ ID NO: 92 - protein, 496 aa, Rhizoctonia solani dsRNA virus 3
MVDALRKGDLPDHVIPKDEHYSKAFAQAAEMFRPPQLVRPVHFADLRMYKWNWHPNVEEPFYSDADLIRAVSMAAEAGLLPDARMSFGNLRNVVFIKARLFLHQIKRKQITNPATLWPMMKIHVKPALTKVDETKVRIIYGVSKLHVMAQAMFLWPLFNYYINSDDDPLLWGFETILGGMQKLHNIMSIPRLYFQTFVTVDWSGFDLRSVFSLQREVFDVWRTYFDFNNGYIPTKFYRTSVADPDHLEALWEWQREACFKMPFVMPDRTMYNRLFRCIPSGLFSTQFLDSHVNLVMILTILDAMHFDISKIKIYVQGDDSIVMLIFHIPADQHIKFKSDFEVLAKYYFDHVARPEKTDVYETPQGVEVLGYRNYNGYPERDWRKLLAQLLHPRGALSLETLAARCCGIAYASMYRNPEVINVCKDIYNYLTTKRNVVPGELRAQRDIILFGEHEFSIPTDHFPERDEVTRHLRIPYVRTDSDKNDYWPSGHFLSLY

SEQ ID NO: 93 - protein, 478 aa, Sinapis alba cryptic virus 1
MRPSITGYDYTNFTQDLLKSDRKHPHVVRRETATTYRDDFAFKEVISLDRLAYIQRLEGWSRSYYLPEKHLEALLQYATPNVPCTALNLNVYRQAIQVVENGLRSLQPVRAFDVLTELNQISYKQSSAAGYDYIGAKGPIDGENHKRAISRAKAVLWSVVKEDGEGIDHAIETSVPDVGYTRTQLADLTEKTKVRQVWGRAFHYILLEGLVAQPFIQSIMEGPSFIHTGRDPTLSVPQSLAKVSSQCKYIYSLDWKSFDATVNRFEINTSFDIIKSKVIFPNYETEQAFEITRQLFLHKKVAAPDGYIYEAHKGIPSGSYYTSMVGSIVNRLRIEYIWRIATGHGPIHCETLGDDSLCGDDIFVPATQLADIANRIGWYFNADKTEYSTIPEGVTFLGRTSTGNLNSRDLTKCLRLLVYPEYPVTSGRISAYRARSIADDSGGLSDLLNQVAIRLERSYGIASEEEIPAYFKRYVPFM

SEQ ID NO: 94 - protein, 542 aa, Aspergillus fumigatus partitivirus 1
MEDYTQDPTQHYVLAKGSHLIDALQLRPARSGKSSTTSYDVLPSNFESDTLREIARYGGYSTYSSASNTDPWVRESLKIFDRDQYEAIRGFTRRPQGTPGMYESLKKFSTEERSTFWSLSPQQRTSMRRAIGKAKRAFKLPYKREPLDWHEVGQFLRRDTSAGATFMGQKKGDVMEQIYHEARWLGHRMKQDGIGPFQPHKMRFPPCLAGQRGGMSERDDPKTRLVWIYPAEMLVVEGFYAPLMYRDFMNDPNTPMLNGKSAQRLYTEWTCNLREGETLYGIDFSAFDARVPAWLIKAAFAILRQNVDFSTFRGKPVNKRDAQKWRNVWDAMVWYFINTPILMPDGRMFRKFRGVPSGSWWTQMIYSVVNYIMIEYLADCQRVEIRNLRVLGDDSAFRSGDQFDLDVAKGDAEPTQMLVNTDKSGKSKDPADFKLLGTTYRCGRVHRPTDEWFKLALYPESSVFSLGLSFTRLIGLWLGGAMHDATFSRFIEFFQQCYPCPEEGWFSKDQRRWLEIVYSGKAPRGWTTKKSLFWRSIFYAYG

SEQ ID NO: 95 - protein, 539 aa, Aspergillus ochraceous virus FA0611
MDDSHLDPTQLENVIEESLLLDDSTLTPSAKVRGASYNVIPPQFSSPGLSEIARYGGYGTYSGQSNTDPWVRVALKNFDRNVYDDVYGFTRKPEGTPGMYKSLFKFAEGRSDFRSLNRAQRKAMQAAISKTKKRFKLPYKSDPLDWHAIGQFLRRDTAAGATFMGCKKGEVMEDIYHEARWLAHRMKQDGRQRFNPKQMRFPPCLAGQRGGMSEASDPKTRLVWIYPAEMLVIEGQYAPTMYHKFMADPHTPMLNGRSSTRLYTDWINDAKEGDKLYGLDFSSFDSKVPSWLIRVAFNILRQNINFETWNGQPVSKRDRQKWRNVWDAMVYYFINTPILMPDGRMFRKYRGVPSGSWWTQMVDSVVNDILVQYICLCQEIEPRDLRVLGDDSAFRSCAELVLVQAERDAKDVNMVLHPEKCDVKTDPTKMKLLGTTYRNGRAHRDTDEWFKLVLYPESSVRTIEVSFSRLIGLWIGGAMFDSAFCRFMEYYQTCFQCPEDGWFSKEQRRWLEVVYGNRAPRGWSAKKSLFWRSIFYAYA

SEQ ID NO: 96 - protein, 540 aa, Botryotinia fuckeliana partitivirus
MEEFTQEPTQHYVLAKGSHLIDALHLRPDTGKGSTTSEDVLSSDYRSPNLAEIARYGGYSTYSSNSNTDPYVRETLKLFSRDTYEDIRGFTRRPEGTPGMYKALEKFSGEKNSFNDLSATQKSSMRRAIGKAKKAFKLPYKREPLDWHEVGQFLRRDTSAGSTFMGQKKGDVMEEIYHEARWLGHRMKQDGKGRFNPTKMRFPPCLAGQRGGMSERDDPKTRLVWIYPAEMLTVEGFYAPLMYRDFMNDPNSPMLNGKSAQRLYTEWCCKLREGETLYGIDFSSFDTKVPAWLIRIAFDILRQNIEFSTFQGKPVSKKDAQKWRNVWDGMVWYFINTPILMPDGRMFRKFRGVPSGSWWTQMIDSVVNHILIDYLADCQDVEIRNLKVLGDDSAFRSSDEFQLETAKLDCKPTGMVIKPEKCEKTADPADFKLLGTKYRSGHVHRDTDEWFKLALYPESSVFTLDVAFTRLIGLWLGGAMWDKRFCEYMDFFQSSYPCPEEGWFSKDQKRWLEVIYSGKAPRGWTTKKSLFWRSIFYAYG

SEQ ID NO: 97 - protein, 539 aa, Colletotrichum truncatum partitivirus 1
MEDFTQDPTQHYVLAKGSHLIDALHLRPAKPGSTTSEDVLSSDFESPNLREIAKYGGYSTYSSNSNTDPWIRETLKIHDRETYEQIWGYTRRPQGTPGMYTALGKFAGEKNVFGDLSSSQQSSMRRAIGKAKKAFKLPYKREPLDWHEVGQFLRRDTSAGSTFMGQKKGDVMEEIYHEARWLGHRMKQDGKRSFDPTRMRFPPCLAGQRGGMSERDDPKTRLVWIYPAEMLVVEGFYAPLMYRDFMNDRNSPMLNGKSAQRLYTEWCCNLREGETLYGIDFSAFDTKVPAWLIRAAFSILRQNVNFETFQGKPVEKEEAQKWRNVWDAMVWYFINTPILMPDGRMFRKFRGVPSGSWWTQMIDSVVNYILIEYLADCQKVEIRNLRVLGDDSAFRSGDQFSLESAKIDCIPTGMIIKPEKCERTKDPSDFKLLGTKYHDLHPFRDTEEWFKLALYPESSVHTLDISFTRLIGLWLGGAMWDRKFCEFMDFFQTSYPCPEEGWFSKDQKRWLEVIYSGKAPRGWTTKKSLFWRSIFYTYG

SEQ ID NO: 98 - protein, 539 aa, Discula destructiva virus 1
MEEFTQDPTLHNVQAEESHSIDTLHLRDAKRGSSTSEDVLHKGYADPCLREVAKYGGYSTYSSNSNTDPWIRETLKIHDRETYEDIWGKTRRPEGTPGMYKALGRFGGEKCDFDNLSDPQKSSMRRAIAKAKKAFKLPYKREPLDWHEVGGFLRRDTSAGSTFMGTKKGDVMEEIYHEARWLGHVMKQDGRKGFDPTKMRFPPCLAGQRGGMSDRTDPKTRLVWIYPAEMLVVEGFYAPLMYHDYMNDPKSPMLNGKSAQRLYTEWCCGLRDGETLYGIDFSAFDSKVPAWLIRVAFDIVKQNINFETFEGKPVDKHDAQKWSNVWEAMVWYFINTPILMPDGRMFRKYRGVPSGSWWTQIIDSVVNNILIDYLADCQQLEIRNLKVLGDDSAFRSTDQFDLEVAKDDCVPTGMVIKPEKCERTEDPNDFKLLGTKYRDGRVYRSTDEWFTLALYPESSVLTLDVSFTRLVGLWLGGAMWDKQFCAFMDYYQTSYPVPEEGWFSKDQKRWLEVVYSGKAPRGWTTKRSLFWRSIFYAFG

SEQ ID NO: 99 - protein, 539 aa, Discula destructiva virus 2
MEGFTQEPTNTTVLAEELHSVDTLHLRPGKTRSTTSEDVLPNNYEDPCLREIAKYGGYSTYSSNSNTDPWIRETLKLHDRQIYEDIWGKTRRPEGTPGMYKALGRFGGERCGFDDLSSQQKSSMRRAIAKAKKAFKLPYKREPLDWHEVGQFLRRDTSAGSTFMGSKKGDVMEEIYSEARWLGHRMKQDGRSRFDPTKMRFPPCLAGQRGGMSDRDDPKTRLIWIYPAEMLCVEGFYAPLMYRDYMSDPNSPMLNGKSSQRLYTEWCCNLREGETLYGIDFSAFDSKVPAWLIRTAFDIVKQNINFETFEGKPVNKVDAQKWKNVWDAMVWYFINTPILMPDGRMFRKYRGVPSGSWWTQIIDSVVNNILIDYLADCQSVKIPKPEVLGDDSAFRSNDQFDLEVAKDDCVPTGMVIRPEKCEKTEDPAEFKLLGTKYRSGRVHRSTDEWFSLALYPESSVLSLDVSFTRLVGLWLGGAMWDKQFCEFMDYYQTSYPVPEEGWFSKDQKRWLEIIYSGKAPRGWTTKKSLFWRSIFYAYG

SEQ ID NO: 100 - protein, 539 aa, Gremmeniella abietina RNA virus MS1
MSEVDNTDPTLQDVAFVKSSGLDTTHLRSSEKTAGASYSVISSNFNSPGLTEIARYGGYSVYSGNQNTDPWVRATLKNFSRETYEQIYGFTRQPEGVKGMYSSLLKFSDGKCKFDRLNRVQRKAMIGAIAKAKKAFRLPYKSEPLDWHQVGAHFRRDTAAGVSFMGKKKGEVMEEIYHEARWLGHRLKQNGKARFDPRQMRFPPALAGQRGGMSKRDAPKTRLVWVYPAEMLVVEGQYAPVMYRAFMDQPDTPMLTGASSQRLYTEWLVGRREGETLHGLDFSSFDTKVPSWLIRVAFDILRQNIEWETWQGEKVSKRDRQKWRNVWDGMVWYFINTPILMPDGRMFRKRRGVPSGSWWTQMVDSVVNYILVEYLTECQGVEARGLRVLGDDSAFRSPVEFSLEQAQSDCEPTGMILKPEKCEKTEDPSDFKLLGTTYRGCHPHRDTNEWFKLALYPESRVGNLEVSLSRLVGLWIGGAMWDKEFCSYMDYFQSSYPCPTEGWFSKDQRRWLSIVYGGKAPRGWGDKKSLFWRSIFYTF

SEQ ID NO: 101 - protein, 539 aa, Magnaporthe oryzae partitivirus 1
MEKTSPDLPFTKDVLTSADWDAVHRSLSYGNPGLTKIPADKWCYKYNVEQTRMNTDPFVRKAMKLWDETEYKQLYGYTKKASLEHGLNGLNKYGRPQRQKSHMPSEFKGSYHRALQEATRVFTPHEPLHRLSVPDVWDNMNLDSAAGFTFPGKKKSEVVEEAFDTASYMAHFISAGKHIYVPPAKLALRGHLSELEEIKTRPVWVFPFEVTILEGKWAIPYYRFLEEEVPSVHFGEGAMQRLAKILDSDIASHAEYAELTMDWSGFDTGVPNWMTDDALDILFGAFDETAVQHQDDLVVGGEYMAYKNEAVKDFLKTYFKKTKILLPDGSVYKKNHGIPSGSFFTQAIGSIINYIAVRTLDFYFGWNGRRFKVLGDDSSYLIPNGLGKVSVDAVSKAAWAAFGFTLKREKLRIATKQHERKFLGYQVSAYRYERPSDDWLKMALYPERDCEFLEQSASRVFAFYLLGGCNDATYCDFFHDYLNRYPVVYGSELPLTKGLKRLFKFVLRLNVERLVFLDLPNFDPLKGPFALSLGDKPFG

SEQ ID NO: 102 - protein, 519 aa, Mycovirus FusoV
MVELFVMVDPTTKRRRIQSTLGPFLSVPGLQEIARYGGYATYRAVRNTDPWIRQSLKLFDPDLYGNIYGFTRRPAGPEGMYKSLMKFGESMPRFTDMSTVQRSAMKTAITAARKRFKTPVKFEPLEWSEVGQHMRRDTSAGVSFPGKKKGDVMERIYAEGRWLGHRMKQGGKGRFDPRKVRMPPCLATQRGHLSPRDDPKTRLAWIYPSEMLMVEGLYAPTMYKAFEAMPDSPLLLGKGSHRLFSEWVSAATPGMRLYGLDFSSFDTKVPAWLIHTAFDILHDNIDWLHWRGKPTTKRSRQKWKNVWDGMVYYFINTPILMPDGRMFRKRRGVPSGSWWTQLVDSVVNWILVKYLSLCQGVNAKNLRVLGDDSAFMAAETMDLSVAAEDAAAVGMDLSDEKSISVEDATELKLLGVRYRDGHAFRETEEWFKLALYPEGDVPDIATSLTRLVGLWIGGAMWDTKFSRFMEYFQGCYPCPSEGWFSKDQRRWMEIVHGGRAPRGWTKNKNLFWRSIFYTL

SEQ ID NO: 103 - protein, 538 aa, Penicillium aurantiogriseum partitivirus 1
MAFTQEPTQHYVLAKGSHLIDSLHLRPAKAGSATSEDVLPTGYNSPNLREIAKFGGYSTYSSASNTDPWVRETLKLFSRERYEEIYGFTRRPEGTPGMYKSLAKFAGEKSHFRDLTVSQQKAMRRSIAKAKKAFKLPYKREPLDWHEVGQFLRRDTSAGSTFMGQKKGDVMEDIYHEARWLGHRMKQDGESSFNPTKMRFPPCLAGQRGGMSERDDPKTRLVWIYPAEMLVIEGFYAPLMYRDFMNDPNSPMLNGKSAPRLYAEWCCGLREGETLYGLDFSAFDTKVPTWLIYTAFDILRQNIEWSTFQGKPVSKQDAQKWRNVWDGMVWYFVNTPILMPDGRMFRKYRGVPSGSWWTQMIDSVVNYILIDYLAECQEVEIRNLRVLGDDSAFRSTDQFSLEQAKVDCEPTHMLLKPEKCEKTKDPCEFKLLGTTYRDGRVHRPTEEWFKLVIYPESSVHTLDISFTRLIGLWLGGAMWDKEFCRYMDFFQSSYPCPEEGWFSKDQKRWLEVIYSGKAPRGWTTKRSLFWRSIFYAYG

SEQ ID NO: 104 - protein, 539 aa, Pseudogymnoascus destructans partitivirus-pa; identical to SEQ ID NO: 2

SEQ ID NO: 105 - protein, 538 aa, Penicillium stoloniferum virus F
METTTPDLPFDLHTREAYDYATFHRTLLHKPGLSRIKEDRWVYKYNVEQTRMNTDPFVRKSMKLWDEHAYHDMYGFTKKARLSNGLDAFQGFAKPQKQRSSMSPEMASCYEKALEEARHVFTPHERLTRLSVPNVCDSTNLDSAAGFSFPGKKKSEVVEEAFDVASYIAHFVASDRKVFIPPAKLALRGHLSEIDELKTRAVWVFPFEISILEGKWALPYYKFLEQNVPEVHFGEGAMQRLAKTLMTDVASHSECTEVTLDWSGFDTSVSNWLIDDAFDIMFDSFDETQVEHDGNFVLGGDHMAKKNEKVKKFLKTYFKKTKIMLPDGSLYKKFHGIPSGSFFTQIIGSIVNYLAVKTLDNYFSWNARRFRVLGDDSSFLIPFGRSKVDGVEISEKAWETFGFTLKLKKLRIANKQQDRKFLGYQCNAFRYERSTTEWLSMVLYPERDVEFLEQSASRVFAFYLLGGCNDVTYCEFFHDYLGRYPYIYGKELPLTRGLKRLFKFVFRLTIDKLAFPDLSRFDPLKVPFSLSLGDKPFW

SEQ ID NO: 106 - protein, 539 aa, Ustilaginoidea virens partitivirus
MEDFTQDPTHHYVLGKGSRLIDALHLRPAKEGQANSEEIVPSNFKSDTLREIAKYGGYSTYSSNSNTDPHVREALKLFSRDIYEDIRGFTRRPQGTPGMYGALAKFSGERNAFSDLSASQQASMRRAFSKAKRAFKLPYKREPLDWHEVGQFLRRDTSAGVTFMGAKKGDVMEEIYHEARWLGHRMKQDGRDSFDPTRIRFPPCLAGQRGGMSEIDDPKTRLVWIYPAEMLVVEGFYAPLMYRDFMSDPNSPMLNGKSAQRLYTEWCCNLREGETLYGLDFSAFDTKVPAWLIRVAFDILRQNIEFSTFQGKPVNKEDAQKWRNVWDAMVWYFINTPILMPDGRMFRKFRGVPSGSWWIQMIDSVVNHILIDYLADCQDVEIRNLRVLGDDSAFRSSNQFDLEVAKQDCVPTGMVIKPEKCERSEDPSDFKLLGTKYRGGHVFRPTEEWFKLVLYPESSVLSLDMSFTRLIGLWIGGAMWDRKFCEFMDFYQSAYPVPEEGWFSKDQKRWLEVVFSGRAPRGWTTKKSLFWRSIFYAFG

SEQ ID NO: 107 - protein, 539 aa, Verticillium albo-atrum partitivirus-1
MSDDIEIFDLLASPGEQPGSDETFASYGTPSSRTARRHVQAGEYFIGTGLEEIARYGGYSSHRASGNTDPWVRETLKLYDPERYESIYGFTRTGEGLLGAYKSLFKFDGPVARGTRLSTRQRSAMKKAIADARTAFKLPVKHEPLDWHEVGQFVNQSTSAGVSFPGKKKSEVMEEIYTAARWLGHRMKEGGKESFNPTKVRFPPALAGTRGHMSPKDDPKTRLVWVYPAEMLVVEGLWAPVMYRQYQSLSDGPLLLGKSAQRIYTEWCVNKKQGEVLHGLDFSGFDNGVPPWIIHVAFDILHANVDWLNWRGKPTSKRSRQKWRNVWDGMKWYFINTPILMPDGRMFRKHRGVPSGSWFTQLVDSVVNYILVKYAFNCQELKIHGLKVLGDDSAARSPLKLDLVQAAVDFRPVQMRLNLDKCEITEDATEFKLLGTRYQDGHSTRPDEDWFKMALYPENPPPDIAVSMTRLVGLWLGGAMWSADFCKFFEYFQSSYPCPSEGHFSKDQRRWLEIVFGGSAPRGWTYKESLFWRSIFYVF

SEQ ID NO: 108 - protein, 539 aa, Verticillium dahliae partitivirus 1
MEDFTQDPTTHNIVAEGSHLIDALHLRPPKLRSTTSEDVVPSKFRSPNPIDHDAMRGYSTYSSNSNTDPYVRETLKLFSRDTYEDIRGFTRRPQGTPGMYTALKKFSGERNTFGDLSPSQQSSMRRAIGKAKKAFKLPYKREPLDWHEVGQFLRRDTAAGATFMGQKKGDVMEEIYHEARWLGHRMKQDGRAGFDPTQMRFPPCLAGQRGGMSEIDDPKTRLVWIYPAEMLVVEGFYAPLMYRDFMSDPNSPMLNGKSAQRLYTEWCCKLRDGETLYGIDFSAFDTKVPAWLIRVAFDILRQNVNFETFGGKPVEKRDAQKWRNVWDAMVWYFINTPILMPDGRMFRKFRGVPSGSWWTQMIDSVVNHILIDYLADCQRVEIRNLRVLGDDSAFCSGGQFDLELAKGDCENTGMVIKPEKCERTKDPGEFKLLGTTYRGGHVFRDTEEWFKLALYPESSVLTLDISFTRLIGLWLGGAMWDKKFCEYMDFFQSSYPCPEEGWFSKDQKRWLEIIYSGKAARGWTSKKSLFWRSIFYAYG

SEQ ID NO: 109 - protein, 755 aa, Fusarium poae partitivirus 2
MHSLFTIVNLLLLARFQLRRKRKETTKTLIYPIRNILFEWELRRSTPREPGIPLRYFDYSKSLLSYIDLQTCHKINIHLDDHTDALMERYASRDEPFAVYQHISDEDLPPERTPAPGIRHAQCRYHEIPSGTLNLDEKQHVLTDDPDFIETPEFRSGILYDETIDLSGSPPIPEIAQIIHDWFPHFEPFLAEYCRPPSFGPQAFRDFNRPTPHPPPPPHERHEAIMDIVRAKFNLKPYRPMHYVDALAAETPLNTSASYYSKFNPTSRVFARYSAPSRYKDKPTSKGYNFNVVMNEFRTEYHHIKYDGVPFPADLHDPEANASILNTWFAKHPSQLFIRTQISKRDPNDPKKIRPVYSVDDRFLHIEKTLVVPALAQLRNPQCCVAHGLETFRGSMSLLDRTALVYTSYISLDWSQFDQRLPYYVIIAFFLDFLPSLLIISHGYFPSRGYESTPQDIHAFASKIFNVVLFLTTWYLNMTFVSFDGFAYIREHGGVPSGLLNTQFLDSFGNMYIIVDCLLEFGFTPAECLDMLYCVLGDDNLIFARQNFDRICDFMIFLTKYADTRHGMVVSILKSVYTKLRSKISFLSYENTYGMPTRPIGKLVAQLSMPERPIPDNRKWIHAARALGLAYANCGQDAHFHLLCHMVYEKFRPDTPVPSLHIEKVFKKWKYQLPEFDIESVTYTFPDFPTLFSIRQLVSDYHGFFSETDKWNTDMFEAPPSDNLHDYVTLKEYMNSNVHMSHTVNEFMHGKRSFL

SEQ ID NO: 110 - protein, 722 aa, Heterobasidion partitivirus 2
MSTNPPEVLLPLPEVDPNANNVRYAKLLDQYRANPSNSNQKLLLEYAEQHGFNYFIPTDVPLPDDRKSAPGILSLDGFRFHTVAAFHRYQKMSRYGYNALGFIFKLILTLFNPFLWILTDYCRPSGSADAVFENFNQEVSPVEHVNPSRLAQIMPLIHHFFAIKPFCPIAFPDLRFYKWSLVTSADYHAHHSKDQQDESAHYWKHLKDSDLLQDRFDYSDRPRSKGYFFNTVLLSTRTIVHNIKYHCLPFQRHKRDTDSSVLQKLSFWFMKYPTVMYVRSQISKLSKLKVRPVYNAPFLFILIEAMLTLALMAQCRLPDSCLMWGFETVRGGMQELNRISYNYDTFIMIDWSRYDQLLPFAIIYHFWCTFLPQLIRVDLGYMPTEQYTATQHKHAFTEKHNDQKESNPEYATFASRLKTHAPHIVMFSFIIFNLLAFIWLWYVKMVFVTPDGFGYVRLLAGVPSGIFMTQICDSFCNAFLLIDAMLEFGFTPDDIKLIRMFIQGDDNVIFYLGDFTRIFAFYEWLPEYCQQRWHMTISVDKSSITRLRNKIEVLGYTNLNGMPHRDCAKLIATLAYPERYVQDKTKYIVFMSRAIGIAYANAGHDRQVHDLCQRAYLQARKDSGLSYDELKNIKIEYQKLGFYEIFSVNIEELREHLIQDVSEFPNFHDIRDNLRHWHGPHTVYPMWPRHFDDDLSSIKSPHSLTTLYDVMQSGGLTFDYNF

SEQ ID NO: 111 - protein, 529 aa, Ustilaginoidea virens partitivirus 2
MNSFENFGSFKLSADELAATAVPPTPWNNVFRYITDAKRFPGYKRGILRQTQLYDPYVNAALKSFSPELHDSIKGYTRAPGDEWDVYERLTRYDKSPLAPVDNPRFKACYDAALSDVMKEFKLRDPVVPHWILDVDLVKNTSSGFPHFTRKGDILDQIRQEGRSHFHLLKRLPLWRVPLLPCTPATRGGLADITEPKTRLVWMYPAAMTAVEAVFAQPLIDGLFSEKSEYLITGVDTKHRIQRYLSLLSEDTGRLGVGLDFKSFDTLRCNWLIRDAFDVLKQNVYFSGYYDDTNGLQTFGPGKTERLEHAWSNIVEYFIHTPILLPNGRCVNKHTGVPSGSHFTNLIDSIICRILIKTFSLYCSIPISNLRTNGDDSAFHVYEDYASDIILRAAGFFKEFFGMTINTDKSCVAGSPSEMHVSGTRWTGLRPTRSTQEWMMLAAYGETYSRIPFDSFQRLLGLGLSGGFGDSTFTRFFDYFQTGYDCRHGPNLLNWKKLRFLQQIFSIEELPLVYKQGAKTTLRLRLLVT

SEQ ID NO: 112 - DNA, 1450 bp, artificial sequence
  codon optimized RdRp coding sequence
ggtctcaaggtatggttcgtggcaccctggttggctatgattacacccaatttcaaggcgatctggttaaaagcacccaccgtcacccgcatgttgttcaccgtgagatcgcgaccacctacgtggaccagtacgcgtatgagcacatcgaaaccttcagcagcctgtatccggagctgattctgaagggttggagccgtagctactatctgccggaaaaacacctggcggcggtgctgaactacagcatgccgaacgttccggcgagccagctgagccaaagcctgtatcgtcaggcgatcgagagcgcgaagaacggcttcattagcctgccgcgtgtgaaagcgtttgacgttctgaccgaaatggatcaagttccgtttaagagcagcagcagcgcgggttacaactataccggtcgtaaaggcctgatcggtgatgaaaaccacagccgtgcgatcagcattgcgaaggcggtgctgtggagcgcgattaaagacgatggcgagggcatcgaacacgttattcgtaccagcgtgccggacgttggctacacccgtacccagctgaccgatctgctggagaagaccaaagtgcgtcaagtttggggccgtgcgttccactacatcctgctggaaggtctggtggcgtatccgttcattcagaccgttatgagccacaagacctttatccacgcgggtcaagacccgctgattagcgtgccgcgtctgctgagcgatgttgcgctgaactgcaaatggatctacagcctggactggagccagttcgatgcgaccgtgagccgtttcgagattcacgcggcgtttgacatcattaagagctacgttgattttccgaactacgaaaccgaacaggcgttcgagatcacccgtcaactgtttattcacaagaaagtggcggttccggacggctacatctatgaaagccacaaaggcattccgagcggtagctactataccagcctggtgggcagcatcattaactacctgcgtatcaactatctgtggcgtctgctgaccggtcacccgccgcagcaatgccacaccctgggtgacgatagcctggtgggtgacaacagctacgttaacccgcaggcgatcgaggaagcggcgaacaagctgggctggcacttcaacccggataaaacccaatacagcaccgtgccggaggaaatcacctttctgggtcgtacctatgttggtggcctgaacaagcgtgatctgaccaaatgcattcgtctgctggtgtacccggagtatccggttgaaagcggtcgtatcagcgcgtaccgtgcgaagagcattgcgcaagacgcgggtggcctgagcgaagttctgaaccgtatcgcggataaactgcgtcgtatttatggcaccgcgagcgaagaagaagttccgatttatttcaagcgttatgtttttggcgtgta

SEQ ID NO: 113 - protein, 663 aa, Ceratocystis polonica partitivirus
MPSFSANPTYQTLIDDIDFDVPIAHPFSVINTDLQPVDDEETVHDLGSKDFEFYKVVSDNLPPTRAPSIGIESLPNIRYHNHSNDHRYRDQPPTGPPPMRGVRKIINDSFPQYLPYLKEWCRPKTSSDAIFEDFNQPQIPSIPLSYNRKQRILNLVNHFMGVKPYDIVHFCDTRFYPWDLSKKADYFHNHSNARKRHAQTSHASTATGPTKKSWFINAHLFHDRSTVHNIKLYGLPFKPHSYEARNKILLELWFKKIPTELLVRSHISNPKKLKVRPVNNAPMIFLRIECMLFYPLLAQLRKQQCSIMYGLETIRGGMMEIESLATRFSNFMMIDWSKFDQTVPFTLVDMFYQDWIPTLILVDSGYAKIHNYNDHVHSFAAQARKLGVHGDSNLNEAPPEAAVFANKVENLLKFINTWFKEMVYITPDGFAYSRTFAGVPSGILCTQLIDSFVNLVVLIDSLFEFGFHESDIKSALILLMGDDNVVFAPDKLSQLHSFFKFLPDYAKKRWNMKVNVDKSIFTTLRRKIEILGYTNNYGMPVRSLSKLIGQLAYPERHVNDSDMCMRAIGFAWCAAASDSTFHDFCRKVFIYYYARVNVPIKDLVQSNASALPGMFFAYRDVHQHIKLDHFPSIEEVRQVLSKHHGYLTEEPLWKYDFFLHPKP

(19) United States
(12) Patent Application Publication
(10) Pub. No.: US 2020/0157513 A1
(43) Pub. Date: May 21, 2020

(54) COMPOSITIONS AND METHODS COMPRISING VIRAL REVERSE TRANSCRIPTASE

(71) Applicant: The Penn State Research Foundation, University Park, PA (US)

(72) Inventors: Marilyn ROOSSINCK, Bellefonte, PA (US); Mahtab PEYAMBARI, State College, PA (US)

(21) Appl. No.: 16/604,249

(22) PCT Filed: Apr. 10, 2018
(86) PCT No.: PCT/US2018/026913

§ 371 Date: Oct. 10, 2019

Related U.S. Application Data
(60) Provisional application No. 62/483,651, filed on Apr. 10, 2017.

(51) Int. Cl. C12N 9/12; C12N 15/70; C12N 15/10

(52) U.S. Cl. CPC C12N 9/127; C12N 15/1096; C12N 15/70; C12Y 207/07048

(57) ABSTRACT

Compositions and methods for making cDNA from RNA templates, including double-stranded RNA (dsRNA) and single-stranded RNA (ssRNA), are provided. The composition and methods include a recombinant or purified or modified or recombinant RNA dependent RNA polymerases (RdRp's). The RdRp has an amino acid sequence that shares identity to a contiguous segment of the amino acid sequence of one or more Partitiviridae virus RdRps, and includes a reverse transcriptase (RT) domain. Kits including dNTPs for use in generating cDNAs are included. The RdRps can function efficiently at lower temperatures than previously available RT enzymes. Methods of making cDNAs are provided using an RdRp are also provided. RdRps can also be used to identify candidate reverse transcriptase inhibitors.

### CROSS REFERENCE TO RELATED APPLICATIONS

This application claims priority to U.S. provisional patent application No. 62/483,651, filed Apr. 10, 2017, the disclosure of which is incorporated herein by reference.

### STATEMENT REGARDING FEDERALLY SPONSORED RESEARCH

This invention was made with government support under Hatch Act Project No. PEN04480, awarded by the United States Department of Agriculture/NIFA. The Government has certain rights in the invention.

### BACKGROUND OF THE DISCLOSURE

The enzyme Reverse Transcriptase (RT) was discovered in two different laboratories in 1970 in the virus particles of RNA tumor viruses. RT copies RNA into DNA, a reaction thought to be impossible prior to this discovery. RT is found in RNA retroviruses, some DNA viruses (called pararetroviruses), and embedded in most genomes associated with retrotransposons and some types of introns. For the past forty-five years virus-derived RT has been used extensively for many molecular biology applications, including cloning, RT-polymerase chain reaction (RT-PCR), diagnostics, RNA sequencing, and the expression of many important pharmaceuticals that were first isolated as messenger RNA. RT is sold by many biotechnology companies, and is a standard component in the toolbox for molecular biology.

One RNA substrate for RT that has been proven to be quite difficult is double-stranded (ds) RNA. RT is, in fact, a single-stranded specific enzyme, and to use it for dsRNA requires several distinct approaches to make the dsRNA into ssRNA. Several methods are available for this process; the simplest method is boiling, but many protocols call for chemicals to keep the RNA single-stranded, and without the use of very toxic chemicals such as methyl mercury, the RNA very quickly reanneals to its dsRNA form. Some protocols use much higher than normal temperatures for the RT reaction, and while this helps keep the dsRNA melted, it also dramatically reduces the fidelity of the RT enzyme, introducing mutations that can be mistaken for single nucleotide polymorphisms (SNPs) with biological significance. All this makes the reaction very inefficient, and in many cases it fails completely.

The analysis of dsRNA as a hallmark of RNA virus infection has gained importance in recent years with the rise of virus discovery work. Virus discovery has revealed the amazing diversity and abundance of viruses in all environments, and the role of viruses in the ecology of life on our planet is slowing being clarified. The significance of the virome in human and animal health also is becoming increasingly apparent, and viruses can stimulate the immune system to counteract infection by pathogens, or act as early surveillance against incoming bacterial pathogens, as well as create risk for a variety of cancers. Other important biological roles for dsRNA are in RNAi, or gene silencing, and in the CRISPR adaptive immune system of bacteria and archaea that has gained widespread use for genetic manipulation of genomes from fungi to humans.

One additional problem with all commercially available RT enzymes is a relatively low fidelity. This has been particularly problematic for studies in RNA virus populations, and transcriptome SNPs. Biotechnology companies have developed a number of mutants of the RT derived from Avian myoblastosis virus that have increased fidelity (e.g. Superscript III, INVITROGEN®/THERMO-FISHER®), but they have not overcome this problem. Thus, there is an ongoing and unmet need for new technology for use in, for example, for siRNA and CRISPR work, as well as for RNAseq, and discovery and diagnostics of RNA viruses. The present disclosure is pertinent to these needs.

### SUMMARY

Provided are improved compositions and methods for making cDNA from RNA templates, including double stranded RNA (dsRNA) and single stranded RNA (ssRNA). In certain embodiments, the disclosure provides a recombinant or purified or modified recombinant RNA dependent RNA polymerase (RdRp), wherein the RdRp has an amino acid sequence that is at least 90% identical to a contiguous segment of the amino acid sequence of a Partitiviridae virus RdRp described herein, and wherein the contiguous segment comprises a reverse transcriptase (RT) domain.

In certain embodiments, the disclosure provides a purified or recombinant or modified RdRp that has RT activity for use in producing cDNA from RNA. In embodiments, the RdRp is modified, such as by having one or more amino acids changed relative to a native sequence, or one or more amino acids deleted, or added. In an embodiment, an RdRp of this disclosure is modified by including a tag, such as a purification tag, including but not necessarily limited to a histidine tag (His-tag). In embodiments, such as in methods and kits of this disclosure, the RdRp is present in or provided with one or more buffers that comprise deoxyribonucleotide triphosphates (dNTPs). Separate buffers or a single buffer can be provided such that the dNTPs used in the method and/or provided with a kit comprise all of deoxyadenosine triphosphate (dATP), deoxycytidine triphosphate (dCTP), deoxyguanosine triphosphate (dGTP), and deoxythymidine triphosphate (dTTP). In embodiments, the method and/or kit is free of added ribose-based NTPs, such as Uridine-5′-triphosphate (UTP), since the disclosure is directed to synthesis of DNA, rather than the ordinary function of an RdRp to synthesize RNA.

All intermediates formed during reactions used to generate cDNA are included within the scope of this disclosure. In an embodiment, the disclosure thus provides an isolated or recombinant or modified RdRp in a complex with an RNA template, wherein the complex is in an in vitro reaction. In an embodiment, the complex of the RdRp and the RNA template further comprises a segment of a cDNA that is complementary to one strand of the RNA template, i.e., the cDNA that is being elongated by the RdRp. In embodiments, cDNA synthesis is performed at a temperature of less than 50° C., or less than 40° C., or less than 30° C., or not more than 25° C. In embodiments, cDNA synthesis is performed at a temperature of from 10-25° C., inclusive, and including all numbers there between. In embodiments, an RdRp of this disclosure exhibits improved fidelity (i.e., a lower error rate as determined by incorporation of mis-paired nucleotides) relative to a control RT, such as an RT from a retrovirus. In embodiments, a method of the disclosure comprises cDNA generation using a one-step RT polymerase chain (PCR) reaction, or a two-step RT PCR reaction. In embodiments, a method of the disclosure comprises separating cDNA from a reaction in which the cDNA is produced. In embodiments, the sequence of the cDNA is determined. The cDNA sequence can be used for a wide variety of purposes, such as new virus discovery, or analysis of ssRNA or dsRNA viruses from any suitable sample. Thus, the disclosure provides compositions and methods that can be used for analysis of viral outbreaks, and for vaccine design, such as in the case of influenza viruses. Accordingly, in certain aspects, the RNA that is used as a template for producing cDNA may be present in a biological sample before the cDNA is generated. The biological sample can be any suitable sample, and can be used directly, or subjected to a processing step prior to cDNA generation.

The disclosure includes expression vectors encoding any RdRp or segment thereof described herein. Any suitable expression vector can be used, and many are commercially available and can be adapted by those skilled in the art to express an RdRp described herein, when given the benefit of this disclosure. Cells comprising the expression vectors are also included, as are methods of making any RdRp described herein by expressing the RdRp in a cell culture, and separating the RdRp from the cell culture.

In another approach the disclosure provides a method of testing test compounds to determine whether or not they are candidates for use as reverse transcriptase inhibitors. The method generally comprises: a) contacting a plurality of distinct test agents divided into separate reactions chambers with an isolated or recombinant RT, an RNA template, and a reverse transcriptase reaction buffer, b) allowing the test agents to be in contact with the RT, and subsequently, c) measuring of cDNA produced, wherein determining less cDNA relative to a control indicates the test agent is a candidate for use in inhibiting reverse transcriptase activity of the RT.

### BRIEF DESCRIPTION OF THE FIGURES

FIG. 1. Purification of Pepper cryptic virus 1 (PCV1) and demonstration of RT activity A. lane M, molecular weight marker, 1, purified PCV1 virions. B. M, molecular weight marker, 1, PCR product from commercially available MMuLV-generated cDNA; 2, water control; 3, PCR product from PCV1-generated cDNA.

FIG. 2. Aggregated PCV1 particles (A), likely due to the use of polyethylene glycol (PEG) in the preparation. Improved preparation with no aggregates (B).

FIG. 3. Image confirming recombinant RT protein expression. Expression was performed using a commercially available vector sold under the tradename pSUMO Vector. The sequence adds a 6× His Tag to the protein and a so-called small ubiquitin-like modifier which can be removed by protease digestion after purification by a 6× His tag, which is also added to the protein during recombinant synthesis.

FIG. 4. RT-PCR products using Zea mays chrysovirus 1 as a template, and primers for the RdRp gene. The following were used as reverse transcriptase: M, marker lane. 1, PCV1 virions; 2, Pseudogymnoascus desctructans partitivirus-pa (PdPV-pa) virions; 3, MMuLV RT (New England Biolabs). Expected size band is ˜500 nt (upper band in gel).

FIG. 5. RT-PCR product from in vitro translated PCV1 RdRp, using PCV1 dsRNA as template and primers for the RdRp gene. M, marker; 1, 1 μl of in vitro translation product; 2, 2 μl of in vitro translation product; 3, MMuLV RT (New England Biolabs).

### DESCRIPTION OF THE DISCLOSURE

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

The disclosure includes all polynucleotide sequences (RNA and DNA) encoding the RT of this disclosure. Cells comprising such polynucleotides are also included, as are all methods of making the polynucleotides and cells. Every possible DNA and RNA sequence encoding polypeptides disclosed herein are encompassed by this disclosure.

The present disclosure relates generally to a discovery made by our analysis of persistent viruses that are low titer viruses found in fungi and many plants, including crop plants. They are not transmitted horizontally, and remain in their hosts for thousands of years through nearly 100% vertical transmission. Through sequence analysis of a member of the Partitiviridae infecting fungi we discovered a domain in the RNA dependent RNA polymerase (RdRp) that had similarities to an RT conserved domain. A comparison of RT domains is provided in the Table below. Our analysis revealed that all of the RdRp genes from known partitivirus sequences we examined, including those infecting plants and fungi, have these conserved domains. However, and without intending to be constrained by any particular theory, it is believed that there has been no previous analysis of the potential RT activity of these enzymes because to date they are all found associated with viruses with dsRNA genomes. It seems likely, and again without intending to be limited to any particular interpretation, that any potential RT activity in these enzymes has remained undiscovered because these understudied viruses are not usually thought to be very important, and because there is no apparent reason why a dsRNA virus would ever need an RT, let alone preserve an RT domain over such a long evolutionary history. In addition partitivirus-like sequences are found integrated into the genomes of some plants and fungi, although no mechanism for how they were converted to DNA has been demonstrated. These observations, taken together with our sequence analysis, lead to the present demonstration that a partitivirus RdRp does indeed function as an RT.

In more detail, Pepper cryptic virus 1 (PCV1) is a plant persistent virus in the family Partitiviridae. PCV1 is found in all Jalapeño peppers. Since the pepper host of PCV1 is easy to grow, and PCV1 is generally a relatively high titer persistent virus, we selected this virus for further studies. We also compared PCV1 from different cultivars of hot pepper, including the presumed progenitor of all hot peppers, chiltepin. Chiltepin can be found as a wild plant in Mexico, but it is also consumed locally as a spice, and it is grown in tended areas or in gardens. Given the lack of horizontal transmission of persistent viruses, it is most likely that PCV1 has been consistently infecting chiltepin and domestic hot peppers for thousands of years, diverging with the divergence of their hosts. For most RNA viruses, thousands of years of divergence would lead to changes in the genome that might render them too different to be confident of their common origin, but in PCV1 the difference between the virus in Jalapeño peppers and in chiltepin is only 3%. Once again without intending to be restricted to any particular interpretation, these observations indicate that the RdRp of PCV1 has unusually high fidelity (i.e., a lower error rate) which supports an expectation that RT fidelity of the PCV1 RdRp, as well as other RT's described herein, will be unusually high as well.

Additional description of the isolation, demonstration of RT activity, and recombinant production of a PCV1 protein comprising an RT is presented in examples and figures of this disclosure. Additional demonstrations of embodiments of the disclosure are also provided using Pseudogymnoascus destructans partitivirus-pa (PdPV-pa) RdRp. Further, specific and non-limiting examples of a wide variety of RdRP proteins that comprise RT domains are provided, and include members of distinct virus genera, but all in the same family (Partitiviridae). In particular, representative RdRP sequences from Group I, II, III, IV and V (genera Betapartitivirus, Alphapartitivirus, Deltapartitivirus, Gammapartitivirus, and unclassified partitiviruses, respectively) of the Partitiviridae family are provided.

Based at least in part on the demonstrations of making and using RdRPs from two distinct dsRNA viruses, it is contemplated that any RdRp that is isolated from and/or produced recombinantly and/or/derived from any dsRNA virus that has an RdRp which comprises an RT domain will be suitable for use as an RT in embodiments of this disclosure. In certain examples the RdRp that comprises the RT domain is from a dsRNA virus that infects fungi or plants. In embodiments the RdRp that comprises the RT domain is from an encapsidated dsRNA virus. In embodiments the RdRp that contains the RT is a member of one of the Amalgaviridae, Birnaviridae, Chrysoviridae, Cystoviridae, Megabirnaviridae, Partitiviridae, Picobirnaviridae, Reoviridae, or Totiviridae families of plant or fungal viruses. In embodiments, the RdRp that comprises the RT domain is from a dsRNA virus that infects pepper plants. In an embodiment the RdRp that comprises the RT domain is from a dsRNA that is a PCV-1 or Pepper cryptic Virus 1.

In one non-limiting demonstration of an embodiment of the present disclosure, an in vitro translation of an RdRp and use of the translated RT to produce cDNA from a dsRNA template is demonstrated using a PCV1 RdRp that comprises SEQ ID NO:1, which is meant to be illustrative but not limiting. SEQ ID NO:1 is:

(SEQ ID NO: 1)
MVRGTLVGYDYTQFQGDLVKSTHRHPHVVHREIATTYVDQYAYEHIET
FSSLYPELILKGWSRSYYLPEKHLAAVLNYSMPNVPASQLSQSLYRQA
IESAKNGFISLPRVKAFDVLTEMDQVPFKSSSSAGYNYTGRKGLIGDE
NHSRAISIAKAVLWSAIKDDGEGIEHVIRTSVPDVGYTRTQLTDLLEK
TKVRQVWGRAFHYILLEGLVAYPFIQTVMSHKTFIHAGQDPLISVPRL
LSDVALNCKWIYSLDWSQFDATVSRFEIHAAFDIIKSYVDFPNYETEQ
AFEITRQLFIHKKVAVPDGYIYESHKGIPSGSYYTSLVGSIINYLRIN
YLWRLLTGHPPQQCHTLGDDSLVGDNSYVNPQAIEEAANKLGWHFNPD
KTQYSTVPEEITFLGRTYVGGLNKRDLTKCIRLLVYPEYPVESGRISA
YRAKSIAQDAGGLSEVLNRIADKLRRIYGTASEEEVPIYFKRYVFGV.

This sequence is available under GenBank accession no. AEJ07890.1. Conserved RT domains in SEQ ID NO:1 are shown in bold, with highly conserved amino acids shown in enlarged font.

Conserved RT domains were identified by comparison of the PCV1 sequence to other viral proteins, as shown in the following Table:

| | | | | | | | | | | | |
|---|---|---|---|---|---|---|---|---|---|---|---|
| Ty3 RT | 106 | .[ 94]. | XADTFRDLR | | FVNVYLDDILIFSES | | P | .[4]. | KHLDTVLERLKN | ENLIVKKKXCKFA | 253 |
| PCV1 RdRp | 185 | .[150]. | INYLWRLLT | .[ 5]. | QCHTLGDDSLVGDNS | | Y | | VNPQAIEEAANK | LGWHFNPDKTQYS | 389 |
| CAEV RT | 210 | .[100]. | MQEILEDWI | .[ 6]. | QFGIYMDDIYIGSDL | .[1]. | I | .[4]. | EIVKDLANYIAQ | YGFTLPEEKRQKG | 370 |
| BLV RT | 41 | .[101]. | LQEPLRQVS | .[ 6]. | LLVSYMDDILYVSPT | | E | .[4]. | QCYQTMAAHLRD | LGFQVASEKTRQT | 201 |
| Tf2-1 RT | 455 | .[ 94]. | INTILGEAK | .[ 2]. | HVVCYMDDILIHSKS | | E | .[4]. | KHVKDVLQKLKN | ANLIINQAKCEFH | 604 |
| RTBV RT | 1128 | .[ 98]. | MQESFGDLK | | FALLYIDDILIASNN | | E | .[4]. | EHLKIFFNRVKE | VGCVLSKKKSKMF | 1379 |
| GrpII RT | 115 | .[133]. | LDGLEALLA | .[17]. | NYVRYADDFIITGES | | K | .[5]. | QVLPVVRRFMAE | RGLMLSPEKTKIT | 319 |
| Ty3 RT | 254 | .[ 1]. | EETEFLG | .[ 3]. | 264 | | | | | | |
| PCV1 RdRp | 390 | .[ 3]. | EEITFLG | .[ 3]. | 402 | | | | | | |
| CAEV RT | 371 | .[ 1]. | YPAKWLG | .[ 3]. | 380 | | | | | | |
| BLV RT | 202 | .[ 1]. | SPVPFLG | .[ 3]. | 212 | | | | | | |
| Tf2-1 RT | 605 | .[ 1]. | SQVKFIG | .[ 3]. | 615 | | | | | | |
| RTBV RT | 1380 | .[ 1]. | KEVEYLG | .[ 3]. | 1390 | | | | | | |
| GrpII RT | 320 | .[ 3]. | EGFDFLG | .[ 3]. | 332 | | | | | | |

In the Table the following abbreviations are used: Ty, gypsy-like element from yeast; PCV1, Pepper cryptic virus; CAEV, Caprine arthritis encephalitis lentivirus; BLV, Bovine leukemia virus; Tf2-1, fungal retrotransposon; RTBV, Rice tungro bacciliform virus (pararetrovirus); GrpII, Group II intron from E. coli.

In the Table, sequences for the amino acid segments for each viral RT or RdRp sequence, from left to right including consecutively the top and bottom panels for each virus are as follows: for Ty3 RT, SEQ ID NOs:4-8; for PCV1 RdRp, SEQ ID NOs: 9-13; for CAEV RT, SEQ ID NO:s 14-18; For BLV RT, SEQ ID NO:19-23; for Tf2-1 RT, SEQ ID NO:s 24-28; for RTBV RT, SEQ ID NOs: 29-33; and for GrpII RT, SEQ ID NOs: 33-38.

In another demonstration of a non-limiting embodiment, isolated virions of Pseudogymnoascus destructans partitivirus-pa (PdPV-pa) (a Group IV partitivirus), comprising an RdRp with an RT domain to produce cDNA from a dsRNA template is demonstrated. The PdPV-pa RdRp comprises SEQ ID NO:2, which is:

(SEQ ID NO: 2)
MEVSPFDPTPLDNVIEGSPLVDDSLLVPSSRTRGSSYDVIPEHFNSPGLT
EIARYGGYPVYSGGSNTDAWVRTSLKEFDRTMYENIYGYTRKPEGPQGMY
KSLLKFSEDKSTFHSLNRVQRRAMIGAIKKARTAFKLPWKREPLDWHEVG
QFLRRDTAAGATFMGKKKGDVMEEIYHEARWLGHRMKQDGREKFNPKKMR
FPPCLAGQRGHMSERDTPKTRLVWVYPAEMLCVEGFYAPQMYRDFMNDRH
TPMLNGKSSQRLYTEWCVGLREGEKLYGLDFSSFDSKVPSWLIRVAFDIL
RQNIEWSTFRGEKVSKREAQKWRNVWDAMVYYFINTPMLMPDGRMFRKRR
GVPSGSWWTQMIDSVVNYILVDYLTQCQTCQIRGLRVLGDDSAFRSCHDF
SLDQASADAAAVLMILNPDKCEVTLDPTKFKLLGTTYEDGHPHRETIDWF
KFALYPESSVSSIDVSLTRLVGLWLGGGMWDLHFCKFMDYFQTCFPCPLE
GWFSKDQRRWLEVIFSGKAPRGWTTKKSLFWHSIFYTYC.

When given the benefit of the present disclosure, conserved RT domains in any of the RdRp amino acid sequences presented herein can be identified using for use in embodiments of the invention, using for example, a domain identification approach described in Marchler-Bauer A et al. (2017), “CDD/SPARCLE: functional classification of proteins via subfamily domain architectures.”Nucleic Acids Res.45(D)200-3, the description of which is incorporated herein by reference. In certain instances an RdRp that comprises an RT domain is referred to herein as an RT.

The disclosure includes amino acid sequences that are at least 60%, 65%, 70%, 75%, 80%, 85%, 90%, 95%, 96%, 97%, 98%, 99% similar, or identical to amino acid sequences presented herein, and/or to the RT domain of such sequences, such as across the entire length of the RdRp protein, or the RT domain. Additional representative RdRp proteins that comprise RT domains are described below and are encompassed within this disclosure. In embodiments a protein of the disclosure has any of said similarities over a contiguous segment of the sequence that contains the RT domains. In embodiments the protein comprises or consists of any amino acid sequence described herein, or comprises or consists of an RT domain present within any such amino acid sequence. In embodiments, an RdRp and/or RT domain of this disclosure comprises one or more of its amino acid residues substituted with conserved amino acid residues. In certain examples more than one amino acid change can be included. Such changes can comprise conservative or non-conservative amino acid substitutions, insertions, and/or deletions, provided the modified sequence retains or improves on the capability to catalyze the reverse transcription process. In embodiments amino acids substitutions may be substituted with conserved amino acids identified in the RT domain alignment shown in the Table. In embodiments, the disclosure provides recombinant or isolated or modified proteins that comprise RT activity and are at least 90, 91, 92, 93, 94, 95, 96, 97, 98, 99%, or 100% identical to the sequence:

(SEQ ID NO 3)

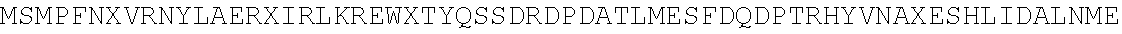

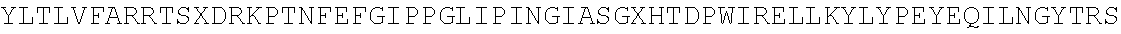

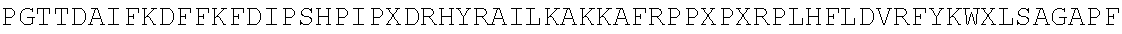

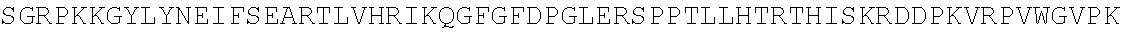

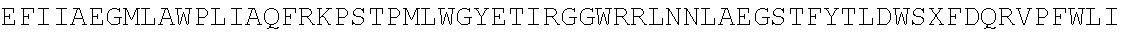

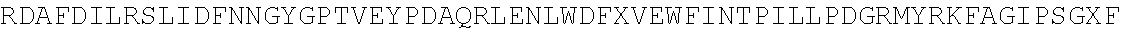

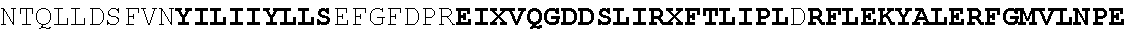

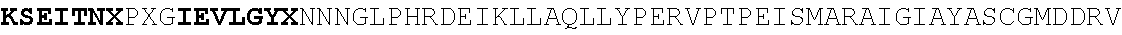

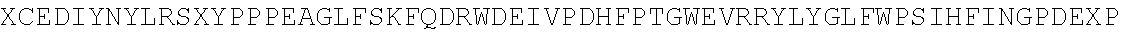

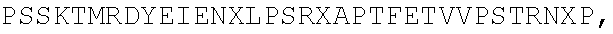

wherein X is any amino acid.

SEQ ID NO:3 is a consensus sequence of partitivirus RdRps. Residues in gray highlighting are conserved among all members of the family of RdRps that have RT-like domains. The residues in bold are domains conserved among a wide range of RTs. In embodiments, the disclosure includes an RdRp with RT activity that is from 415 to 756 amino acids in length, inclusive. In embodiments, a protein of this disclosure is from 600-650 amino acids in length, inclusive. In embodiments, a protein of this disclosure is from 550-610 amino acids in length. Proteins of such length can be at least 90% similar to any segment of any amino acid sequence presented herein. In one embodiment an RdRp with RT activity of this disclosure has at least 90% identity with SEQ ID NO:3 over its entire length, and in embodiments, includes any one or any combination of the amino acid sequences GDD, XRPL, SXFD, PSGX, wherein X is any amino acid, within the protein. In an embodiment, the RdRp with RT comprises the GDD sequence. In embodiments, an RT of this disclosure has at least 90, 91, 92, 93, 94, 95, 96, 97, 98, 99%, or 100% identity across the contiguous segment of SEQ ID NO:3 spanning amino acids 395-465, inclusive.

Any protein provided by this disclosure can be modified such as by being engineered to include a leader or secretory sequence or a sequence which can be used for purification e.g., a His-tag, and/or to include a proteolytic cleavage site. In a non-limiting embodiment the protein is engineered to comprise a ubiquitin segment and a protease cleavage site for removal of the ubiquitin segment. Modifications can be made at the N-terminus, C-terminus, or within the protein. Such modifications can be made using known reagents and techniques, given the benefit of the present disclosure.

In certain approaches the DNA or RNA sequence encoding a protein of this disclosure can be altered from a naturally occurring sequence, such as by optimizing codons for expression in any particular expression system. In certain approaches a polynucleotide sequence encoding a protein of this disclosure is modified by incorporation into any suitable expression vector, shuttle vector, plasmid, etc., as further described below and demonstrated in non-limiting examples. In certain approaches expression vectors, such as plasmids, are used. A variety of suitable expression vectors known in the art can be adapted to produce the proteins. In general, the expression vector comprises sequences that are operatively linked with the sequences encoding the protein, such as promoters, transcription initiation and termination signals, origins of replication, sequences encoding selectable markers, etc.

The disclosure includes methods of making the RT proteins. Such methods generally comprise either isolating virus that comprises the RdRp from the host where it is found normally and using isolated particles for performing reverse transcription, or by producing the protein recombinantly. Producing the protein recombinantly generally comprises initially introducing an expression vector encoding the RdRp into any suitable host cells by any method known in the art. Methods vary depending upon the type of cellular host, and include but are not limited to transfection employing cationic liposomes, electroporation, calcium chloride, rubidium chloride, calcium phosphate, DEAE-dextran, or other substances as will be apparent to the skilled artisan. In certain embodiments the cells used to produce the protein recombinantly are prokaryotes, including but not necessarily limited to E. coli, but eukaryotic cells may also be used, including but not necessarily limited to plant, fungal, insect and mammalian cells. The methods include allowing for a period of time during which the protein is expressed in the cells, and then separating the protein from the cells, after which the protein can be purified to any desired degree of purity. As discussed above, the proteins may be engineered to contain segments that improve protein expression, secretion, separation and/or purification.

In general, methods of this disclosure comprise combining incubating a fully or partially double-stranded RNA template, or a single-stranded RNA template with a purified or recombinant protein of this disclosure, such that a cDNA of at least one of the strands of the RNA template is produced. The method can optionally further comprise isolating the cDNA, and if desired determining or confirming the sequence of the cDNA. The RT and the RNA template can be combined in vitro to perform one or a plurality of cDNA assays. The RT could also be used to produce a cDNA copy of an RNA within a cell.

In certain embodiments, the disclosure includes generating a cDNA as described herein and determining the sequence of the cDNA. The sequence of the cDNA can be recorded in any tangible medium of expression. In embodiments, a plurality of cDNAs are determined and are compared to one another and/or to a database. In embodiments, the disclosure includes determining the sequence of a cDNA produced from dsRNA or ssRNA from, for example, a virus, and deducing an amino acid sequence encoded by the cDNA. In embodiments, cDNA sequences from cDNAs produced according to this disclosure are from, for example, retroviruses. In embodiments, cDNAs produced according to this disclosure are from samples comprising members of Reoviridae, such as rotaviruses, which comprise dsRNA genomes. In embodiments, cDNAs are produced from samples comprising influenza virus, including Influenza A, B or C viruses. In embodiments, cDNAs produced by amplification of all or a segment of an influenza virus single stranded RNA molecule are used to deduce the amino acid sequence of hemagglutinin (HA) and/or neuraminidase (NA) encoded by the viral genome. Thus, embodiments of this disclosure are suitable for generating genetic information that may be useful in predicting viral outbreaks, and/or for vaccine design. Furthermore, generating cDNA as described herein may be useful in diagnostic applications, such as to provide information about a viral infection in a human, or a non-human animal, or a fungus, or a plant, or a protist, or a bacteria or archaea. Further still, population studies (and other experimental analysis) of RNA viruses are limited by the error rate of the enzyme used for analysis, such as previously available RTs. These studies are critical for understanding virus evolution, including projected evolution of human pathogens such as influenza A virus, that is required for deciding on the specifics of future vaccines. Thus, the lower error rate that is expected to be produced by using an RdRp comprising an RT function as described herein provides advantages that are applicable to numerous applications.

Using an RT of this disclosure to produce a cDNA can be advantageous in a variety of techniques wherein determination of the presence, absence, amount, nucleotide sequence of a strand of an RNA, or combinations thereof would be useful. In this regard it is expected that any RNA can be used as a template for cDNA production using approaches of this disclosure, and there will be no particular upper limit to the bp length or other constraints on nucleotide composition, other than those imposed by the availability of reagents (i.e., dNTPs) to continue the reverse transcription process. In certain approaches, a fully or partially double-stranded RNA template is analyzed by cDNA production using an RT of this disclosure in a cell-free in vitro assay, or in an in vitro assay comprising cells. In the performance of such assays fully or partially double-stranded RNA templates will be in physical association with an RT of this disclosure, thus forming a complex comprising the RT and a dsRNA. The dsRNA may be in physical association with more than one RT of this disclosure at any particular time, and depending on the length of the dsRNA template distinct RTs may be concurrently generating cDNAs from the same dsRNA template in an anti-parallel direction. Those skilled in the art will recognize that a dsRNA template that is being reversed transcribed by an RT of this disclosure may include a segment that is in physical association with the RT but is not itself double-stranded due to the presence of a transcription bubble, wherein the strand being reverse-transcribed is transiently separated from its complementary strand in order to catalyze cDNA synthesis. Thus, the disclosure includes complexes comprising an RT as described herein, wherein the RT is in a physical association with a fully or partially double-stranded RNA template, and wherein the RT is also in physical association with a DNA polynucleotide (e.g., a cDNA) that is being synthesized during the process via RT activity.

In one embodiment the disclosure provides for determining the presence, absence and/or the sequence and/or the amount of a segment of RNA that is in a dsRNA configuration by exposing a sample comprising or suspected of comprising dsRNA with an RT of this disclosure, along with suitable reagents that are typically employed in a Reverse-Transcription PCR (RT-PCR) approach, and after a period of time determining the presence, absence, and/or sequence and/or amount of the cDNA. In an embodiment, by comparison to a suitable control, the absence of cDNA is a basis for inferring a lack of dsRNA in the sample, whereas the presence of cDNA indicates the presence of dsRNA in the sample. In an embodiment the method comprises comparison of ssDNA in the sample with a control reaction that is performed using a portion of the sample and a standard RT, many of which are commercially available, and include Moloney murine leukemia (M-MLV) and Avian Myeloblastosis Virus (AMV) RTs. Determining a difference in ssDNA relative to the control permits determining ssDNA production that it is attributable to the RT component of the RdRp acting on a dsRNA template, whereas the standard RT would not use the dsRNA as a template. Determining the sequence of a cDNA, if present, provides for identification of the sequence of at least one strand of dsRNA that was the template for cDNA synthesis, which also permits deduction of its complementary strand. Generating and determining the sequence of cDNAs is well known in the art, and the present disclosure includes performing this process, but with substitution of the RT of this disclosure for any of the well-known commercially available reverse transcriptase enzymes typically employed in RT-PCR reactions.

Generating cDNAs using an RT of this disclosure can be performed in a one-step RT-PCR, or a two-step RT-PCR. As will be recognized by those skilled in the art, a one-step RT-PCR reaction entails generating a cDNA using the RT, and PCR amplification of the cDNA in a single reaction container. Two-step RT-PCR entails the reverse-transcription reaction being performed in a single reaction chamber to obtain single-stranded cDNAs, which are then separated from the reverse-transcription assay and PCR-amplified in a separate reaction chamber. In certain approaches specific temperature parameters are included to avoid denaturing the RT. In certain approaches the methods of this disclosure include reverse-transcription cDNA generation at a temperature of less than 50° C. In embodiments, cDNA generation is performed at a temperature of less than 40° C. In embodiments, cDNA generation, i.e., producing cDNA by an RT of this disclosure, is performed at a temperature of from 10° C., to room temperature, wherein room temperature can range from 20-25° C. In embodiments, the only application of heat to generate a cDNA according to this disclosure is to temporarily denature dsRNA structures for the purpose of annealing primers. In this regard, those skilled in the art will recognize that at room temperature, currently commercially available and other previously characterized reverse transcriptase enzymes, would not be able to synthesize cDNA from a dsRNA template because the dsRNA renatures, which is not a suitable template for a non-RdRp reverse transcriptase. The kits of this disclosure described below can be adapted for either approach. Further, these approaches can be tailored for quantitative purposes.

In embodiments, an RT of this disclosure has a reduced error rate for nucleotide incorporation into the cDNA, compared to a control, such as a commercially available RT. In embodiments, the control comprises an error for reverse transcription by M-MLV virus reverse transcriptase. In embodiments, the control comprises an error rate for AMV reverse transcriptase. In embodiments, a control error rate ranges, from 1 error for every 17,000 bases to 1 error for every 30,000 bases. The error rate can be applied to a plurality of cDNAs. In embodiments, a cDNA of this disclosure can comprise from 500 nts to 12 kb in length. In embodiments, generation of a cDNA as described herein is performed without using any methyl mercury, such as methylmercury hydroxide. In embodiments, cDNA according to this method is performed without use of any reverse transcriptase obtained or derived from a bacteria, including but not necessarily limited to Thermus aquaticus. In embodiments, a cDNA generated according to this disclosure does not include an A-overhang. In embodiments, a cDNA according to this disclosure is generated without using any RNAase H.

It will be recognized from the foregoing that the present disclosure provides flexible approaches for detecting the presence, absence and/or amount of ssRNA or dsRNA by virtue of detecting the presence, absence and/or an amount of cDNA. Those skilled in the art will appreciate that as described above, this approach can be adapted to test any biological sample for dsRNA or single stranded RNA, which can be a critical step in detection and/or discovery of viruses, as well as other dsRNAs that may be present in a biological sample. Other dsRNAs from which cDNAs may be generated according to this disclosure can include double-stranded segments of RNA polynucleotides present in RNA secondary structures found in ssRNA virus genomes, mRNAs, tRNAs, snoRNAs, miRNAs, siRNAs, shRNAs, etc. Accordingly, in certain approaches an RT of this disclosure can be used for direct dsRNA sequencing and for instance, for RNA secondary structure mapping.

In certain approaches an RT of this disclosure can be used for screening a plurality of test agents to determine if they are candidates for use as an RT inhibitor, including but not necessarily limited to an RT that is a component of an RdRp. In general, the method comprises analyzing test agents using any system wherein the production of cDNA from dsRNA can be measured. In one embodiment, the method comprises screening a plurality of test agents to identify candidates for use in reducing RT activity by: a) contacting a plurality of distinct test agents (which may be divided into separate reactions chambers, such as in a high-throughput screen) with an isolated or recombinant RT of this disclosure in the presence of an RNA and RT-PCR reagents; b) allowing the test agent to be in contact with the RT for a period of time, and subsequently, c) measuring cDNA, wherein determining less cDNA relative to a control indicates the test agent is a candidate for use in inhibiting the RT. The presence and/or amount of the cDNA can be determined using standard approaches.

In an embodiment the disclosure provides a kit comprising an isolated or recombinant/modified RT as described herein. The RT can be provided as a single component or with other components, and can be included in or more sealed vials. The kit can include any reagents required for performing one-step or two-step RT-PCR, including but not limited to amplification buffers, one or more primers, nucleases, and dNTPs, including deoxyadenosine dATP, dCTP, dGTP, and dTTP. In an embodiment, an RdRp comprising RT activity is present in or provided with a buffer, such as a reverse transcriptase reaction buffer, which may comprise added dNTPs. The dNTPs need not include added nucleoside triphosphates that contain ribose as the sugar (NTPs). In embodiments, a buffer or other reaction component of the disclosure does not include uridine-5′-triphosphate (UTP) because the kit and other aspects of the disclosure are designed for DNA synthesis only. Thus, the kit and/or buffer includes dTTP instead of UTP. Accordingly, the disclosure includes buffers to which no UTP is added. In embodiments, the only nucleosides added to a buffer used in embodiments of this disclosure are dNTPs. In embodiments, the only nucleosides in a buffer or other reaction component of this disclosure are one or more dNTPs, and thus a buffer of this disclosure may include a nucleoside or nucleotide component which consists of, or consists essentially of, one or more dNTPs.

The dNTPs may be provided in any suitable molarity, such as from 1-20 μmol. In an embodiment, the dNTPs are provided in 8 μmol solutions. The kit may optionally include instructions for performing the RT that are either written on paper or in a computer-readable format.

In an embodiment, the RT is in a lyophilized form, and the kit further includes instructions for reconstituting the RT for use in cDNA production.

Additional representative RdRp amino acid sequences that comprise suitable RT domains are as follows:

The Following Representative Amino Acid Sequences are from RdRps from Group I Partitiviruses (genus Betapartitivirus)

Atkinsonella hypxylon virus
(SEQ ID NO: 39)
MSTLLIPQDTIAHTFDEAVASESNLRIDEVPENYLERFIHPSEPENFEFYSLRDSDIPSKRIPKNGIQVFENLKYHTNSKD
NLYKDQPSSGPSPMRGVANIIREYFPQYLDDLRTWCRPKSSDDSIFNDFNHEQRITQPFTEERERRLLPLIDHFLGIKPYD
IVHYCDTRFYPWKLSTRADYFHNHSRDRKAHAAKSHPDFATGPTKKSYFINSHLFFDRSTVHNIKEYGFPFRPTTDSARNE
TLLDLWFKKVPTELLVRSHISKRDNLKVRPVYNAPMIYIRIECMLFYPLLAQARKRDCCIMYGLETIRGGMNELERISNAF
NSFLLIDWSRFDHLAPFTISNFFFKKWLPTKILIDHGYAQISNYHDHVHSFSAQAQSHGIPMISKEYQTPPEATVFAKKVL
NLISFLERWYRDMVFVTPDGFAYRRTHAGVPSGILMTQFIDSFVNLTILLDGLIEFGFTDEEIKQLLVFIMGDDNVIFTPW
TLLKLIEFFDWFAKYTLDRFGMVINISKSAVTSIRRKIEVLGYTNNYGFPTRSISKLVGQLAYPERHVTDADMCMRAIGFA
YASCAQSETFHALCKKVFQYYFAKTSINERLILKGRKAELPGMFFAYPDVSEHIRLDHFPSLSEVRILLSKFQGYLKETPF
GTIPTFSTPQTLRDQTQ
Cannabis cryptic virus Fedora
(SEQ ID NO: 40)
MPYNAVRNYLAERMIRTKREWQLYQSSNRDPESTLEHYQDSDYLRYYQNARFNLNQEERFRALNKEYSTLVEAFRTDNLRK
HQPYELHQPIPPDAAPIPDKRQPAPGIKLVPLMYHYGHVVHDTPSTTDDHTSNDDSQSAPSRLLKTTEFGYSIDKQIYDLV
CRRYPTYLSVINLYCRPLGTVDATFSDFNKEQIPSNPIDPDRKEHVLKHIFKFLDATPYLPVHFVDTQFCKTPLVTGTGYH
NRYSFKQKAHAKYSHPEEYATYPSSKGYFYNATYENARTLVHYIKQNGLPFEFDFNLSEESLTDEQIDLFVNRSNAFFNDY
PTLLFTRNHISKRTGPLKVRPVYAVDDIFIIIELMLTFPLTGQARKPSCWIRMARNHSGSNRYIDRLGRSYSTFFFINWAS
YDQPMPRVITDIYYTDFLRSLIVINHGYQPTYEYSFYPDLDEHKLYDRKNNLLFFLHTWYNNMTFLLPDGFAYRRSYCGVP
SGLYNTQYLDSFGNLFLIIDAMIEFGFTDSEIDGFVLLILGDDNTGMTQMHIHRISQFINFLEKYALERYNMVLSATKSVL
TTLRSKIETLGYQCNYGSPKRDIDKLVAQLCFPENGLKPHTMSARAVGIAYAAAGQDYVFHSFCQDVYNMFRSYYKPDARA
NLFFQRQVLQNLEDGIPDLATPVVPPFPSLFEVREMYSEYKGPLSFEPKWNKAHFINDPNDIPPFSKTMRDYEKEHNLPVR
VSPTFETVVPSTKNLP
Cannabis cryptic virus Hemp
(SEQ ID NO: 41)
MPYNAVRNYLAERMIRTKREWQLYQSSNRDPESTLEHYQDSDYLRYYQNARFNLNQEERFRALNKEYSTLVEAFRTDNLRK
HQPYELHQPIPPDAAPIPDKRQPAPGIKLVPLMYHYGHVVHDTPSTTDDHTSNDDSQSAPSRLLKTTEFGYSIDKQIYDLV
CRRYPTYLSVINLYCRPLGTVDATFSDFNKEQIPSNPIDPDRKEHVLKHIFKFLDATPYLPVHFVDTQFCKTPLVTGTGYH
NRYSFKQKAHAKYSHPEEYATYPSSKGYFYNATYENARTLVHYIKQNGLPFEFDFNLSEESLTDEQIDLFVNRSNAFFNDY
PTLLFTRNHISKRTGPLKVRPVYAVDDIFIIIELMLTFPLTVQARKPSCCEVIYGFETIRGSNRYIDRLARSYSTFFSLDW
SSYDQRLPRVITDIYYTDFLRSLIVINHGYQPTYEYSSYPDLDEHKLYDRMNNLLFFLHTWYNNMTFLLPDGFAYRRSYCG
VPSGLYNTQYLDSFGNLFLIIDAMIEFGFTDSEIDGFVLLILGDDNTGMTQMHIHRISQFINFLEKYALERYNMVLSATKS
VLTTLRSKIETLGYQCNYGSPKRDIDKLVAQLCFPENGLKPHTMSARAVGIAYAAAGQDYVFHSFCQDVYNMFRSYYKPDA
RANLFFQRQVLQNLEDGIPDLATPVVPPFPSLFEVREMYSEYKGPLSFEPKWNKAHFINDPNDIPPFSKTMRDYEKEHNLP
VRVSPTFETVVPSTKNLP
Crimson clover cryptic virus 2
(SEQ ID NO: 42)
MPFNAVRNYLNERMTRLKQEWKTYQSTDHDPIKILDTIQDPDYRRYLDNARFNSDNEMKHLMLNKEYSTLVEAYRTDNAKK
HQVYELHQPIPNDAAPVIQSRLPAKGIKLVPLMYHYGHVTHDPVSLTDANRSDDFETQVVPDSDAPTRFGYPIDVRIYNLI
CYRYSKYLEVINAYCRPIGTVNATFEDFNKEQIPSAPIDPKRKENVLSHIHKFLDTKPYLPLHFVDTQFCKTPLVTGTGYH
NRYSFKQKAHAKYSHPAEYATKPISKGYFYNATYENARTIIHFIKEYGLPFNVIRADDKSELTDSDVQKYINEANSFFNDY
PTLLFTRNHISKRDGPLKVRPVYAVDDIFIIMELMLTFPLTIQARKPSCCEVIYGLETIRGSNRYIEQIARDYSTFFSLDW
SSYDQRLPRVITDIYYTDFLRSLLVINNGYQPTYEYPTYPDLDEHKLYHRMDNLLHFLHLWYNNMTFLLPDGYAYRRTSCG
VPSGLYNTQYLDSFGNLFLIIDAMLEFGFTDSEIQKFILLVLGDDNTGMTTIPICRMFNFITFLEKYALERYNMVLSTTKS
VLTTFRSKIESLGYQCNHGSPKRDIDKLVAQLCYPENGLKPHTMAARAIGIAYASAGQDDMFHSFCQDVYNIFRSDYRPDD
RMNLHFKRQIFHNLEDGMPDLAPPIVPPFPSLYEIREMYAYYKGPLDFAPKWNYAHFMSDPDTTPPYSKTMRDYEAENNIS
SRVAPTFETVVPSTINLP
Ceratocystis polonica partitivirus
(SEQ ID NO: 113)
MPSFSANPTYQTLIDDIDFDVPIAHPFSVINTDLQPVDDEETVHDLGSKDFEFYKVVSDNLPPTRAPSIGIESLPNIRYHN
HSNDHRYRDQPPTGPPPMRGVRKIINDSFPQYLPYLKEWCRPKTSSDAIFEDFNQPQIPSIPLSYNRKQRILNLVNHFMGV
KPYDIVHFCDTRFYPWDLSKKADYFHNHSNARKRHAQTSHASTATGPTKKSWFINAHLFHDRSTVHNIKLYGLPFKPHSYE
ARNKILLELWFKKIPTELLVRSHISNPKKLKVRPVNNAPMIFLRIECMLFYPLLAQLRKQQCSIMYGLETIRGGMMEIESL
ATRFSNFMMIDWSKFDQTVPFTLVDMFYQDWIPTLILVDSGYAKIHNYNDHVHSFAAQARKLGVHGDSNLNEAPPEAAVFA
NKVENLLKFINTWFKEMVYITPDGFAYSRTFAGVPSGILCTQLIDSFVNLVVLIDSLFEFGFHESDIKSALILLMGDDNVV
FAPDKLSQLHSFFKFLPDYAKKRWNMKVNVDKSIFTTLRRKIEILGYTNNYGMPVRSLSKLIGQLAYPERHVNDSDMCMRA
IGFAWCAAASDSTFHDFCRKVFIYYYARVNVPIKDLVQSNASALPGMFFAYRDVHQHIKLDHFPSIEEVRQVLSKHHGYLT
EEPLWKYDFFLHPKP
Ceratocystis resinifera partitivirus (SEQ ID NO: 43)
(SEQ ID NO: 43)
MPSFSANPTYQTLIDDIDFDVPIAHPFSIINTDLQPVDDEETVHDLGSKDFEFYKVVSNDLPLNRAPSVGIESLPNIRYHN
HSNDHRYRDQPPTGPPPMRGVHRIINDSFPQYLPYLKEWCRPKTSSDAIFEDFNQPQIPSIPLQHSRKQKILKLVNHFMGV
KPYDIVHFCDTRFYPWDLSKKADYFHNHSNARKRHAQTSHASTATGPTKKSWFINAHLFHDRSTVHNIKLYGLPFKPHSYA
ARNKILQELWFKKIPTELLVRSHISNPKKLKVRPVNNAPMIFLRIECMLFYPLLAQLRKQQCSIMYGLETIRGGMMEIESL
ATRFSNFMMIDWSKFDQTVPFTLVDMFYQDWIPTLILVDSGYAKIHNYNDHVHSFAAQARKLGVHGNSNLNEAPPETAIFA
NKVENLLKFINTWFKEMVYITPDGFAYRRTFAGVPSGILCTQLIDSFVNLVVLIDSLFEFGFHESDITSALILLMGDDNVV
FAPDKLSQLHSFFLFLPGYAKKRWNMTVNVDKSIFTTLRRKIEILGYTNNYGMPVRSLSKLIGQLAYPERHGFDSDMCMRA
IGFAWCAAACDSTFHDFCRKVFMYYYARVNVPIKDLVQANASALPGMFFAYRDVHQHIKLDHFPSIEEVRQVLSKHHGYLT
EEPLWKYDFFIHPKP
Fusarium poae virus 1
(SEQ ID NO: 44)
MLANIRDYFHEKLTRLLYDHKIFQSNSKDPDLTLEAHHSSDIERIYKSIHYDFNRSPAPIDYEAQYQSIKHILEDKQSQQG
FPHEYYRLHESPIPDDRIPPSGIKLLPFEYKSMNVVTATPEVPESGFKIHPRIERLLRSKYPQYLQYVRKYTRPLGTTNAT
VSDFFKPQTPSQPVEPTRINHVMSHVMKKMAITPYLPLHFVDTQYDKRPLANGTGYHNRRSHEMNIHALFSHPKEYESKRT
SKGYYVNAFLESARSLIHWIKLYGNPFRHCPSDLAQSLREFFLQRPTMLFTRNHISDRDGILKQRPVYAVDDLFLTIESML
TFPAHVIARKPECCIIVIYGLETIRGSNQILDKIASDYKSFFTIDWSGFDQRLPWVIVKLFFTEYIPRLLVVNHGYAPTYE
YPSYPDLTTNDMVSRLTNLITFLATWYFNMVFVTADGFSYVREHAGVPSGMLNTQFLDSFGNLFLLIDGLIEFGSTDAEID
DILLFIMGDDNSAFTTWSITHLEQFVSFFETYALSRYGMVLSKTKSITTTLRHKIETLSYQCNFGHPRRPIGKLVAQLCFP
ERGPRPKYMSARAVGMAWASCGQDKTFHDFCRDVYHEFNDDRADLDESAYLHIQSHLPGYLKIDESVRQIVDFQVFPSQQT
VYHTVSRWKGPLSYQPEMGSCSLCQPT
Heterobasidion partitivirus 8
(SEQ ID NO: 45)
MLSKVRDFFHEKLSRLLLQHSIFQSNDKDEEQTLLENQSSDVKRIYNSIHYSFQTDDTQAAYEDQYQHIKHVLEDKDRLSN
FPAEFYLPYDTTTTPDNRVPPSGIDQLPYVYKRTNVVTATDEVPETGYPIQNRLLRLIRSRYPQYLPHVRTFTRPLGTTDA
TVSDFFKPQHPSRLVDPSRISHVMKHVMNKMAITPYLPIHFVDTQYDKRPLSTGTGYYNRRSHEANIHALYSHPKEYENKR
TSKGYYINAFLESARSLVHWIKSYGNPFRSKPADPRESLKKFFLQRPTMLFTRNHISKILGALKQRPVYAVDDLFLTLESM
VTFPAHTIARKIECCIIVIYGYETIRGSNVQLDRLAQRYNSFFTIDWSGFDQRLPWVIVLLFFTEFLPRLLVINHGYAPTY
DFPSYPDLTTEMMYQRLSNILSFLATWYFNMVFITADGFAYVRRFAGVPSGLLNTQFLDSFGNLFLIIDALIEFGAQDEEI
DSILLLIMGDDNSGFTIWSIARLEQFITFLESYALTRYGMVMSKTKSLVTVLRHKIQTLSYTCNFGRPLRPIPELVAHLVF
PEREFKPQFMSARAVGMAWASCAQDKTFHDFCRDVFYEYLEESVPVDNTNIAWIQSHLPGYLRVDPEVTKMIDLNVFPSFL
HVSQKLSRWQGPLSYQPKWDLAHFINQPDVIPDDSITMFEYMQEHSLSLDIQFDLFSA
Hop trefoil virus 2
(SEQ ID NO: 46)
MPFNSVRNYLEERSVRVKKEMMTYQSSNRDPDAILEQSQDPDYRRYYDNARYNPSNDLKYRMLSKEYSTLVEAYRLRNDDK
HQPYELHQPVPQDAAPVPSYRAPAPGIKPVPLMYHYGHVIHDPVSLTESAIDDDSDTLPDSDEPTVTHFGYPINKRIYDLI
CNRYPEYLSVISAYCRPIGTVDATFKDFNKEQIPSGPIDPNRKEEVLTHIFRFLDAQPYLPLHFVDTQFCKTPLVTGTGYH
NRYSFKQKAHAKYSHPEEYAQMPTSKGYFYNATYENARTLVHYIKEYGLPFNIHYTPEDVDFTEEQIQAYIDSANNFFNDY
PTLLFTRNHISKRDGTLKVRPVYAVDDLFIIIELMLTFPLTVQARKQSCCIMYGLETIRGSNHYIERLARSYSTYFSLDWS
SYDQRLPRVITDIYYTDFLRRLIVINDGYQPTYEYPTYPDLDEHKMYTRMDNLLTFLHTWYNNMTFVLADGYAYRRTYCGV
PSGLYNTQYLDSFGNLFLIIDAMIEFGFRTPEIDDFILLVLGDDNTGMTVISIDRIYDFITFLEIYALTRYNMVLSTTKSV
LTTLRSKIETLGYQCNHGSPKRDISKLIAQLCYPENGLKPHTMASRAIGIAYASAGQDYMFHSFCQDVYNMFRLDYKPDAR
TTLNFQRQIYHNLDDGIPDLATPVVPPFPSIFEVREMYSRYQGPLTYAPKWNFAHFINSPDVTPAHYKTMRMYEIENNITI
RPAPTFETVVPTTRNFP
Lentunula edodes partitivirus 1
(SEQ ID NO: 47)
MVFQQIRDFLSERKLRIQEEWRRYQKAVSRDDTELRQSDASDIRRMYEATRDQLTEQEKAAVLDREFQGLIDSMLLKNENK
KENFEFSSTSAFDGFPPNRVPISGIAGIPRRFHTGQIVSESNEVPESGYPLDTLIDTLITNKYPEYRYYVDKYTRPLGTTD
ATFKDFNKEQVLIEPLDSARKERVMMHVHERLATTPYLPIHFVDTQFCKLPLHTGTGYFQRHSFWTQTHSKYSRPEEYHDR
PTSKGYVMNAFLILARTAVHKIKVSGLPFDFDFDDFEDDNAAFNELAKYLDKFLNDHATMLFTRNHISQRDGKLKQRPVYA
VDDLFILIEAMLTFPLLVLARDPACCIMYGLETIRGSMIYIDQISRMFNSFATIDWSEYDQHVPRPITDVYYYEFLPDLIV
INHGYQPTYEYPTYPDLDEHAMYKRIDNLLFFLHFWYNNMTFVTADGYGYRRKHCGVPSGLFNTQYLDSFGNTYLIIDGLC
EFGCSDEEIRLFLIFVMGDDNSLMSYWTLERLIAFIQFFEAYAKMRYNMTLSRTKSTLTAVRSKIEMLGYQCNFGRPSRPI
GKLVAQLCYPERGLFRKFMSYRAIGVAYASAGIDVKFYKFCKDIYFTYLPYAVAASEFNFLRAATHLPGYLKAFDDVSDYI
NFEKFPTIYEIREVYSYYHGPLSYEPKWNRAHFINRPNIVPPSAKTVGDYRRENNLQPRQVPILPTD
Primula malacoides virus
(SEQ ID NO: 48)
MPFNAVRNYLAERLVRVRTELKNFTSSNREPDATLELSQDPDLRRYYDNTRYNSSNDAKYRTLNKEYSTLVEAYRTDNQQK
HQPYELHQPIPADAAPIIDKRQPAPGLKLVPLMYHYGHVIHDPDPDQPDQTKVYPLDSRIYNLIMSTYPSYLSVLHDYCRP
IGTVEATFNDFNTEQIPSAPIDEDRKQQILKHLFKFLDVKPYLPIHFVDTQYCKTPLVTGTGYHNRYSFKQRAHAKYSHPE
EYALKSTSKGYFYNATYENARTLVHLIKTYGLPFNMQFACPKSDLTDEQINLYISKANQFFNDYPTLLFTRNHISKRSGTL
KVRPVYAVDDIFIIIELMLTFPLTIQARKPSCCIMYGLETIRGSNHYIDRLARPYSTFFSLDWSSYDQRLPRVITDIYYTD
FLRSLIVINHGYQPTYEYPTYPDLDEEIKLYSRMDNLLYFLHTWYNNMTFLLPDGYAYRRTHCGVPSGLYNTQYLDSFGNL
FLIIDAMIEFEFTDDEISKFQLLILGGDNTGMTNLAIDRIDKFITFLETYALARYNMVLPKTKCILTSLRSKIVTLGYECN
YGSPKRDIHKLVAQLCFPENGLKAHTMSARATGIAYASAGQDIMFHSFCQDVYNIFRSDYKPDVRANLYFQRQFLNDLEEG
VPDLATPTVPPFPSLYEIRKLYSKYQGPLSFTPKWNSAHFINEPDSVPISAKTMRQFEEEFSIPLMTAATFETVVP
Pleurotus ostreatus virus
(SEQ ID NO: 49)
MSFLRIRDYFTERLKHLSRDWKIFQQSDSDPESTLATHLDSDIARLEHGIKSSLSDDQRQQAYEREYNRIHSALHDKARQD
GFPDEFYRSRSIDDLPDNRIPPSGIIPLPYEYHRSQVVISTEEVPETGFQIDPRIVRILRNKYPQYLPHVTKYVRPLGTTD
ATVKDFFKPQIPSDPIPEARKQRILDLVISFLACTPFLPLHFIDSLWDKTPLHTGTGYFNRHSFAARIHAMFSAPRLYERR
TTSKGYFINYFLETARSTIHNIKLHGFPFDPSKVPDLGSALRSFILKRPTMLFTRNHISDRDGNLKQRPVYAMDDLFIRLE
SMITFPLHVMARKIECCIMYGYETIRGSNRQIDKIASSFRSFFTIDWSGFDQRLPRVITDIFWSDFLERMIVISHGYQPTY
DYPSYPDLTPDKMFQRMDNILFFLHTWYNNLVFVTADGFAYIRTCAGVPSGLLNTQYLDSFSNLFLIFDGLIEFGCSDAEI
YQIFLLVMGDDNSAFTLWSIAKLEEFLSFFESYALRRFGMVLSKTKSVITVIRGKIETLSYQCNYGAPKRPLAKLVAQLCY
PERGPRAKYTSARAIGMAYAACAMDRTFHDLCRDIYYEFLDDSASPDEPFFFEHVQAYLPGILRTDESLSTQISLSSFPSF
LTVQQHISRWQGPLSYYPKWDRAHFINDPDVIPPSAETMAEYRSRNSIPRRDIPSLWQ
Red clover cryptic virus 2
(SEQ ID NO: 50)
MPFNSARNYLAERMIRTKQELMTYQSEDHNPDAILEKSQDPDYRRYYDNTRFDPSNEVKHRILNKEYSTLVEAYRIKNDRK
HQPYELHQPIPEDAAPIPESRVPAPGLKLVPLMYHYGHIVHDPVASESDSDDDNTASERPSKTSIPHFGYPVNKRIYDVIV
NVYPEYLKVIGEYCRPIGTVEATFADFNKEQIPSAPINIERKEQVLTHIFKFLDAQPYLPLHFVDTQFCKTPLVTGTGYHN
RYSFKQKAHAKYSRPEEYAKLPTSKGYFYNATYENARTLVHFIKQFGLPFNLQYAPEDADPTDEQVQSYIDTANSFFNDYP
TLLFTRNHISKRDGTLKVRPVYAVDDLFIIIELMLTFPLTVQARKQSCCIMYGLETIRGSNHYIERLARSYSTYFSLDWSS
YDQRLPRVITDIYYTDFLRRLIVINHGYQPTYEYPTYPDLDEHKLYSRMNNLLYFLHTWYNNMTFVLSDGYAYRRTHCGVP
SGLYNTQYLDSFGNLFLIIDAMLEFGFSESEIDNFILLVLGDDNTGMTVISIDRIYDFINFLEKYALIRYNMVLSPTKSVL
TTLRSKIETLGYQCNHGSPKRDISKLVAQLCYPENGLKPHTMAARAIGIAYAAAGQDPMFHSFCHDVYNLFRLDYKPDART
NLNFQRQIYHNLEDGIPDLATPVVPPFPSLYEVRHMYSKYQGPLSYAPKWNYAHFINDPDVTPPSPKTMRDYEIENDLISR
TAPTFETVVPATRNFP
Rosellinia necatrix partitivirus 1-W8
(SEQ ID NO: 51)
MVLTIIRDYLHEAQLRLKKEWQTFQKSDQESGYSDKLPTDYDLRRYYDSARDYDAEKHKTEEYQHNFALTHERYTQMNADR
NEPFEFYRPLEDNELPDIRFPAPGITVLPFRYHTGQIVETTDELPDSGFSLHPLIDYLTKTKWLHYRPYIDKYCRPLGTTN
ATFSDFNREQIPSAPIDETRKNMVLPLVIYFLNALPFLPIHFVDTRFCGTPKHTATGYFQRFSTFFRTHAYYARNKLYALR
PTSKGYFFNTVYEFSRTWMHHIKEHGYPFVPSHDALDNARQYRIFMQKHVTMLFTRNHISDRDGFLKQRPVYAVDDFFILC
ELMISFPLHVMARYPINGIKSCIMYSFETIRGSNRYLDSIARDFISFFTIDWSSFDQRVPRVITDIFWTDFLRQLIVINHG
YQPTYEYPAYPDLSEHDLYKRMNNLLHFLHTWYNNMVFVTADGFAYLRSAAGVPSGLLNTQYLDSFCNLFLIIDGLFEFGF
TQAEILSIVFFIMGDDNSGFTMMDIERLTQFIEFFESYALKRYNMVLSKTKSVITTLRSRIETLSYQCNGGNPKRPLGKLI
AQLCYPEHGPKDKYMSARAIGIAYAAAAMDEEFHEFCRDIYHTFLPYAAPIDEHTLSMATKHLPGYFKMLDNIASEIKFDS
FPTLEMVQDKYSRWQGYLSHKPKWNDAHFKFLPETVPNNIKTMTDYQLEHKLDTPVPHSLF
Rosellinia necatrix partitivirus 6
(SEQ ID NO: 52)
MSSVFNNVRNYLQERLQHVKREWQIYQATGASSSNMTEERIQQLQDLDTRRMFHTARNAFDTTTNTPGPVSRITEEQRRLI
YDAESDKIMNALRQHDEMRSQPFELFMTRPDSDPIPPNRIAAPGIWQSPLQFHTGQIVHADPSTTSVLHPDEHEDYAESYL
PGDTDQGYEIDPTIYELLTRKYPEYLPYAQQYCRPKGTTDGTFRDFNKEQKPCPPLDSDRKEHVLKHVFKRLAIDPYLPLH
FVDTQYCKLPLVTGTGYHNRYSYRQKAHASFSHPLQYGSRPTSKGYFYNATYENARTIIHNIKESGVPFNIHFAPEDRDIT
DTEIDEYRRKCNNFFDQYPTLLFTRNHISNRDGTLKVRPVYAVDDIFIIIEAMLTFPALVQARKPDCCIMYGLETIRGANH
KLDSLAQSFSTYFTIDWSGYDQRLPRAITDLFYTDFLRRLIVISHGYQPTYEYPTYPDLNEDNMYDRMDKLLKWLHLWYNN
MTFVTADGYAYRRMYAGVPSGLFNTQFLDSFANLYILIDGMIEFGFTDEEIDSFLLFVLGDDNSGMTNLSLARLHEFIQFL
EAYALTRYHMVLSHTKSVITALRNKIESLGYQCNFGLPKRDIGKLVAQLIYPEEIKIKYHTMSARAIGLAYASCAYDKTFY
NFCKSIYNIFLDYYEYDEKTALNLSRFLTTGQDDLTTQFSWKILPPFPTREEIRKQISFYHGPLDYAPKWNFAHFINKPDV
IPPSSKTMYDYEIEHSLRPQPAPTFVAR
Sclerotinia sclerotiorum partitivirus 1
(SEQ ID NO: 53)
MDSIRNIFQELRQTNVLEWKLFQKFGQTTTNPTYSHPDADLRRIQETFRDPHLERSQTQEFKLHYTDTYSGFRESDLSKNE
DFEFYKPIDPSSIPASRLPAPGISVLPIRTHAEQVITATEQVPETGYQLHPLLRHLIVSKYPLYMQHATKLCRPLGTTDAT
FDDFNREQKQYPPIEPELALRIVRIIIHLLYALPFLPLHYIDTFFCKMPLHTGTSYFYRHSYELRTHVAFSAPSEYENKQT
SKGYFFNAFTSWARTVAHRIKEFGYPFDPTQLTPSEITDKLRSFFMEHATMLFTRNHISDRDGALKQRPVYAMDTLFLHLE
AMITFPLHVLARSSRSAIMYSMETIRGGCARMDALASSCQSYLCIDWSSFDQRMPWIIVDLFFTLFLPLLLIISHGYQPTA
EYSEYLGLTPDKMFSRLFNIISFLRLWYYNCVFVTADGYAYVRRFAGIASGMLNTQYLDSFCNLFLMIHALLHFGCTNEEI
LDFIYFVMGDDNVILTQWTIDRLFSFLTFFESHSLSRFGMVISTKKSVITSLRSRIEMLGYQCNCATPKRPISKLVAQLCY
PEHGPNDKYMSSRAVGMAWASAGYDAEFFAFCKDVHTLFTPFAAPPSEQTTQTILKHLPGLFKMLDDVTEFTNPQAFPDIM
TVRNRYATWQGELSPDKKWSRAHFLRRPDDTPLPFQTMFEYMSEHGITFPEPQQLF
White clover cryptic virus 2
(SEQ ID NO: 54)
MPHNSTRNYLAERMIRTKRELMTYQSKDRDPDAILEASQDQDYRRHYDNARYDPSNEVKYRILNKEYSTLVEAYRLRNDRK
HQPYELHQPIPEDAAPIPKSRVPAPGLKLVPLMYHYGHVVHEPAHSDESDSDDNSDAPTRPVKESVPHFGYPVSKKIFDLI
VRVYPEYIKVINTYCRPLGTVEATFADFNKEQIPSAPINSKRRETVLKHIFKFLDTQPYLPLHFVDTQYCKTPLVTGTGYH
NRYSFKQKAHAKYSHPEEYAKMHTSKGYFYNATYENARTLVHFIKQFGLPFNLQYAPEDADLTDDQVQSYIDAANNFFNDY
PTLLFTRNHISKRDGTLKVRPVYAVDDIFIIIELMLTFPLTVQARKQSCCIMYGLETIRGSNHYIERLARSYSTFFSLDWS
SYDQRLPRVITDIYYTDFLRSLIVINHGYQPTYEYPTYPDLDEHKMYSRIDNLLYFLHTWYNNMTFVLSDGYAYRRTHCGV
PSGLYNTQYLDSFGNLFLIIDAMLEFGFSDAEIDDFILLVLGDDNTGMTVIPIDRIYDFINFLEKYALVRYNMVLSPTKSV
LTTLRSKIETLGYECNHGSPKRDISKLIAQLCYPENGLKPHTMAARAIGIAYAAAGQDFMFHSFCHDVYNIYRLDYKPDAR
TNLNFQRQIYHNLEDGIPDLATSVVPPFPSLYEVRHMYSQYQGPLTYAPKWNYAHFINDPDDVPPNPKTMREYEIENDLIS
RTAPTFETVVPATRNFP

The Following Representative Amino Acid Sequences are from RdRps from Group II Partitiviruses (genus Alphapartitivirus)

Amasya cherry disease-associated mycovirus
(SEQ ID NO: 55)
MDHLTSLFELFAITPKTQNNLQFVGIYHRPPHSVRANLRNVEKHKITVAHAMHKYLYPHEIDFVINQMRRSDVTEDAILAD
FFDNNVEPLEPVLDEHFERGLSAMLDAFRPPQKCLPAHIYDVQHHYPYKWQVNAEAPFSTDSYFLANRPTFRAVFERLESL
YTHLATDWHRRYGNKTDNDDFMNDHVPAKFGPMKETVFSWTHRWHHVIKSNFTDTAGLSKDYYFKNRYIFPMLLHTKTAIV
KKDDPNKMRTIWGCSKPWIIADTMLWWEYVAYAKLQPGATPMLWSYETFTGGWLRLNHALFSSYIRHSYITLDWKRFDKKA
YFCIIDKIFDGVETFLDFDNGYLPTKDYPDTKSTWTQERSTRLKRLFDWTKENFYHAPIVLPNGHMYVRKFAGIPSGLFIT
QLIDSWYNYTMLATILSAMGFDPRSCIIKVQGDDSIIRLSALIPPDAHDSFLTKVQELADYYFQSVVSVNKSEVRNELNGC
EVLSYRHRHGLPYRDELAMLAQLYHTKARNPSPEITMAQSIGFAYASFGNHERVRLVLHDIHEYYKLQGYTPNRAGLSLVF
GNSPDLMIPHYTLDHFPSLREIKMFLTNAEYVNEETNSRTWPLNHFLHLPCHRT
Arabidopsis halleri partitivirus 1
(SEQ ID NO: 56)
MKNTVVLEPLPSLARPIYGDTDPGRNPAYQSTVDHALRRLLTAEEFNIVVNGYRRSPWNEDALTADIEKLNSDYHHVNKDE
HYYKAIEHTKKLFTPKEKLRPVHFNDLRHYPWQLSTSIGAPFATSEKWKDYINQKYDGKLKSRDFKDLFKETHGVSLEPYM
IDRRLSKRNFYNEMFYINRINIHHIKDGWTTNPAGHDLRYWHTAHARQHLVEAGDEDKVRLVFGAPSTLLMAELMFIWPIQ
TSLLARGSSSPMLWGYETTTGGWSRLYNWAYSALPRFGAVATLDWSRFDKDARHTVITDIHDLIMRPMFDFNSGYHPTIIN
PRSNPDPQRLENLWNWMKNAILTTPLLLPDGTRLQFQHSGIYSGYFQTQILDSMYNCVMIFTVLSRMGFDLNSVAIKVQGD
DSLILLSHSYTFLQHSFLTTFAHHAAVYFGSTLNVKKSELLPSLEDAEVLRYRNHGMMPYREELQLLAMLRHPERTASLSA
LMARSIGIAYANCGNYTRVHHICEDIHNYLKGIGVKPDAFGLPGGLRFRKNYLPSYEEIDISHFPTWLETVERLLDPSRPL
LTNKHWPTTHFFGIPGES
Beet cryptic virus 1
(SEQ ID NO: 57)
MDYLTSAFNRITHWFNVPSNLEYIGTFSLPPGILRVNEVAISNHKKTLEHSFNKYLYAHEIKLITQDYRRSDIDEESILAD
FFSGDVEKFEVPFDEHVETGLRCMADAFRPPRLCRPAHILDVKHGYPYKWNVNAEPPFSTDEYFLNQRKTFGEFIRMHEYE
HIDKADFFRRHPNTESHDLIRTIVPPKFGYLKSMIFSWTRRWHHIIKEGFTESTGLHTTGYFYNRFIFPMLLHTKTAIVKQ
DDPNKMRTIWGASKPWIIAETMLYWEYIAWIKLNPSVTPMLWGYETFTGGWFRLNRDLFCGFLQRSFLTLDWSRFDKRAYF
PLLRRILYTARTFLTFDEGYVPTHSAPTHPQWDHTKAIRLERLWLWTLENLFEAPIILPDGRMYRRHFAGIPSGLFITQLL
DSWYNYTMLATLLSALGFDPKHCIIKVQGDDSIIRLNVLVPQDQHQNLMDNLVQLAVNYFNAVVNVKKSEFGNSLNGREVL
SYRNHNGFPHRDEIMMLAQFYHTKARDPTPEITMAQAIGFAYASCANNKRVLWALKDVYDYYKDLGYTPNRAGLTLTFGDS
PDLFVPEISLEHFPTETEIRRYLTSTSYLNEAQNARTWPRTLFINAPAQ
Carrot cryptic virus
(SEQ ID NO: 58)
MDYLTTAFNRITHWFTTPTDFEYIGNFSLPPGLLRVNDTAISNHKKTLEHSFSKYLCADEIHLITKEYRRSDIDLDSILDD
FFSGDVEKFEIPFDEHVETGLRCMADAFRPPRLCRPAHILDVKHHYPYKWNVNAEPPFSTDEYFLTQRKTFGEFIRMHEYE
HIDKADFFRRHPNHESHDLLQTIVPPKFGYLKSTIFSWTRRWHHVIKDGFKDSSGLQTNGYLYNRFIFPMLLHTKTAIVKH
NDPNKMRTIWGASKPWIIAETMLYWEYIAWIKLNPGTTPMLWGYETFTGGWFRLNHQLYCGMIKHSFLTLDWSRFDKRAYF
PLLRRILYPVRSFLTFDEGYVPTHAAPYHPQWNHEKALRLERLWLWTLENLFEAPIILPDGRMYRRHFAGIPSGLFITQLL
DSWYNYTMLATILSALGFDPKQCIIKVQGDDSIIRLTVLIPQETHERFMAULTHLATYYFNAIVNVKKSEVANTLNGREVL
SYRNHNGFPHRDEINMLAQFYHTKARDTTPEITMAQAIGFAYASCGNHNRVLWVLNDIYNYYKDQGYSPNRAGLTLTFGDS
PDLFVPEIPLDHFPTKKEIRRYLTASSYINEAQNARTWPRDLFINRPAE
Dill cryptic virus 1
(SEQ ID NO: 59)
MDYLTTAFNRITNWFTKPANFEFVGLYSLPPGLLRVNDTAISNHKKTLEHSFSKYLYEDEIHLITKEFRRSDIDLDSILDD
FFSGDVEPFEIPFDEHVETGLRCMADAFRPPRLCRPVHILDVKHHYPYKWNVNAEPPFSTDEYFLSQRKTFGEFISMHEYE
HIDKADFFRRHPNRESHDLLKTTVPPKFGYLKSVIFSWTRRWHHVIKDGFKDDTGLKTTGYFYNRFIFPMLLHTKTAIVKH
NDPNKMRTIWGASKPWIIAETMLYWEYIAWIKLHPSVTPMLWGYETFTGGWFRLNNQLFCGMIKHSFLTLDWSRFDKRAYF
PLLRRILYQVRTFLTFDEGYVPTHAAPYHPQWDHEKALRLERLWLWTLENLFEAPIILPDGRMYRRHFAGIPSGLFITQLL
DSWYNYTMLATILSSLGFNPKQCIIKVQGDDSIIRLTVLIPHENHLKFMERLTELATYYFNAIVNVKKSEVANTLNGREVL
SYRNHNGFPHRDEINMLAQFYHTKARDPTPEITMAQAIGFAYASCGNHKRVLWVLNDIYNYYKDKGYTPNRAGLSLTFGDS
PDLFVPEIPLDHFPTTKEIRRYLTCSSYVNEAQNARTWPRTLFLKDPAE
Diuris pendunculata cryptic virus
(SEQ ID NO: 60)
MEYLIADFSRITHFFRDTTNLTYSGTYHFHPRHPEVNLNAYEAHQRVLRSLMDTHLFPHEIQLITDELRRSDMTIEAILAD
FFANDVEYHEIPFDSHIEYGIKCMLDAFRPPKRCRPVHLLDVQHHYPYKWQVNAEPPFSTDTYFLDNLPTYRDFWNERTSS
FDKYVDPEELNRRLRHRNIENLLDTKTPAKFGFLKNTVFSWTRRWHHIIKDGFTDTTGLTSDAYLRDRFIFPMLLHTKTAI
VKKLDPNKMRTIWGVSKPWIIAETMLYWEYIAYVKQNTGATPMLWGYETFTGGWLRLNAALYTSHVRFSFLTLDWKRFDKK
AYFPLIYKILLGVRDFLDFDNGYAPTVDYPDTKSTWTPHKSQRLQNLWLWTIENLFNAPIVLPDGRMYKRRFAGIPSGLFI
TQLLDSWYNYTMLASLLSALSMNPKSCIIKVQGDDSIIRLGTLIPPSQHEAFLLKLHALADFYFKASLSLDKSEVRNSLDG
CEILSYRHIRGIPYRDEITMLSQFYHTKARNPTPEIAMAQAAGFAYASCGHHRRVYNYLESVYNHYAVQGYTPNRAGLSLV
FGNSPDLILPHFELDHFPTISEIQHYYTSSVYRNESQMSKIWPLDHFLYPPAET
Heterobasidion partitivirus 7
(SEQ ID NO: 61)
MEYLSNLFSRVLKITKTTNFEFVGTYHNQPSIPQVNQIAIDNHQRTIRVAMERYLTSDEFSLITTGYKRTSLDPATITDDF
FSGDIEPHDEPQDLASQLAIEAGLNAMQRAFCPPNPARPVHLYDVEWHYPYKWQVNAEVPFSTETYFLKLRKKFSDFYDAA
SKTWTHYVNPLDALRRYGPEPPFDTLNQVTPPKFGFMKELIFSFVHSWLHVIKSRFHSNAGYTHSNFLRQRFLFPMQLHIK
TALVKADQPNKLRSIWGVSKLWIIAETMIYWELIAYMKLNRGSTPMLWGYETFTGGWFRLNAELQSSHLRQSITTIDWSRF
DKRAYFWLIRKILFRIRQHLDFNNGYVGTKDYPSSPTDPDKLQALWEWTLEALFDSPIILPDGKMYKRRFAGIPSGLYITQ
LLDSWYNYTMLAAILTYLGLDPERCIIKVQGDDSIIRLYVLIPPSEHDNFLLKMQEVATHLFASRISDQKSEVRDDLNGAE
VLSYRNNRGLPYRDEIQMVAQFYHTKAKDPTPEITMAQAIGFAYAACGNNYRIHSLLEEVYNYYHEQGFTPNPAGLSIVFG
DSPDRPDYPIELDHFPTQQETQRFLLSTDYRNAEQDARTWPLLHFLHAPCSRS
Heterobasidion RNA virus 1
(SEQ ID NO: 62)
MDYLTGLFSRVLHISRKVTNFEFAGTYHYQPSIPQVNEVATENHKRTLRHSFRTYLTSDEYDKIVNGYKRTNLDPSTITED
FFSGDIEDHPEPTDFKSQLSIEYGLQCMIDAFKPPAPARVCHLYDVQWHYPFKWQVNAEAPFSTEKYFLDLRKKFGDFFDP
VTKLWTKYVNPLDALRRYGHTPPADTLNQVTPPKFGFMKNLIFSFVHSWQHVIKSRFTSNAGITHSNFLRQRFLFPMLLHI
KTAIVSFDAPNKLRSIWGVSKLWIISEAMIYWEYIAWIKLNPGSTPMLWGYETFTGGWFRLWRDLHTPGEDVTYITIDWSR
FDKRAYFWLIRKIFIRTRCFLDFTNGYVSTKDYPTSPTDPDKLQALWEWTIEAFFDSPIVLPDGSMFKRLFAGIPSGLFIT
QLMDSWYNYTMLAAILHYMGYDPRRCIIKVQGDDSIIRLYIQIPLHEHDLFLLRMQEVSDHLFGAKISFEKSELRNSLIGS
EVLSYRNIQGLPYRDLIKMLAQFYHTKAKDPTPEITMAQAIGFAYAACGNDFRIHELLRSVYDYYKAQGFTPNPAGLTVVF
GDSPDRPDYPISLDEFPSQMDVQRFFLSTDYRNADQENRTWPSSHFLYAPCSRI
Red clover cryptic virus 1
(SEQ ID NO: 63)
MDYFISAFNRITHWFTTPTNFEYVGNYSLPPGLLRVNDVAIANHKKTLEHSFNKYLYSHEIKLITKEYRRSPIDEDSILED
FFSGDFEYFEIPFDYHVEYGIQCMADAFRPPRLCRPVHILDVKHGYPYKWNVNAEPPFSTDEYFLSQRKTFGEFIRMHEYE
HIDKDDFFRRHPNSESHDLLRTIVPPKFGYLKSMIFSWTRRWHHIIKSGFTESTGLETNGYFYNRFIFPMLLHTKTAIVKK
NDPNKMRTIWGASKPWIIAETMLYWEYIAWVKLNPGVTPMLWGYETFTGGWFRLNNELFCGLIKKSFLTLDWSRFDKRAYF
PLLRKILHTTRTFLTFDEGYVPTFAAPTHPQWNQEKAHRLDRLWIWTLENLFEAPIILPDGRMYKRHFAGIPSGLFITQLL
DSWYNYTMLATILSALHFDPKHCIIKVQGDDSILRLTTLIPVDQHANFMSHVVRLASHYFNSIVNVKKSEVANTLNGREVL
SYRNHNGLPHRDEITMLAQFYHTKARDPTPEITMAQAIGFAYASCANHNRVLWVLHDIYNYYHDLGYRPNRAGLTLTFGDS
PDLFVPEISLDHFPTKSEIRRYLTALHYQNEAQNARTWPRTLFINAPGE
Rhizoctonia fumigata partitivirus
(SEQ ID NO: 64)
MEYLVHAFKMFSLEPSVPQNLQMIGTYHYKPASPHVHTPHVEAHKKTVLKAMEKYLYPREINTIVNELRRSDVNLESILDD
FFDNNVPLHRIPFDEHVEYGLQCMADAFRPPRPARPCHLNDVEHHYPYKWQVNAEAPFATDKYFLDNRHKFRDFYDETTGT
WKHADPIDMERRYGNRLDTVLDQITPPKFGYMKNAIFAWTRRWHHIIKDGFTDLTGLVSSTYVRDRFIFPMLLHTKTAIVK
KDDPNKMRTIWGC SKPWIIAETMLYWEYIAWVKLNPGITPMLWGYETFTGGWLRLNAALFSSLMKNSFVTLDWKRFDKRA
YFELIYRIMLVARTFLDFENGYVPNVNYSATHTDWSHTKAQRIQRLWEWTLDNLFNAPIVLPNGDMYKRNWAGIPSGLFIT
QLLDSWYNYVMLATLLSALGHDPKACIIKVQGDDSVIRLAVLIPPQYHELFLTRLQDLADHYFGAVISIEKSEVRNELNGV
EVLSYKHIHGMPYRDEIAMMAQLYHTKARNPEPSITMAQAVGFAYASCGNHDRVYYALKDIYEYYASQGFAPNPAGLTLVF
GNSPDRFELDVPLDHFPTIHEIKKYLLNFEYRNEHQEHKTWPLDYFLNPPCSTL
Rosellinia necatrix partitivirus 2
(SEQ ID NO: 65)
MKNITDLPFPSKGVPQFRDALPIHGRDPEVPNPVTDASNRIIDFALRKHLTSDEFDQVVNGYRRSPWNEDALNKDIEKLDS
DEHTVIKDQHYENAIQHVQKLLTPEKPLQPVHFADLRRYKWRLSTNIGAPFASSKHWQDYVKAKFNHFRDGTPFENIAHRD
LFVEAHKDSQPLEITDARMTKHNLYTEAFYVSRETIHRIKDGETTDRYGNDARYWNTAFARQHLVKADEDDKVRLVFGAPF
SLLCAELMFIWPLFIFLLSLKGSTAFMLWTFETIIGGWYRLVNFFTTYALRHSTVVTVDWSGFDRYARHTVIKDIHHRIIR
PMFDFSHGYHPTRDYPDTSKTKDGQSNEWRITNLWNWMTDAILSTPLLLPNGRFIRFNHSGIYSGYFQTQILDSIYNLVMI
FTILSRMGFDLDKCVIKVQGDDSIFMLLCCFIMISTSFFTLFKYYAEYYFGAKLNEKKSEIRPSLQDAEVLKYRNRNGIPY
RDRISLLAQLRHPERRTDVDSVAARCIGIAYAACGQDATVYLICEDIYNYLVKKYDAHARQGELDFMFRHLELHDTVPSAA
TFPSWFDTMAHLTDGPRDPVPSHWPTDYFIGLPGRL
Rosellinia necatrix partitivirus 7
(SEQ ID NO: 66)
YNVQTAIDAVTEQFRPNRVLHPIQYPDLRYYPWTLNVSAEAPWTSYNFHFVPMDRSVDFESTQPKLIFDINQVNKLRKFSK
PTDVKTYLRWKQQVGLIENDHITFHNLYDEIFIYNRPLIHQIKEGEEPFWKDGQPVPYLWNTLHVRSHVVAHNEPDKLRGV
FGATKLVLQTEQPFIWPLQASYLNTDAGRLLWGREMSKGGWRKLFSEIYTFGPPSTVLSTDWSQFDKRLLHQLIRIVHRIW
RSYFDFTRYEPTNQYPNANPRDPKRLERLWDWMCNAITDTPILLPNGEIWRWQWNGFGSGYQQTQLMDTFANAIMIYTCLT
ALGVDVTNPKFWARFQGDDSLVAFFEQMFRIYGNDFLIMFSAVAEKYFNAKLNVKKSSILGQAHMATVLSYPNWHGTAFRT
DEDLLRHLMFPERPQDLGRLAASAIGLAQAALGCSERFHNLCEYIFTKLVKGKGVKVKWQALKWMVRAGQFETIEQLKRTE
FPTIEELLSQAQIPAIRTESERQRIWRTTPLSKDSFHFTHDI
Rose partitivirus PB
(SEQ ID NO: 67)
MRNTVVIGHRPVLAKPLFGNPDPGSNPAYGDTVDHALKRHLSPEEFDIVVHKYRRSPWNEDALKDDIAKLDSNEHPVLKDE
HYYRAIEHVKKLFTPEEKLRPVHFADLRYYPWQLSSNIGAPFATSKKWQEYVNDKFTAGQTAPQVRNLFQEAHGTPLEPEV
IDRRMTKRNLYNEMFLINRKNIHLIKDGRKTNDSGHDLRYWHTAFARQHLVEHDEPDKVRLVFGAPSTLLMAELMFIWPIQ
VSLLARGPDSPMLWGYETLTGGWSRLHTWATKAQPRLGSVLTLDWSRFDKDARHTIIKDIHSLIMRPMFTFDNGYHPTVYY
PETPETDPNRLENLWNWMTDSILTTPLILPDGKILQFMHSGIYSGYFQTQILDSMYNSVMLFTILSRMGFNIEKVAIKVQG
DDSIILMPYQYTVIKDTFLQFFSAYAQEYFGSTLNMKKSEILPSLERAEVLRYRNHGTMPERDELQLLAMLRHPERTSSLP
SLMARAIGVAYANCGNHTRVYQICEDIYKYLAKGGFKPDPFGLPGGLRYRQNYIPSYVQ1DISHFPTYFETVMHLQDPHRA
ILTNRHWPTDHFIGTPGKS
Rhizoctonia solani dsRNA virus 2
(SEQ ID NO: 68)
MNLYNRVSALFANWFSSPSNLEFVGSYHHQPGTVPPNPSTQEAHKRFLHNVFKQHLFTYELDYIENEHRRSEATPEAIEND
FFANDVEPHDIPFDVHVEIGLQCMTDAFRPPVPCLPAHLNDVEHHYPFKWQVNAEPPFSTDSYFLDNRKLFSDYYDTESQT
WRGYVDPFEANRRYQHTKDKEGFLNQTVPAKFGFMKDTIFSWTRRWHHIIKEGFQTATNLSSTAYLRDRFIFPMLLHTKTA
IVKKDDPDKMRTIWGCSKPWIIADTMFYWEYIAWIKKHPGVTPMLWGYETMTGGWMRLNSQLFSSYLKKSYVTLDWSRFDK
RAYFKLILAIMCRVRTFLDFDNGYLPNVNYPDTRTDWSPNKAQRLERLWLWTLECLIKSPIVLPDGRMYIRHYAGIPSGLF
ITQLLDSWYNYTMICTILSSIGLNPKHCIIKVQGDDSIVRLGVLIPPEAHEAFLLALQSKADFYFKATISVDKSELGNSLN
NREVLSYRNYNGLPRRDEIKMLAQFYHTKARNPTPEIAMAQAVGFAYASCGTHQRVLDALEHVYTDYKDAGYTPNRAGLSL
VFGNSPDIQLPHYDIDHFPSIEEIKRFLTCNSYDNSVQMAKSWPTSYFISEPCERM
Spinach cryptic virus 1
(SEQ ID NO: 69)
MDYLTSAFNRITHWFTKPTNLEYIGDYSTPPSIIRVNEVAIANHKKTLRYAFEQYLYDHEYRMIVDEYRRSDIDQESILAD
FFSGDIEPFDIPFDEHVEHGLRCMANAFRPPRPCRPAHILDVKHGYPYKWNVNAEPPFSTDPYFLSQRKTFGEFIQMHEYE
HIDKADFFRRHPNTESHDFLTTVVPPKFGYLKSMVFSWTRRWHHVIKTGFQDLTGLENSGYLYNRFIFPMLLHTKTAIVKK
NDPNKMRTIWGAPKPWIIAETMLYWEYIAWIKLNSGKTPMLWGFETFTGGWFRLNNILFCGLIKQSFLTLDWSRFDKRAYF
SLLRRILYTARTFLTFDEGYVPTHAAPTHPKWNHQKALNLERLWLWTLENLFEAPIILPDGRMYRRHFAGIPSGLFITQLL
DSWYNYTMLATLLSALGFDPKYCIIKVQGDDSIIKLTTLIPRDQHENFMVQLTALATTYFNATINVKKSEIRNTLNGCEVL
SYKNHNGFPHRDEITMLAQFYHTKARDPTPEITMAQAIGFAYASCGNNKRVLWILRDIYNFYKMRDVTPNRAGLTLTFGDS
PDVFMPEIPLDHFPTITEIRRYLTCSEYRNEAQDARTWPRSLFISGPAE
Sophora japonica powdery mildew-associated partitivirus
(SEQ ID NO: 70)
MASTNLLRLGKIPRSKKHNDPLSRSLRINRIRQGIIKRAIYKICPINLARQVIFGFKRSEGSDDVAETDFLRSDVPYFDMK
RDFHYLRALRVCERLFRPSRTLHPIAFPDLRFYPWSLSVSAEAPFSVEKKRSTLIRSRQSDGEDLDGRLTFHNLYNEIFEL
NRNLIHQIKEGDKSFWNKDGTPRPYWYNTLHTRPHLVKSSEPDKLRAVFGVPKLLLMAENMFIWSLQKEYLNQKIQSPMLW
GFETFKGGWLKIWNRMYSKKCSTFLSADWSGFDRFALFECVDDIHRMWRNWFDFSKYEPTIAESGPPGIQLSYPKSKTNPQ
KIERLWNWMCYCSKYTPIKGQSGQLYQWQYNGIASGYQQTQLIGSFVNSIYMLTCLSDLGINIESDNFQLFVQGDDSLTEF
SEIIQKDDLPKFLTNLAKVAKSRFNANLSVQKTTAGESLNDVEVLSYNNTYGIAFRDEAELLAHLLYPERFQTLEATVSCC
IGIAYASMGCSQYVDDTCLDAYNFLTTQFKVKPDSNFLQDFFRIRGSPLTDDFHNPRFPTKDECFNQNYEVRSRSDSEKQR
LWPSIPTGDYGFHFINE
Sclerotinia sclerotiorum partitivirus S
(SEQ ID NO: 71)
MPALNLRYLFSISRSEMKKIRSSHTKVSPREEATRFSILKHAILKHGSVGLLNQVLLGKRRSDASDERLIQDFHEFEQPVH
PVPRDKHYLRALRVTEKLMKPAKTLHPISFPDLRYYPWTKNVSAEAPYNFEKKYEELLRDKQRLGEIETATATFHNLEDEI
FEDNRYLIHKIKEGDSQFWDKDGKPRPYYHTTLHARAHVVGHEDADKIRAVFGVPKLLLMAENMFIWPLQAYYLNQDTTKH
HLLWGNEIMKGGWKKLWGQLQNGRISRTILSLDWSEFDKRALHEVIDDVHSMWKSWFDFTHYEPTIFYEKGEIPEPHRRIE
NLWIWMTDMVKHYPILQPDGKVYQWTRNGIASGFQQTQLLDSFVNMIMLLTVLSANGINIEHPDFWIKVQGDDSLISIVER
RFQMFGISYLDTLADLASYYFNAKLSVKKSFISDTPQGQYVLGYFNHYGIPYRLDDDLLSHLVFPERPQRLEETAASCVGI
AMASMGCSKVVYSICDDAYTFITKTLRRPAKAGSLFWLERAFGYEMPNIAKMPTFEECLYASYDIPVRTENMKQRLWPTNA
KAKNGFYFLRHLC
Vicia cryptic virus
(SEQ ID NO: 72)
MDYLISAFNRITHWFITPTNFEYIGYFSLPPGLLRVNDVAIANHKCTLERSFHTYLFDHEIKRIMIDHRRSDITEDSILED
FFAGDFPYFEVPFDEHVEYGLQCMADAFRPPRPCRPAHILDVKHGYPYKWNVNAEFPFSTDEYFLTQRKTFGEFIRMHEYE
HIDKDDFFRRHPNLESHDFLRTIVPPKFGYLKSTIFSWTRRWHHIIKLGFTDTTGLDNNGYLYNRFIFPMLLHTKTAIVKK
DDPNKMRTIWGASKPWIIADTMFYWEYQAWVKHNPGSTPMLWGFETFTGGWFRLNQLLFCGLIRRSFITLDWSRFDKRAYF
PLLRKIMYTVKSFLTFEEGYVPTHAAPNHPQWNQDKTDKLERLWLWTLENLFEAPIILPDGRMYRRHFAGIPSGLFITQLL
DSWYNYTMLATILHALGFNPSNCIIKVQGDDSIIRLNVLVPSERHDHLMSRIVELAEYYFNSIVNVKKSEIRNRLNGCEVL
SYRNHNGLPFRDEIAMLAQFYHTKARDPTPEITMAQAIGFAYASCATHTRVLWVLEDIYNYYRDQGYTPNRAGLTLTFGDS
PDLTMPEMPLDHFPTKSEIVRYLTCTNYRNEAQNARTWPRTLFINAPAE
White clover cryptic virus 1
(SEQ ID NO: 73)
MDYLITAFNRITHWFLTPTNLEYIGSYSLPPGLLRVNDVAVANHKATLDRSFDKYLYEHEINLITKEYRRSPIDEDSILED
FFSGDLPYFEIPFDEHVERGLECMAAAFRPPRPCRPAHILDVKHGYPYKWNVNAEPPFSTDEYFLSQRKTFGEFIRMHEYE
HIDKEDFFRRHPNIESHDFLRTVVPPKFGFLKSMIFSWTRRWHHIIKSGFQDSTDLEQTGYFFNRFIFPMLLHTKTAIVKK
NDPNKMRTIWGASKPWIIAETMFYWEYLAWIKHNPGATPMLWGYETFTGGWFRLNHELFCGLIQRSFLTLDWSRFDKRAYF
PLLRRILYTVKTFLTFEEGYVPTHAAPTHPQWSQENIDRLERLWLWTLENLFEAPIILPDGRMYRRHFAGIPSGLFITQLL
DSWYNYTMLATILSALHFDPLHCIIKVQGDDSILRLTTLIPVDQHTNFMDHIVRLADTYFNSIVNVKKSEVRNSLNGCEVL
SYRNHNGLPHRDEITMLAQFYHTKARDPTPEITMAQAIGFAYASCANHNRVLWVLEDVYNYYRDLGYRPNRAGLTLTFGDS
PDLTMPEMPLDHFPTKSEIRRYHTETHY
QNEAQNARTWPRTLFINAPGE

The Following Representative Amino Acid Sequences are from RdRps from Group III Partitiviruses (genus Deltapartitivirus)

Alternaria alternata partitivirus 1
(SEQ ID NO: 74)
MLLSEIPNVYQRALALERRLRLRELYPEGIIPYLARQEYAGSIAGPRGPSATVAPSVTTSF
IAKAPLSTYSKPEYTLECTETMQYLGKMDHYAFNNSVPKFDPWFRHVLKSKAPDVVLH
LEETYQRDPCTPERVMKFMKLFDRRWKRMPTGNVMKQAKEIVSEMFSKVGKVDPIDF
NYAGWHEILPHLDMSSSPGLPLRREYATQGECLGHIYDKTKRLNHFAKFLHPGAVRAPP
CMIGLRPGLIKKAEIDEKIKARGVWAYPAEVKVIEMRYCIPLMKRFSEMFGKTPYPVGR
NMTKALPFIIDHLLQDKKFGLVTDISKLDTSVGPDWIDWAFSQLKSFFDFGFTLSSERRD
SNVFDFLHFYFKRTPILLPSGQLVKKAGGVPSGSGFTQLVDTLVTTLATVYSRLRMGHT
KDQIFKMFVVGDDMATSVDHDFSIEEFSHIMGQLGFEINPSKVMFSNKGIELKFLGYSKR
GGGLYRPIEELLQTAFFPEKYVGNPNRSRQRILGQSIAAGLSNSFFDKCNYWMEELVTLS
STLDPDEVFIPQKRWMRNVLSIDEMPKSANVYDLFHLV
Beet cryptic virus 2
(SEQ ID NO: 75)
MRTINNYEYTSFTEDLEETDYTHPHVVRRDPEVTYEDTFAKKELLSRYPALYENLIRGW
SRSYYTGQEHLRAIMQYATPNTNFSECVQHAYTKAITKVTESLHSLPTVRAFNVLEELD
LIPYESSSSAGYTYRGVKGPQHGENHMQAIKTAKAVLWSVIKDDGEGIEHVIDTYVPDV
GYTRTQLTDLREKMKVRGVWGRAFHYILLEGTSAAPLLEAFANSNTFFHIGKDPTVSVP
YLISYTKGQAPWLTAIDWQAFDATVSRFEINAAFDIIKSKITFPNLETEQAFEISRQLFIHK
KLAAPNGKIYRIHKGIPSGSYFTSIIGSVVNRLRIEYLWNLKFNRGPKVCFTQGDDSLIGD
DELYSPMDMAAFVKPLNWFINTSKSMTSKVPEAITFLGRSSLGGLNQRDLKRCLRLLILP
EYPVTSGDISAFRANSIWRDSGSTSQILHEIANALRRKYGIAKEQDVPRYLRPWKA
Botryosphaeria dothidea partitivirus 1
(SEQ ID NO: 76)
MLLTEIPNVYQRALALERRIRVRELFPEGLVPYLKRQEHAGSIASPIGPSRSVAPKVTDKF
IAKAPLEDYPDKRYTLECTSTMQYIGQMDAYPFHSSIPEFDPWFRHVLKARAPDVSLHL
EQTYTRDPCTPDRVMSFLKLFDRTWKRKPTGRLMSQAESIVKKMFKCVGQVNPIDFNY
AGWHEILPHLDMSSSPGLPLRREYATQGECLGHIYDKSKRLNHFAKFLHPAAVRAPPC
MIGLRPGLLKKDELDEKIKARGVWAYPAEVKVIEMRYVIPLLERFKSQFGKIPYPVGVN
LTKALPFIIDHLLNDGKHGFVTDVSKLDTSVGPDWIDWAFSFLKDFYYMGMTESSETRN
SHVFDFLHYYFKRTPILFPSGQLVRKSGGVPSGSGFTQIVDTLCTLLMTTYSMLRMGYQ
EDDIIGKIFAVGDDMATSVPSSFDVEQFSFYVGQLGFEINVDKVMFSNRGIELKFLGYSK
YGGNIWRPIDELLQTAYFPEKYVGNPERSRQRILGQTLASGLTNGFLSKVNYWMEELCS
WHTELDTDEVYIPQKRWMRNVLGLDEIPRSALLFDIFPLC
Citrullus lanatus cryptic virus
(SEQ ID NO: 77)
MNISPLLYEQCLSGWSRSYYLHDKHMQAIIQYGYKDVPISSINDRLYKECVHEVQNRLS
SLPRVRALDVLSELDSVTFKSSSAAGYDYIGAKGPKGGENHTRAMSRAKAIMWSIAET
GETGMKHAIETAVPDVGYTRTQLADITEKTKVRHVWGRAFHYILLEGLTADPLIRAVQ
RADTFIHIGKDPTVSVPRLLSDTAEQCKWLYALDWKQFDATVSRFEIEAAFDIVLNLLDF
PNRETKLMFELSKQLFIHKKIAAPDGKIYWAHKGIPSGSYFTSIIGSIINRTEILMLWRTIT
GHGPIVCYTQGDDSLCGDNILIPPERFAMVANPIGWYFNQEKTEYSTIPELITFLGRSYAG
GLNKRDLKRCLRLLIFPEYPVESGRISAYRAQSISDDVGGLGDVLNKLADRLRRSIP
Diatom colony associated virus 14
(SEQ ID NO: 78)
MQEMTYSGVRYRTSNQGDYITKHPFGLSQIGSHKFTPVRRDPHTTIIDPFMMEAFQDYG
DRFNFAKLDGWSRSLYTREGHMDSIHRIQSHTRFHKPTDTSMTKTDDYCLQIFRTLGTV
RSLDYHTQLGQVPFEPNSAAGIGIPGKKGDSGNLALAINQAVATLQRSLRDGISSVIEDS
TPDMAYTRTQLTQLSAGIKVRNVFGQAFQYILLEGLSASPLMDHFVTNETFFFVGSDRPRI
SVPTLLEDFKKKGSLMMSIDWSAFDTSVENWEIVDAFNLLETILEFPNLETRAAFEFSRIL
FINRKIAAPDGNVYFKQKSVPSGSYYTMLIDSIINWRRILYLHHRATGFFPFDIRTQGDDS
LVATRDSVSPEALMLQIPRNSQWQLNPSKCPIGKSGSSVPFLQRTLKWGDQSRDLDRVE
RLAIYPEYEVESGDISAFRARALWEDCNYESVVLAHATSYLESKYGIPTTVPRRYTNIW
QTLFESKEREGLR
Fragaria chiloensis cryptic virus
(SEQ ID NO: 79)
MEHRFRGIPRGLIELEEIPTRRLREERVVHIDAWSSKAIDAIVPLSLRIELDGWARSYYTL
QAHIDSIMQYDRPKLPQPTNAAWNTTTQHVRTQFARMDKVQTLSYLQLDQVKWVRSS
AAGYGYVGRKSDNDNYFRARKTAFTIAEKLNHDRDYGPLALEDSTPDIAFTRTQLCQIK
VKRKIRNVWGEAFHYVLLEGLFADPIIQHFIRNKSFYFIGEDPLLAVPRLVEKILSEQDYV
YMFDWSGFDASVQEWEIRFAFSLLESILIFPSSVESYIWHFIIELFIYRKIAAPNGKVYLKT
LGIPSGSCFTNIIGSIVNYVRIQYLFFRLTNNFVTVFTHGDDSLVGVSTTQYVQMDNFEPI
CAEHNWTINIAKSAVSHEAGVSFLSRKVREHCHARDELLCLRMLKFPEYVVESGAMS
TLRAHSIHQDAGINSRYLYSIYKYLLHRYGKADSLPLNQQNWDPLEYENLRVSFATQNY
E
Fig cryptic virus
(SEQ ID NO: 80)
MEAGLIEIGNIPERHLRDEFIILVDQPAYDSVRRNAPQADMQEIDGWARSFYTVEGIMAS
IMQFSKPLIHEPTDPIWNDVKRETLMKIGSLFPQVQSLPFEGGFDHVPFESSSSAGYGYD
GKKGEGNNFHRAKSIANAAVRKFSEDIDNQGYDYAVSHLIQQGPDIAFTRTQLAKLPS
IKVRIVFGEAFHNILIEGLSAAPLLEAFKRMDTFYFTGKDPTIYVPRILHKMSTNEGWFIC
LDWKAFDASVQLWEIDHAFNCIQQLLAFPTELSRLAFLFTRESFKQRKLADPNGILWMR
KGGIPSGSYYTNIIGSVINYNRIEYVCKRLGLQKTSCYVQGDDSLIHITGDAKPDLTQLQ
MLGEQFGWTLNIPKCSLTQDSQLVTFLGRSQMHQLNIRERLKVLRLMCFPEYKVEDPKI
STTRVKAIARDAGWSDPVYNKIYLQLKRLYGEVERLPPHLATFVDRFDFQDVNM
Flammulina velutipes browning virus
(SEQ ID NO: 81)
MSDTLIDSFSRLTLSVKNFVFLGFTETQNYPQKSDSAILSHRKVVLNAFEKYLNPIEYNH
VANEYKRSETDLDSTKAAFFKGDIPDHEVPRDEHYNRAFSVIVSKFRPPEPIRPVHYADL
RLYPWPLKPSAEAPFSNDKSLLALLALRNRQGFLPNAKPNFHNLFNWVFGFNRQCVHLI
KKGKDNLGPNEYWPAHGFLYPINIHTKSAIIGIHDPNKVRTIFGVPKLTVMVEAMFFWP
LFRYYRFEQQSPLLWGYETMLGGWYKLNHELHLNPFYQGSILSLDWSFFDGRALFSVIN
DLYSDKGVKSYFEFNNGYIPTVDYPDSSTHPQKLHNLWDWMLTALKFAPCALADGTI
WQRTVRGIASGQFTTQFMDSIYNGLMILTILSRMGFVIDETLPIKLLGDDSVTRLAVSIPA
SMHESFLIEFQRLADYYFSHTINVKKSKISNTPHNVSVLSYANNNGLPVRSRTSLLCALL
YPKSRRPTWEHLKARAIGVYYASCGIDRTVRLICKDIFDYLDSQGIQASSAGLQDLFDPN
FKSGTIPLDVFPSVEQVTTNLRSFHHLDNSDKERYFPTSHFLDTK
Heterobasidion partitivirus 12
(SEQ ID NO: 82)
MQTLLSAVYSIRDAIFGKTLSEPHGLNVNFHFDGYVTDEIKTSIPPRVEMWYSDYQKFL
NPIIRANFTGAEADKIINGYHHPVATIPFMVENLKKGDLSDHPVPHDEHYLRARKMAAD
AFRPPRPIRPVHFADLRFYNWNWHPNVEEPYYSNKRAQEYVQAAHTLGLTPDARMSFG
NLRDYVFMDTRHYLHQIKRNEISNPKTLWPLMKIHVKPALTGTDEQKIRVIYGVSKRHV
LPQCMFFWPLFRHYIENDTDPLLWGFETILGGMMKLNSLMHRLYFQTFVTVDWSGFDL
RSLFSIQREIFDDWRTYFDFSNGYIPTVWYPNSTADPIQLERLWNWQRDACFNMPFVMP
DKSVYRRLFRAIPSGLFVTQFLDSHYNYIMLLTILSAMGFDITIERIRILVQGDDSLKNLIF
FIPANQHDNFKAEFQRLATYYFDHVARPEKTEIYNSPQGVTVLGYTNNNGFPTRDPIKL
MAQLYHPRQVGERWKSVLMAKCAGFAYASAYNYPKVTATLKTVYYKLAAKGFSPAA
LRTQRDIVLFGEAKFQVPTDHFPTAEEVQRHLRVPYKRTEEDSESYFPMKHFLDFA
Heterobasidion partitivirus 13
(SEQ ID NO: 83)
MLPIISQVTDYVYRKFIKPAPTQFKNNYLFQNWLSPLTTPHRDATKYAEYQAYLEKHIR
SNLLGSDAEYVIKRFHHPIATIDSVNETLQRGDLPDHPVPKDEHYYRALTETTKRFAPPQ
LIRPIHFADLRYYEWNWHPNVEEPYVSNSQLKTAVQDAYHAGLLEDGRMSFGNLKNH
VFMDVRHFLHRIKRGQISDPHTLWPLINMHWKPALTETDTTKIRLVFGVSKRHVLPRAM
FFWPLFRYYLDNRDKSPMLWGFETILGGMMLLNSEMLLSRLYYQTFVTVDWSGFDLRS
LFSIIREDIFPAWRTYFDFNNGYMPTKFYKSSTADPDQLERLWNWTNEAVFKMPFRTM
DGATFLRLFRGIPSGLFETQFLDSFYNMLMILTILDAMGFDISTIYIRVQGDDSLLLLTFFL
PADQHAEFKAQFEALAAYYFDHVARQDKTDISNTSQNVAVLGYSNDNGYPSRDWRKL
LAQLFHPRSQRPTLSLLKARCCGIQYASMYKYPQVTNVAKATFNQLDSEGVQPVKLAA
QRDVILHSHKDFYVPTDHFPTLNEVTRYLRIPYTRTEADSETYYPMSHFLSQF
Heterobasidion RNA virus 3
(SEQ ID NO: 84)
MKIFSTLYSSFASFAKWTGLTDPHGFEHNFYFTGYADKKIKVSINPRFQEVYDDYQSYV
GKFIDKHLPGHMAQKIKFGYHHPVASLPFMITNLKKGDLPDHPVPHDQHYTAARKAAA
DAFRPPRLVRPVHFADLRYYKWNWHPNVEEPYYSDPKLQQYVEHCYALGLIDDARLSF
GNLKDFVFMDTRHYLHLIKNGSITDNNQLWPIMKIHVKPALTEPTETKIRVIYGVSKRHI
LAQAMFFWPLFRYYIEEHTSPLLWGNETFTGGMLKIHNLISVPRLYSQTYLTVDWSGFD
LRSLFTIQREIFDDWRTYFDFTAYIPTRTYPDSKTDPIRMERLWNWQRDACFKMPFVLP
DRTTYARLFRSIPSGLFVTQFLDSHYNLIMIYTILSAMGFDITNLMILVQGDDSLIHLKFFL
PADQHDAFKAEFERLAKYYFDHIARPEKTHVTNSPNEVEVLGYTNNNGYPSRDMTKLV
AQLYHPRNVDKTSWKSLLMAKVCGFAYASCYQDSQVIDLLRSIYNNLASKGFKPKSGR
VMRDIILFGESEFEVPTDHFPTLNDVTKYFRRPYVRTQRDADSYFPSWHFNDVF
Pepper cryptic virus 1
(SEQ ID NO: 85)
MVRGTLVGYDYTQFQGDLVKSTHRHPHVVHREIATTYVDQYAYEHIETFSSLYPELILK
GWSRSYYLPEKHLAAVLNYSMPNVPASQLSQSLYRQAIESAKNGFISLPRVKAFDVLTE
MDQVPFKSSSSAGYNYTGRKGLIGDENHSRAISIAKAVLWSAIKDDGEGIEHVIRTSVPD
VGYTRTQLTDLLEKTKVRQVWGRAFHYILLEGLVAYPFIQTVMSHKTFIHAGQDPLISV
PRLLSDVALNCKWIYSLDWSQFDATVSRFEIHAAFDIIKSYVDFPNYETEQAFEITRQLFI
HKKVAVPDGYIYESHKGIPSGSYYTSLVGSIINYLRINYLWRLLTGHPPQQCHTLGDDSL
VGDNSYVNPQAIEEAANKLGWHFNPDKTQYSTVPEEITFLGRTYVGGLNKRDLTKCIRL
LVYPEYPVESGRISAYRAKSIAQDAGGLSEVLNRIADKLRRIYGTASEEEVPIYFKRYVF
GV
Pepper cryptic virus 2
(SEQ ID NO: 86)
MAIYRMLNGYVFTAFGNDLEKLDQRHIHHIRREEATTYRDEFALKELMDLSPILYEQFL
EGWSRSYYEGSKHLQAIIQYGIPDPDPGLIDNDIYNKAGYVVLESLGSLPRVRAFDVLTE
LDSVHYEQSSSAGYDYHGPKGPIQGENHIRAITRAKATLWSAIKDEGEGIEHVIRSAVPD
VGYTRTQLTDLYEKTKIRGVWGRAFHYILLEGTIANPLLDVFKRGGTFYHIGENPQYSV
PDILSQVSECCKYLVAIDWSNFDATVARFEINMAFDLIKTLIMFPNIETELCFEICRQLFIH
KKIAAPDGNIYWSRKGIPSGSYFTSIIGSIVNRLRVEYIFRKAYDVGPKMCYTQGDDSLIG
VDFRVDPDRLSEIAAPLNWKLNPAKTDVSLYPEHVTFLGRTMYGGINQRDLKRCLRLLI
FPEFPVPSGEISAYRAVSIAQDAGGTSEILNSIAKRLRRQYGVAEEHAVPKHFKLYIP
Persimmon cryptic virus
(SEQ ID NO: 87)
MALRSITGYEFHDFQSSLELLNQTHIHIVRRESGVTYHDEFALCELLVDNTRLYEQELEG
WSRSYYTGEQHMKAILQYSLPNTPIQDIDVGCYQQAMTNVQERLSSLPIVRAFDVLTQL
DQVSFESSSAAGYDYTGAKGPKNEGNHERAIRRAKAVLWSAIAQDGEGIEHVLRSSVP
DVGYTRTQLTDLSEKTKVRGVWGRAFHYILPEGTSADPLLQAFKEGGTFYHIGQDPTVS
VPYILSDTAGKCAWLYALDWSSFDATVSRFEIHAAFDLLKQRIEFPNFETEQCYEICRQL
FIHKKIAAPDGKVYWAHKGIPSGSYYTSIIGSIINRLRIEYIWIKLRGHGPTICYTQGDDSL
CGDDERIEPERIADIANPIGWLINPAKTATTRYPEYITFLGRTCYGGLNHRDLIRCLRLLIY
PEYPVPSGAISAYRANSIAEDCGGTSSILNDIARRLTRKYGRVSHEEVPKELRVYRH
Rose cryptic virus 1
(SEQ ID NO: 88)
MEHRFRGIPRGLIELEEIPTRRLREECVIHIDAWSSQAIDAIVPLSLRNELDGWARSYYTL
QAHVDSLMQYDRPKLQPPTNTAWNITTQYIRTEFARMKKVTALSYLQLDQVKWVRSS
AAGYGYTGRKSDGDNYIRARKTAFTLAEKLNHNRDYGPLALEDSTPDVAFTRTQLCQI
KVKRKIRNVWGEAFHYVLLEGLFADPLIQQFMRIKSFYFIGEDPLLAVPRLIEEILSEQDY
IYMFDWSGFDASVQEWELRFAFGLLESILIFPSSVEHQVWQFIIELFIYRKIAAPNGKIYL
KTLGIPSGSCFTNIIGSIVNYVRIQYMFFRLTREFVTAFTHGDDSLVGVPTTQYVQMENF
KPICDENLWTINIAKSAISREAEGVSFLSRKVREMCHARDELICLRMLKFPEYIVETGAM
STLRAFSIHKDAGIHSRYLYQIYKFLLHRYGKADSLPLNQQNWDPIEYENLRVSYATQN
YE
Rosa multiflora cryptic virus
(SEQ ID NO: 89)
MEHRFRGIPRGLIELEEIPTRRLREECVIHIDAWSSQAIDAIVPLSLRNELDGWARSYYTL
QAHVDSLMQYDRPKLQPPTNTAWNITTQYIRTEFARMKKVTALSYLQLDQVKWVRSS
AAGYGYTGRKSDGDNYIRARKTAFTLAEKLNHNRDYGPLALEDSTPDVAFTRTQLCQI
KVKRKIRNVWGEAFHYVLLEGLFADPLIQQFMRIKSFYFIGEDPLLAVPRLIEEILSEQDY
IYMFDWSGFDASVQEWELRFAFGLLESILIFPSSVEHQVWQFIIELFIYRKIAAPNGKIYL
KTLGIPSGSCFTNIIGSIVNYVRIQYMFFRLTREFVTAFTHGDDSLVGVPTTQYVQMENF
KPICDENLWTINIAKSAISREAEGVSFLSRKVREMCHARDELICLRMLKFPEYIVETGAM
STLRAFSIHKDAGINSRYLYQIYKFLLHRYGKADSLPLNQQNWDPIEYENLRVSYATQN
YE
Raphanus sativus cryptic virus 1
(SEQ ID NO: 90)
MSNLEYLGLDHHWPAIPRFAKSPNDWYFACQQLVRSSICLYSSLLGSNDTDTVLNGYY
RSHADEDTAEQFFMRYDVEPFDIIKDNYYSQAFDTVTEWFRPSAPIHPVHFTDVRWYP
WKISTSAERPFTHDPLLKKKVQLSKQLGLLDNARMSFHNCYNDIFTYCRHYTHEVKDA
RPVTLHHIDLHVKPALVRSGEPPKIRTVFGVPKSLIFAEAMFFWPLFSDYFTNSETPLLW
NYETLNGGWYRLNDEFYQQWQSFCTIFNLDWSEFDMRVYFSMLDDCRDAVKSYFCFC
GNYCPTRTYPTCRTNPQRLQNLWNWIGTAYKDTPCTTTTGKVYRRRFAGMPSGIFCTQ
FWDSFYNCIMVVTTLEALGFRITDRYFLKVLGDDVIFGILKHIPISKWADFLQDFSTEAR
RRFNSKLNSKKCGASSGIHGAQVLSYINWNGYPKRDSNQLLAQLLHPKSLRDTYPRLM
ARAIGIYYASCGDPKIRPICNHIYSELKYAGFTPSSTGLHGLFDPNASIGFIELDHFPSENE
VTCRLHRKSKRSAELQALYWPRDHFLEEAGSSRNCPLSFQVETI
Raphanus sativus cryptic virus 2
(SEQ ID NO: 91)
MDHEFRKIREGLIEIGTVSLRVQRDEFKVIIDEYAAEAVFKFVPSTMLSQLQGWARSVYS
LDQHVDAILAYRRQKLPEPTDDVWNQTKQHTLQLFRRFPKITPISYKSFDEVKWISSSSA
GYGYVGHKGDGDNYLKARRTAVTIAEKLDHDRNYAPEAINQSTPDVAFTRTQLSQVK
VKTKVRNVWGEEAFHYVLLEGLFADPLINFFSNEESFYFIGRNPLLSVPTLIEEIFKSKDYV
YAFDWSGFDASVQEWEIRFAFQCLESQLIFPSNVEAQIWRFIVELFIYRKIAAPNGTLFLK
TLGIPSGSCFTNMIGSVVNYVRIQYMFKKLTDDFVEAYTHGDDSLAAVSTAQYIPLEKF
GPICEPFMWSINTLKSEVSREGRLTTFLSRSIRDKQNYRDEFVCLRMLVYPEYEVEDGSIS
ALRAKSIYVDAGIHSQYLYHVFLYLKQKYGLANTLPHNLRTWDPTEHEALRASYSNIM
Rhizoctonia solani dsRNA virus 3
(SEQ ID NO: 92)
MVDALRKGDLPDHVIPKDEHYSKAFAQAAEMFRPPQLVRPVHFADLRMYKWNWHPN
VEEPFYSDADLIRAVSMAAEAGLLPDARMSFGNLRNVVFIKARLFLHQIKRKQITNPAT
LWPMMKIHVKPALTKVDETKVRIIYGVSKLHVMAQAMFLWPLFNYYINSDDDPLLWG
FETILGGMQKLHNIMSIPRLYFQTFVTVDWSGFDLRSVFSLQREVFDVWRTYFDFNNGY
IPTKFYRTSVADPDHLEALWEWQREACFKMPFVMPDRTMYNRLFRCIPSGLFSTQFLDS
HVNLVMILTILDAMHFDISKIKIYVQGDDSIVMLIFHIPADQHIKFKSDFEVLAKYYFDHV
ARPEKTDVYETPQGVEVLGYRNYNGYPERDWRKLLAQLLHPRGALSLETLAARCCGIA
YASMYRNPEVINVCKDIYNYLTTKRNVVPGELRAQRDIILFGEHEFSIPTDHFPERDEVT
RHLRIPYVRTDSDKNDYWPSGHFLSLY
Sinapis alba cryptic virus 1
(SEQ ID NO: 93)
MRPSITGYDYTNFTQDLLKSDRKHPHVVRRETATTYRDDFAFKEVISLDRLAYIQRLEG
WSRSYYLPEKHLEALLQYATPNVPCTALNLNVYRQAIQVVENGLRSLQPVRAFDVLTE
LNQISYKQSSAAGYDYIGAKGPIDGENHKRAISRAKAVLWSVVKEDGEGIDHAIETSVP
DVGYTRTQLADLTEKTKVRQVWGRAFHYILLEGLVAQPFIQSIMEGPSFIHTGRDPTLSV
PQSLAKVSSQCKYIYSLDWKSFDATVNRFEINTSFDIIKSKVIFPNYETEQAFEITRQLFLH
KKVAAPDGYIYEAHKGIPSGSYYTSMVGSIVNRLRIEYIWRIATGHGPIHCETLGDDSLC
GDDIFVPATQLADIANRIGWYFNADKTEYSTIPEGVTFLGRTSTGNLNSRDLTKCLRLLV
YPEYPVTSGRISAYRARSIADDSGGLSDLLNQVAIRLERSYGIASEEEIPAYFKRYVPFM

The Following Representative Amino Acid Sequences are of for RdRp's from Group IV Partitiviruses (genus Gammapartitivirus):

Aspergillus fumigatus partitivirus 1
(SEQ ID NO: 94)
MEDYTQDPTQHYVLAKGSHLIDALQLRPARSGKSSTTSYDVLPSNFESDTLREIARYGG
YSTYSSASNTDPWVRESLKIFDRDQYEAIRGFTRRPQGTPGMYESLKKFSTEERSTFWSL
SPQQRTSMRRAIGKAKRAFKLPYKREPLDWHEVGQFLRRDTSAGATFMGQKKGDVME
QIYHEARWLGHRMKQDGIGPFQPHKMRFPPCLAGQRGGMSERDDPKTRLVWIYPAEM
LVVEGFYAPLMYRDFMNDPNTPMLNGKSAQRLYTEWTCNLREGETLYGIDFSAFDAR
VPAWLIKAAFAILRQNVDFSTFRGKPVNKRDAQKWRNVWDAMVWYFINTPILMPDGR
MFRKFRGVPSGSWWTQMIYSVVNYIMIEYLADCQRVEIRNLRVLGDDSAFRSGDQFDL
DVAKGDAEPTQMLVNTDKSGKSKDPADFKLLGTTYRCGRVHRPTDEWFKLALYPESS
VFSLGLSFTRLIGLWLGGAMHDATFSRFIEFFQQCYPCPEEGWFSKDQRRWLEIVYSGK
APRGWTTKKSLFWRSIFYAYG
Aspergillus ochraceous virus FA0611
(SEQ ID NO: 95)
MDDSHLDPTQLENVIEESLLLDDSTLTPSAKVRGASYNVIPPQFSSPGLSEIARYGGYGT
YSGQSNTDPWVRVALKNFDRNVYDDVYGFTRKPEGTPGMYKSLFKFAEGRSDFRSLN
RAQRKAMQAAISKTKKRFKLPYKSDPLDWHAIGQFLRRDTAAGATFMGCKKGEVMED
IYHEARWLAHRMKQDGRQRFNPKQMRFPPCLAGQRGGMSEASDPKTRLVWIYPAEML
VIEGQYAPTMYHKFMADPHTPMLNGRSSTRLYTDWINDAKEGDKLYGLDFSSFDSKVP
SWLIRVAFNILRQNINFETWNGQPVSKRDRQKWRNVWDAMVYYFINTPILMPDGRMFR
KYRGVPSGSWWTQMVDSVVNDILVQYICLCQEIEPRDLRVLGDDSAFRSCAELVLVQA
ERDAKDVNMVLHPEKCDVKTDPTKMKLLGTTYRNGRAHRDTDEWFKLVLYPESSVRT
IEVSFSRLIGLWIGGAMFDSAFCRFMEYYQTCFQCPEDGWFSKEQRRWLEVVYGNRAP
RGWSAKKSLFWRSIFYAYA
Botryotinia fuckeliana partitivirus 1
(SEQ ID NO: 96)
MEEFTQEPTQHYVLAKGSHLIDALHLRPDTGKGSTTSEDVLSSDYRSPNLAEIARYGGY
STYSSNSNTDPYVRETLKLFSRDTYEDIRGFTRRPEGTPGMYKALEKFSGEKNSFNDLSA
TQKSSMRRAIGKAKKAFKLPYKREPLDWHEVGQFLRRDTSAGSTFMGQKKGDVMEEI
YHEARWLGHRMKQDGKGRFNPTKMRFPPCLAGQRGGMSERDDPKTRLVWIYPAEML
TVEGFYAPLMYRDFMNDPNSPMLNGKSAQRLYTEWCCKLREGETLYGIDFSSFDTKVP
AWLIRIAFDILRQNIEFSTFQGKPVSKKDAQKWRNVWDGMVWYFINTPILMPDGRMFR
KFRGVPSGSWWTQMIDSVVNHILIDYLADCQDVEIRNLKVLGDDSAFRSSDEFQLETAK
LDCKPTGMVIKPEKCEKTADPADFKLLGTKYRSGHVHRDTDEWFKLALYPESSVFTLD
VAFTRLIGLWLGGAMWDKRFCEYMDFFQSSYPCPEEGWFSKDQKRWLEVIYSGKAPR
GWTTKKSLFWRSIFYAYG
Colletotrichum truncatum partitivirus 1
(SEQ ID NO: 97)
MEDFTQDPTQHYVLAKGSHLIDALHLRPAKPGSTTSEDVLSSDFESPNLREIAKYGGYST
YSSNSNTDPWIRETLKIHDRETYEQIWGYTRRPQGTPGMYTALGKFAGEKNVFGDLSSS
QQSSMRRAIGKAKKAFKLPYKREPLDWHEVGQFLRRDTSAGSTFMGQKKGDVMEEIY
HEARWLGHRMKQDGKRSFDPTRMRFPPCLAGQRGGMSERDDPKTRLVWIYPAEMLV
VEGFYAPLMYRDFMNDRNSPMLNGKSAQRLYTEWCCNLREGETLYGIDFSAFDTKVP
AWLIRAAFSILRQNVNFETFQGKPVEKEEAQKWRNVWDAMVWYFINTPILMPDGRMF
RKFRGVPSGSWWTQMIDSVVNYILIEYLADCQKVEIRNLRVLGDDSAFRSGDQFSLESA
KIDCIPTGMIIKPEKCERTKDPSDFKLLGTKYHDLHPFRDTEEWFKLALYPESSVHTLDIS
FTRLIGLWLGGAMWDRKFCEFMDFFQTSYPCPEEGWFSKDQKRWLEVIYSGKAPRGW
TTKKSLFWRSIFYTYG
Discula destructiva virus 1
(SEQ ID NO: 98)
MEEFTQDPTLHNVQAEESHSIDTLHLRDAKRGSSTSEDVLHKGYADPCLREVAKYGGY
STYSSNSNTDPWIRETLKIHDRETYEDIWGKTRRPEGTPGMYKALGRFGGEKCDFDNLS
DPQKSSMRRAIAKAKKAFKLPYKREPLDWHEVGGFLRRDTSAGSTFMGTKKGDVMEEI
YHEARWLGHVMKQDGRKGFDPTKMRFPPCLAGQRGGMSDRTDPKTRLVWIYPAEML
VVEGFYAPLMYHDYMNDPKSPMLNGKSAQRLYTEWCCGLRDGETLYGIDFSAFDSKV
PAWLIRVAFDIVKQNINFETFEGKPVDKHDAQKWSNVWEAMVWYFINTPILMPDGRMF
RKYRGVPSGSWWTQIIDSVVNNILIDYLADCQQLEIRNLKVLGDDSAFRSTDQFDLEVA
KDDCVPTGMVIKPEKCERTEDPNDFKLLGTKYRDGRVYRSTDEWFTLALYPESSVLTL
DVSFTRLVGLWLGGAMWDKQFCAFMDYYQTSYPVPEEGWFSKDQKRWLEVVYSGK
APRGWTTKRSLFWRSIFYAFG
Discula destructiva virus 2
(SEQ ID NO: 99)
MEGFTQEPTNTTVLAEELHSVDTLHLRPGKTRSTTSEDVLPNNYEDPCLREIAKYGGYS
TYSSNSNTDPWIRETLKLHDRQIYEDIWGKTRRPEGTPGMYKALGRFGGERCGFDDLSS
QQKSSMRRAIAKAKKAFKLPYKREPLDWHEVGQFLRRDTSAGSTFMGSKKGDVMEEI
YSEARWLGHRMKQDGRSRFDPTKMRFPPCLAGQRGGMSDRDDPKTRLIWIYPAEMLC
VEGFYAPLMYRDYMSDPNSPMLNGKSSQRLYTEWCCNLREGETLYGIDFSAFDSKVPA
WLIRTAFDIVKQNINFETFEGKPVNKVDAQKWKNVWDAMVWYFINTPILMPDGRMFR
KYRGVPSGSWWTQIIDSVVNNILIDYLADCQSVKIPKPEVLGDDSAFRSNDQFDLEVAK
DDCVPTGMVIRPEKCEKTEDPAEFKLLGTKYRSGRVHRSTDEWFSLALYPESSVLSLDV
SFTRLVGLWLGGAMWDKQFCEFMDYYQTSYPVPEEGWFSKDQKRWLEIIYSGKAPRG
WTTKKSLFWRSIFYAYG
Gremmeniella abietina RNA virus MS1
(SEQ ID NO: 100)
MSEVDNTDPTLQDVAFVKSSGLDTTHLRSSEKTAGASYSVISSNFNSPGLTEIARYGGYS
VYSGNQNTDPWVRATLKNFSRETYEQIYGFTRQPEGVKGMYSSLLKFSDGKCKFDRLN
RVQRKAMIGAIAKAKKAFRLPYKSEPLDWHQVGAHFRRDTAAGVSFMGKKKGEVME
EIYHEARWLGHRLKQNGKARFDPRQMRFPPALAGQRGGMSKRDAPKTRLVWVYPAE
MLVVEGQYAPVMYRAFMDQPDTPMLTGASSQRLYTEWLVGRREGETLHGLDFSSFDT
KVPSWLIRVAFDILRQNIEWETWQGEKVSKRDRQKWRNVWDGMVWYFINTPILMPDG
RMFRKRRGVPSGSWWTQMVDSVVNYILVEYLTECQGVEARGLRVLGDDSAFRSPVEF
SLEQAQSDCEPTGMILKPEKCEKTEDPSDFKLLGTTYRGCHPHRDTNEWFKLALYPESR
VGNLEVSLSRLVGLWIGGAMWDKEFCSYMDYFQSSYPCPTEGWFSKDQRRWLSIVYG
GKAPRGWGDKKSLFWRSIFYTF
Magnaporthe oryzae partitivirus 1
(SEQ ID NO: 101)
MEKTSPDLPFTKDVLTSADWDAVHRSLSYGNPGLTKIPADKWCYKYNVEQTRMNTDP
FVRKAMKLWDETEYKQLYGYTKKASLEHGLNGLNKYGRPQRQKSHMPSEFKGSYHR
ALQEATRVFTPHEPLHRLSVPDVWDNMNLDSAAGFTFPGKKKSEVVEEAFDTASYMA
HFISAGKHIYVPPAKLALRGHLSELEEIKTRPVWVFPFEVTILEGKWAIPYYRFLEEEVPS
VHFGEGAMQRLAKILDSDIASHAEYAELTMDWSGFDTGVPNWMTDDALDILFGAFDE
TAVQHQDDLVVGGEYMAYKNEAVKDFLKTYFKKTKILLPDGSVYKKNHGIPSGSFFTQ
AIGSIINYIAVRTLDFYFGWNGRRFKVLGDDSSYLIPNGLGKVSVDAVSKAAWAAFGFT
LKREKLRIATKQHERKFLGYQVSAYRYERPSDDWLKMALYPERDCEFLEQSASRVFAF
YLLGGCNDATYCDFFHDYLNRYPVVYGSELFPLTKGLKRLFKFVLRLNVERLVFLDLPN
FDPLKGPFALSLGDKPFG
Mycovirus FusoV
(SEQ ID NO: 102)
MVELFVMVDPTTKRRRIQSTLGPFLSVPGLQEIARYGGYATYRAVRNTDPWIRQSLKLF
DPDLYGNIYGFTRRPAGPEGMYKSLMKFGESMPRFTDMSTVQRSAMKTAITAARKRFK
TPVKFEPLEWSEVGQHMRRDTSAGVSFPGKKKGDVMERIYAEGRWLGHRMKQGGKG
RFDPRKVRMPPCLATQRGHLSPRDDPKTRLAWIYPSEMLMVEGLYAPTMYKAFEAMP
DSPLLLGKGSHRLFSEWVSAATPGMRLYGLDFSSGDTKVPAWLIHTAFDILHDNIDWLH
WRGKPTTKRSRQKWKNVWDGMVYYFINTPILMPDGRMFRKRRGVPSGSWWTQLVDS
VVNWILVKYLSLCQGVNAKNLRVLGDDSAFMAAETMDLSVAAEDAAAVGMDLSDEK
SISVEDATELKLLGVRYRDGHAFRETEEWFKLALYPEGDVPDIATSLTRLVGLWIGGAM
WDTKFSRFMEYFQGCYPCPSEGWFSKDQRRWMEIVHGGRAPRGWTKNKNLFWRSIFY
TL
Penicillium aurantiogriseum partitivirus 1
(SEQ ID NO: 103)
MAFTQEPTQHYVLAKGSHLIDSLHLRPAKAGSATSEDVLPTGYNSPNLREIAKFGGYST
YSSASNTDPWVRETLKLFSRERYEEIYGFTRRPEGTPGMYKSLAKFAGEKSHFRDLTVS
QQKAMRRSIAKAKKAFKLPYKREPLDWHEVGQFLRDTSAGSTFMGQKKGDVMEDIY
HEARWLGHRMKQDGESSFNPTKMRFPPCLLAGQRGGMSERDDPKTRLVWIYPAEMLVI
EGFYAPLMYRDFMNDPNSPMLNGKSAPRLYAEWCCGLREGETLYGLDFSAFDTKVPT
WLIYTAFDILRQNIEWSTFQGKPVSKQDAQKWRNVWDGMVWYFVNTPILMPDGRMFR
KYRGVPSGSWWTQMIDSVVNYILIDYLAECQEVEIRNLRVLGDDSAFRSTDQFSLEQAK
VDCEPTHMLLKPEKCEKTKDPCEFKLLGYYTRDGRVHRPTEEWFKLVIYPESSVHTLDI
SFTRLIGLWLGGAMWDKEFCRYMDFFQSSYPCPEEGWFSKDQKRWLEVIYSGKAPRG
WTTKRSLFWRSIFYAYG
Pseudogymnoascus destructans partitivirus-pa
(SEQ ID NO: 2)
MEVSPFDPTPLDNVIEGSPLVDDSLLVPSSRTRGSSYDVIPEHFNSPGLTEIARYGGYPVY
SGGSNTDAWVRTSLKEFDRTMYENIYGYTRKPEGPQGMYKSLLKFSEDKSTFHSLNRV
QRRAMIGAIKKARTAFKLPWKREPLDWHEVGQFLRRDTAAGATFMGKKKGDVMEEIY
HEARWLGHRMKQDGREKFNPKKMRFPPCLAGQRGHMSERDTPKTRLVWVYPAEMLC
VEGFYAPQMYRDFMNDRHTPMLNGKSSQRLYTEWCVGLREGEKLYGLDFSSFDSKVP
SWLIRVAFDILRQNIEWSTFRGEKVSKREAQKWRNVWDAMVYYFINTPMLMPDGRMF
RKRRGVPSGSWWTQMIDSVVNYILVDYLTQCQTCQIRGLRVLGDDSAFRSCHDFSLDQ
ASADAAAVLMILNPDKCEVTLDPTKFKLLGTTYEDGHPHRETIDWFKFALYPESSVSSID
VSLTRLVGLWLGGGMWDLHFCKFMDYFQTCFPCPLEGWFSKDQRRWLEVIFSGKAPR
GWTTKKSLFWHSIFYTYC
Penicillium stoloniferum virus F
(SEQ ID NO: 105)
METTTPDLPFDLHTREAYDYATFHRTLLHKPGLSRIKEDRWVYKYNVEQTRMNTDPFV
RKSMKLWDEHAYHDMYGFTKKARLSNGLDAFQGFAKPQKQRSSMSPEMASCYEKAL
EEARHVFTPHERLTRLSVPNVCDSTNLDSAAGFSFPGKKKSEVVEEAFDVASYIAHFVA
SDRKVFIPPAKLALRGHLSEIDELKTRAVWVFPFEISILEGKWALPYYKFLEQNVPEVHF
GEGAMQRLAKTLMTDVASHSECTEVTLDWSGFDTSVSNWLIDDAFDIMFDSFDETQVE
HDGNFVLGGDHMAKKNEKVKKFLKTYFKKTKIMLPDGSLYKKFHGIPSGSFFTQIIGSI
VNYLAVKTLDNYFSWNARRFRVLGDDSSFLIPFGRSKVDGVEISEKAWETFGFTLKLKK
LRIANKQQDRKFLGYQCNAFRYERSTTEWLSMVLYPERDVEFLEQSASRVFAFYLLGG
CNDVTYCEFFHDYLGRYPYIYGKELPLTRGLKRLFKFVFRLTIDKLAFPDLSRFDPLKVP
FSLSLGDKPFW
Ustilaginoidea virens partitivirus
(SEQ ID NO: 106)
MEDFTQDPTHHYVLGKGSRLIDALHLRPAKEGQANSEEIVPSNFKSDTLREIAKYGGYS
TYSSNSNTDPHVREALKLFSRDIYEDIRGFTRRPQGTPGMYGALAKFSGERNAFSDLSAS
QQASMRRAFSKAKRAFKLPYKREPLDWHEVGQFLRRDTSAGVTFMGAKKGDVMEEIY
HEARWLGHRMKQDGRDSFDPTRIRFPPCLAGQRGGMSEIDDPKTRLVWIYPAEMLVVE
GFYAPLMYRDFMSDPNSPMLNGKSAQRLYTEWCCNLREGETLYGLDFSAFDTKVPAW
LIRVAFDILRQNIEFSTFQGKPVNKEDAQKWRNVWDAMVWYFINTPILMPDGRMFRKF
RGVPSGSWWIQMIDSVVNHILIDYLADCQDVEIRNLRVLGDDSAFRSSNQFDLEVAKQD
CVPTGMVIKPEKCERSEDPSDFKLLGTKYRGGHVFRPTEEWFKLVLYPESSVLSLDMSF
TRLIGLWIGGAMWDRKFCEFMDFYQSAYPVPEEGWFSKDQKRWLEVVFSGRAPRGWT
TKKSLFWRSIFYAFG
Verticillium albo-atrum partitivirus-1
(SEQ ID NO: 107)
MSDDIEIFDLLASPGEQPGSDETFASYGTPSSRTARRHVQAGEYFIGTGLEEIARYGGYSS
HRASGNTDPWVRETLKLYDPERYESIYGFTRTGEGLLGAYKSLFKFDGPVARGTRLSTR
QRSAMKKAIADARTAFKLPVKHEPLDWHEVGQFVNQSTSAGVSFPGKKKSEVMEEIYT
AARWLGHRMKEGGKESFNPTKVRFPPALAGTRGHMSPKDDPKTRLVWVYPAEMLVV
EGLWAPVMYRQYQSLSDGPLLLGKSAQRIYTEWCVNKKQGEVLHGLDFSGFDNGVPP
WIIHVAFDILHANVDWLNWRGKPTSKRSRQKWRNVWDGMKWYFINTPILMPDGRMF
RKHRGVPSGSWFTQLVDSVVNYILVKYAFNCQELKIHGLKVLGDDSAARSPLKLDLVQ
AAVDFRPVQMRLNLDKCEITEDATEFKLLGTRYQDGHSTRPDEDWFKMALYPENPPPD
IAVSMTRLVGLWLGGAMWSADFCKFFEYFQSSYPCPSEGHFSKDQRRWLEIVFGGSAP
RGWTYKESLFWRSIFYVF
Verticillium dahliae partitivirus 1
(SEQ ID NO: 108)
MEDFTQDPTTHNIVAEGSHLIDALHLRPPKLRSTTSEDVVPSKFRSPNPIDHDAMRGYST
YSSNSNTDPYVRETLKLFSRDTYEDIRGFTRRPQGTPGMYTALKKFSGERNTFGDLSPSQ
QSSMRRAIGKAKKAFKLPYKREPLDWHEVGQFLRRDTAAGATFMGQKKGDVMEEIYH
EARWLGHRMKQDGRAGFDPTQMRFPPCLAGQRGGMSEIDDPKTRLVWIYPAEMLVVE
GFYAPLMYRDFMSDPNSPMLNGKSAQRLYTEWCCKLRDGETLYGIDFSAFDTKVPAW
LIRVAFDILRQNVNFETFGGKPVEKRDAQKWRNVWDAMVWYFINTPILMPDGRMFRK
FRGVPSGSWWTQMIDSVVNHILIDYLADCQRVEIRNLRVLGDDSAFCSGGQFDLELAK
GDCENTGMVIKPEKCERTKDPGEFKLLGTTYRGGHVFRDTEEWFKLALYPESSVLTLDI
SFTRLIGLWLGGAMWDKKFCEYMDFFQSSYPCPEEGWFSKDQKRWLEIIYSGKAARG
WTSKKSLFWRSIFYAYG

The Following Representative Amino Acid Sequences are from RdRps from Group V Partitiviruses (Unclassified Members of the family Partitiviridae)

Fusarium poae partitivirus 2
(SEQ ID NO: 109)
MHSLFTIVNLLLLARFQLRRKRKETTKTLIYPIRNILFEWELRRSTPR
EPGIPLRYFDYSKSLLSYIDLQTCHKINIHLDDHTDALMERYASRDEP
FAVYQHISDEDLPPERTPAPGIRHAQCRYHEIPSGTLNLDEKQHVLTD
DPDFIETPEFRSGILYDETIDLSGSPPIPEIAQIIHDWFPHFEPFLAE
YCRPPSFGPQAFRDFNRPTPHPPPPPHERHEAIMDIVRAKFNLKPYRP
MHYVDALAAETPLNTSASYYSKFNPTSRVFARYSAPSRYKDKPTSKGY
NFNVVMNEFRTEYHHIKYDGVPFPADLHDPEANASILNTWFAKHPSQL
FIRTQISKRDPNDPKKIRPVYSVDDRFLHIEKTLVVPALAQLRNPQCC
VAHGLETFRGSMSLLDRTALVYTSYISLDWSQFDQRLPYYVIIAFFLD
FLPSLLIISHGYFPSRGYESTPQDIHAFASKIFNVVLFLTTWYLNMTF
VSFDGFAYIREHGGVPSGLLNTQFLDSFGNMYIIVDCLLEFGFTPAEC
LDMLYCVLGDDNLIFARQNFDRICDFMIFLTKYADTRHGMVVSILKSV
YTKLRSKISFLSYENTYGMPTRPIGLKVAQLSMPERPIPDNRKWIHAA
RALGLAYANCGQDAHFHLLCHMVYEKFRPDTPVPSLHIEKVFKKWKYQ
LPEFDIESVTYTFPDFPTLFSIRQLVSDYHGFFSETDKWNTDMFEAPP
SDNLHDYVTLKEYMNSNVHMSHTVNEFMHGKRSFL
Heterobasidion partitivirus 2
(SEQ ID NO: 110)
MSTNPPEVLLPLPEVDPNANNVRYAKLLDQYRANPSNSNQKLLLEYAE
QHGFNYFIPTDVPLPDDRKSAPGILSLDGFRFHTVAAFHRYQKMSRYG
YNALGFIFKLILTLFNPFLWILTDYCRPSGSADAVFENFNQEVSPVEH
VNPSRLAQIMPLIHHFFAIKPFCPIAFPDLRFYKWSLVTSADYHAHHS
KDQQDESAHYWKHLKDSDLLQDRFDYSDRPRSKGYFFNTVLLSTRTIV
HNIKYHCLPFQRHKRDTDSSVLQKLSFWFMKYPTVMYVRSQISKLSKL
KVRPVYNAPFLFILIEAMLTLALMAQCRLPDSCLMWGFETVRGGMQEL
NRISYNYDTFIMIDWSRYDQLLPFAIIYHFWCTFLPQLIRVDLGYMPT
EQYTATQHKHAFTEKHNDQKESNPEYATFASRLKTHAPHIVMFSFIIF
NLLAFIWLWYVKMVFVTPDGFGYVRLLAGVPSGIFMTQICDSFCNAFL
LIDAMLEFGFTPDDIKLIRMFIQGDDNVIFYLGDFTRIFAFYEWLPEY
CQQRWHMTISVDKSSITRLRNKIEVLGYTNLNGMPHRDCAKLIATLAY
PERYVQDKTKYIVFMSRAIGIAYANAGHDRQVHDLCQRAYLQARKDSG
LSYDELKNIKIEYQKLGFYEIFSVNIEELREHLIQDVSEFPNFHDIRD
NLRHWHGPHTVYPMWPRHFDDDLSSIKSPHSLTTLYDVMQSGGLTFDY
NF
Ustilaginoidea virens partitivirus 2
(SEQ ID NO: 111)
MNSFENFGSFKLSADELAATAVPPTPWNNVFRYITDAKRFPGYKRGIL
RQTQLYDPYVNAALKSFSPELHDSIKGYTRAPGDEWDVYERLTRYDKS
PLAPVDNPRFKACYDAALSDVMKEFKLRDPVVPHWILDVDLVKNTSSG
FPHFTRKGDILDQIRQEGRSHFHLLKRLPLWRVPLLPCTPATRGGLAD
ITEPKTRLVWMYPAAMTAVEAVFAQPLIDGLFSEKSEYLITGVDTKHR
IQRYLSLLSEDTGRLGVGLDFKSFDTLRCNWLIRDAFDVLKQNVYFSG
YYDDTNGLQTFGPGKTERLEHAWSNIVEYFIHTPILLPNGRCVNKHTG
VPSGSHFTNLIDSIICRILIKTFSLYCSIPISNLRTNGDDSAFHVYED
YASDIILRAAGFFKEFFGMTINTDKSCVAGSPSEMHVSGTRWTGLRPT
RSTQEWMMLAAYGETYSRIPFDSFQRLLGLGLSGGFGDSTFTRFFDYF
QTGYDCRHGPNLLNWKKLRFLQQIFSIEELPLVYKQGAKTTLRLRLLV
T

The following Examples are intended to illustrate but not limit embodiments of this disclosure.

### EXAMPLE 1

The RdRp of PCV1, and all known encapsidated dsRNA viruses, is found in the virus particle so the analysis described in these Examples began by evaluating enzymatic activity using purified virus. This Example provides a description of preparing isolated virus.

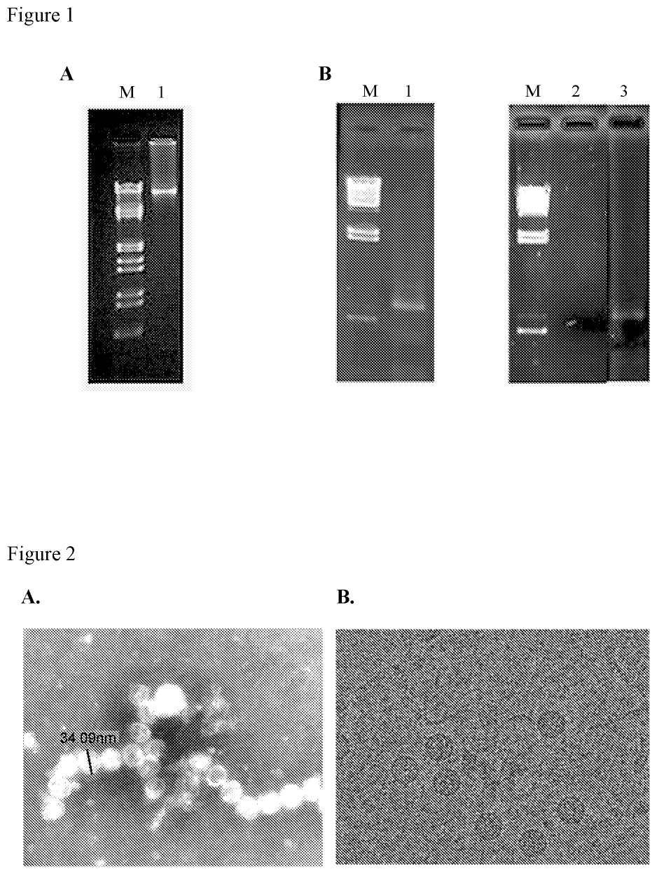

The procedure was standard for plant viruses. The virus was purified from 50 g of plant tissue by homogenization of plant leaves in a blender with 50 ml of 0.1 M sodium phosphate buffer pH 7.4 containing 0.2 M KCl and 0.5% 2-mercaptoethanol, and 50 ml of chloroform. The resulting slurry was clarified by low speed centrifugation for 15 min. The aqueous portion was filtered through miracloth, and subjected to ultracentrifugation through a 10% sucrose cushion. The pellets were resuspended in sodium phosphate buffer and stirred overnight. The solution was clarified by low speed centrifugation, followed by a second ultracentrifugation as above. The final pellets were resuspended in sodium phosphate buffer and allowed to incubate at 4° C. overnight. The purified viral preparation was analyzed by 1% agarose gel in Tris-Glycine buffer (FIG. 1A). Short term storage of the purified virus was at 4° C.; for long term storage sterile glycerol was added to a final concentration of 50%, and the preparation was stored at −80° C.

### EXAMPLE 2

This example provides a description of the RT activity of PCV1. Generating cDNA from dsRNA is not a straightforward reaction, because all known RT enzymes are only active on single-stranded RNA (ssRNA). Various strategies have been devised to use a dsRNA template, all of which require denaturing the dsRNA completely and adding chemicals, heat, and/or large amounts of oligonucleotides to maintain the template as ssRNA. Reactions are carried out at 42-55° C., temperatures that are well known in the art to be not optimal for RT enzymes. In particular, higher temperatures reduce the fidelity of the RT reaction (i.e. more errors are introduced in the cDNA sequence). In contrast, the present disclosure permits, as described above, cDNA generation at lower temperatures than have been previously possible, and without the use of denaturing chemicals.

To test the RT activity of PCV1, we replaced the MMuLV RT enzyme (commercially purchased from New England Biolabs) with the stored preparation of PCV1 in a cDNA reaction using the dsRNA of Zea mays chrysovirus 1 (ZMCV1) as a template. The reactions were adapted from standard protocols: dsRNA was mixed with a specific primer and boiled for 2 minutes, followed by rapid cooling on ice, addition of the enzyme, buffer, and dNTPs, and incubation on ice for 15 minutes. The MMuLV reaction was transferred to 42° C. for two hours, while the PCV1 reaction was held at room temperature for the same amount of time. At this temperature a “normal” RT enzyme would be unable to use dsRNA as a template because the RNA would have renatured. In a negative control, added water was used as the template along with PCV1 as RT enzyme.

Following the cDNA reaction, the samples were treated with 1 μl (10 mg/ml) of boiled ribonuclease A (Sigma, USA), and incubated at room temperature for 15 min, to destroy remaining RNA. The samples were heated to 85° C. for 2 min, and the primers removed using a cycle pure kit (Omega, Bio-Tek, USA) according to the manufacturer's instructions. The samples were eluted in 30 μl water. A 1.5 μl aliquot of the cDNA was amplified by PCR in a standard reaction with the same primer used in the cDNA reaction, and forward primer specific for another region of the cDNA. The amplified cDNAs were separated on a 1.2% agarose gel, stained and visualized (FIG. 1B).

A second reaction was carried with another dsRNA virus, Curvularia protuberata thermal tolerance virus. In this reaction we added an additional sample, where the starting dsRNA and primer were not boiled. We obtained the same product with and without boiling the dsRNA, further substantiating that the PCV1 enzyme is indeed able to use dsRNA as a template.

Following our initial success we prepared a much larger virus isolation, using about 1 kg of plant tissue. This is not a rapid process, because it takes some time to grow the plants, and this needs to be performed in a growth chamber to prevent the risk of exposure to environmental pathogens that would complicate the results. We also performed a virus purification procedure on an isogenic line of Jalapeno that is virus free to provide a control to ensure that the activity was completely attributable to the virus. Because of the large volume we concentrated the plant extract using polyethylene glycol, a standard procedure in plant virus purification. However, this prep had no RT activity, and an EM showed particles that were highly aggregated (FIG. 2A). A third prep with another kg of plant tissue was accomplished, and in this case we restored activity and the virus particles look normal by EM, as shown in FIG. 2B.

### EXAMPLE 3

This Example demonstrates recombinant production of an enzyme derived from PCV1 RdRp that exhibits RT activity. In particular, in order to avoid having to prepare virus from plants, the DNA sequence encoding the PCV1 RdRp was optimized for expression in E. coli and was cloned into a commercially available vector sold under the tradename pSUMO Vector. The sequence adds a 6× His Tag to the protein to facilitate purification, and small ubiquitin-like modifier (SUMO) that can be removed by protease digestion of the recombinant protein. The E. coli expression optimized DNA sequence is:

(SEQ ID NO: 112)
GGTCTCAAGGTATGGTTCGTGGCACCCTGGTTGGCTATGATTACACCC
AATTTCAAGGCGATCTGGTTAAAAGCACCCACCGTCACCCGCATGTTG
TTCACCGTGAGATCGCGACCACCTACGTGGACCAGTACGCGTATGAGC
ACATCGAAACCTTCAGCAGCCTGTATCCGGAGCTGATTCTGAAGGGTT
GGAGCCGTAGCTACTATCTGCCGGAAAAACACCTGGCGGCGGTGCTGA
ACTACAGCATGCCGAACGTTCCGGCGAGCCAGCTGAGCCAAAGCCTGT
ATCGTCAGGCGATCGAGAGCGCGAAGAACGGCTTCATTAGCCTGCCGC
GTGTGAAAGCGTTTGACGTTCTGACCGAAATGGATCAAGTTCCGTTTA
AGAGCAGCAGCAGCGCGGGTTACAACTATACCGGTCGTAAAGGCCTGA
TCGGTGATGAAAACCAACAGCCGTGCGATCAGCATTGCGAAGGCGGTG
CTGTGGAGCGCGATTAAAGACGATGGCGAGGGCATCGAACACGTTATT
CGTACCAGCGTGCCGGACGTTGGCTACACCCGTACCCAGCTGACCGAT
CTGCTGGAGAAGACCAAAGTGCGTCAAGTTTGGGGCCGTGCGTTCCAC
TACATCCTGCTGGAAGGTCTGGTGGCGTATCCGTTCATTCAGACCGTT
ATGAGCCACAAGACCTTTATCCACGCGGGTCAAGACCCGCTGATTAGC
GTGCCGCGTCTGCTGAGCGATGTTGCGCTGAACTGCAAATGGATCTAC
AGCCTGGACTGGAGCCAGTTCGATGCGACCGTGAGCCGTTTCGAGATT
CACGCGGCGTTTGACATCATTAAGAGCTACGTTGATTTTCCGAACTAC
GAAACCGAACAGGCGTTCGAGATCACCCGTCAACTGTTTATTCACAAG
AAAGTGGCGGTTCCGGACGGCTACATCTATGAAAGCCACAAAGGCATT
CCGAGCGGTAGCTACTATACCAGCCTGGTGGGCAGCATCATTAACTAC
CTGCGTATCAACTATCTGTGGCGTCTGCTGACCGGTCACCCGCCGCAG
CAATGCCACACCCTGGGTGACGATAGCCTGGTGGGTGACAACAGCTAC
GTTAACCCGCAGGCGATCGAGGAAGCGGCGAACAAGCTGGGCTGGCAC
TTCAACCCGGATAAAACCCAATACAGCACCGTGCCGGAGGAAATCACC
TTTCTGGGTCGTACCTATGTTGGTGGCCTGAACAAGCGTGATCTGACC
AAATGCATTCGTCTGCTGGTGTACCCGGAGTATCCGGTTGAAAGCGGT
CGTATCAGCGCGTACCGTGCGAAGAGCATTGCGCAAGACGCGGGTGGC
CTGAGCGAAGTTCTGAACCGTATCGCGGATAAACTGCGTCGTATTTAT
GGCACCGCGAGCGAAGAAGAAGTTCCGATTTATTTCAAGCGTTATGTT
TTTGGCGTGTAA

The optimized sequence comprises modifications to remove tandem rare codons that can reduce the efficiency of translation or disengage ribosomes from the RNA, by changing

GC content to prolong mRNA half-life, to disrupt some predicted stem-loop structures, and to remove negative cis-acting sites.

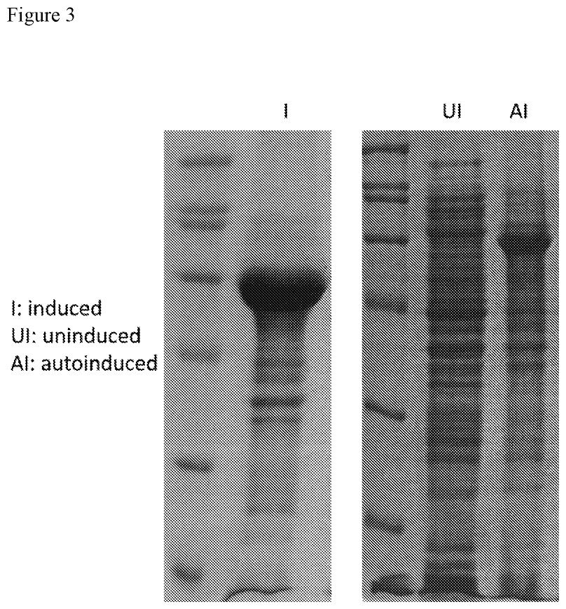

High levels of expression of the protein in E. coli were induced and this expression was confirmed, as evidenced by FIG. 3. It is expected that improvements is solubilization of the protein can be achieved using a variety of known approaches in view of the present disclosure, including but not necessarily by moving the position of the histidine tag, and/or by expression in a different vector, such as pET26-Ub-CHIS which also utilizes a ubiquitin tag and has been used successfully for the expression of poliovirus RdRp as well as other viral polymerases.

### EXAMPLE 4

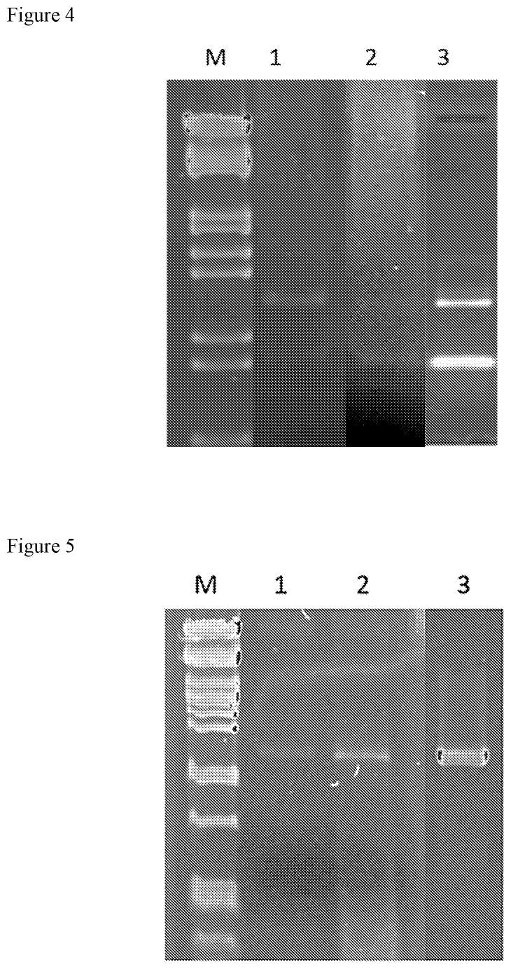

This Example is illustrated by the results presented in FIG. 4. To obtain these results, RT-PCR products were produced using Zea mays chrysovirus 1 as a template, and primers for the RdRp gene. The following were used as reverse transcriptase: M, marker lane. 1, PCV1 virions; 2, PdPV-pa virions; 3, MMuLV RT (New England Biolabs). Expected size band is ˜500 nt (upper band in gel).

### EXAMPLE 5

This Example is illustrated by the results presented in FIG. 4, lane 2. In this example we used virus particles from PdPV-pa, purified according to our published methods, in place of PCV1 virions in the above example with the Zea mays chrysovirus 1 template and primers. This reaction was carried out at room temperature, but the fungal host of PdPV-pa has a temperature optimum of 10° C. RT reactions at this temperature would like have extremely high fidelity. The amino acid sequence of PdPV-pa RdRp is provided as SEQ ID NO:2.

### EXAMPLE 6

This Example is illustrated by the results presented in FIG. 5. To obtain these results, RT-PCR product from in vitro translated PCV1 RdRp was generated. This was performed using PCV1 dsRNA as template and primers for the RdRp gene. FIG. 5 has the following features: M, marker; 1, 1 μl of in vitro translation product; 2, 2 μl of in vitro translation product; 3, MMuLV RT (New England Biolabs).

While the disclosure has been particularly shown and described with reference to specific embodiments, it should be understood by those having skill in the art that various changes in form and detail may be made therein without departing from the spirit and scope of the present disclosure as disclosed herein.

1. A purified or recombinant RNA dependent RNA polymerase (RdRp) that has reverse-transcriptase (RT) activity for use in producing cDNA from RNA, wherein the RdRp optionally comprises a purification tag.

2. The purified or recombinant RdRp of claim 1, wherein the RdRp is present in or provided with one or more buffers comprising deoxyribonucleotide triphosphates (dNTPs), and wherein the dNTPs optionally comprise all of deoxyadenosine triphosphate (dATP), deoxycytidine triphosphate (dCTP), deoxyguanosine triphosphate (dGTP), and deoxythymidine triphosphate (dTTP), and wherein the one or more buffers are free of added Uridine-5′-triphosphate (UTP).

3. The purified or recombinant RdRp of claim 1, wherein the RdRp comprises the purification tag.

4. The purified or recombinant RdRp of claim 1, wherein the RdRp is present in a buffer that comprises dATP, dCTP, dGTP, and dTTP,

5. The purified or recombinant RdRp of claim 4, wherein the RdRp is in a complex with a double-stranded RNA or single-stranded template.

6. The purified or recombinant RdRp of claim 5, wherein the complex of the RdRp and the dsRNA template further comprises a segment of a cDNA that is complementary to one strand of the dsRNA template.

7. An expression vector encoding an RdRp of claim 1.

8. A cell comprising an expression vector of claim 7.

9. An in vitro method for producing a cDNA comprising contacting an RNA template with an RdRp of claim 1 such that the cDNA is produced.

10. The method of claim 9, wherein the contacting the RNA template with the RdRp is performed in a reaction buffer comprising dATP, dCTP, dGTP, and dTTP.

11. The method of claim 10, wherein the contacting the RNA template with the RdRp is performed at a temperature of 10-25° C.

12. The method of claim 10, further comprising separating the cDNA from the reaction buffer, and optionally determining the sequence of the cDNA.

13. The method of claim 10, wherein the cDNA is generated in a one-step RT polymerase chain (PCR) reaction.

14. The method of claim 10, wherein the cDNA is generated in a two-step RT PCR reaction.

15. The method of claim 10, wherein the RNA is separated from a biological sample prior to producing the cDNA.

16. The method of any one of claims 9 15 claim 9, wherein the RNA is double stranded RNA.

17. A method comprising: a) contacting a plurality of distinct test agents divided into separate reactions chambers with an isolated or recombinant RT of claim 1, a dsRNA template, and a reverse transcriptase reaction buffer, b) allowing the test agents to be in contact with the RT, and subsequently, c) measuring of cDNA produced, wherein determining less cDNA relative to a control indicates the test agent is a candidate for use in inhibiting reverse transcriptase activity of the RT.

18. A kit comprising an RdRp of claim 1, wherein the kit further comprises one or more buffers that comprise dATP, dCTP, dGTP, and dTTP.

19. A recombinant or purified RdRp optionally comprising an expression tag, wherein the RdRp has an amino acid sequence that is at least 90% identical to a contiguous segment of the amino acid sequence of a Partitiviridae virus RdRp, wherein the contiguous segment comprises a reverse transcriptase domain.

20. The recombinant or purified RdRp of claim 19, wherein the contiguous segment spans at least 50 amino acids of the Partitiviridae virus RdRp.
